(19) United States
(12) Patent Application Publication
(10) Pub. No.: US 2020/0354741 A1
(43) Pub. Date: Nov. 12, 2020

(54) GENE-EDITING SYSTEMS FOR MODIFYING A SCN9A OR SCN10A GENE AND METHODS OF USE THEREOF

(71) Applicants: Vertex Pharmaceuticals, Boston, MA (US); CRISPR Therapeutics AG, Zug (CH)

(72) Inventors: Sheena Saayman, Boston, MA (US); Stuart Cahalan, Boston, MA (US); Angela Yen, Boston, MA (US); Sophia Espanola, Boston, MA (US); Sarah Jean Spencer, Cambridge, MA (US)

(21) Appl. No.: 16/845,280

(22) Filed: Apr. 10, 2020

Related U.S. Application Data
(60) Provisional application No. 62/833,523, filed on Apr. 12, 2019.

(51) Int. Cl. C12N 15/85; C12N 9/22

(52) U.S. Cl. CPC C12N 15/85; C12N 9/22; C12N 2800/90; C12N 2800/80; C12N 2310/20

(57) ABSTRACT

Disclosed herein are highly efficient gene-editing systems for editing a voltage-gated sodium channel gene, such as sodium voltage-gated channel alpha subunit 9 (SCN9A) or sodium voltage-gated channel alpha subunit 10 (SCN10A), either in vitro or in vivo. The gene-editing systems disclosed herein comprise RNA-guided DNA endonuclease and specific guide RNAs. Also provided herein are uses of the gene-editing systems to modify the target gene, thereby alleviating pain.

### RELATED APPLICATION

This application claims the benefit under 35 U.S.C. § 119(e) of U.S. provisional application No. 62/833,523, filed Apr. 12, 2019, the entire contents of which are incorporated herein by reference.

### BACKGROUND

Gene editing (including genomic editing) is a type of genetic engineering in which nucleotide(s)/nucleic acid(s) is/are inserted, deleted, and/or substituted in a DNA sequence, such as in the genome of a targeted cell. Recent gene editing strategies which utilize RNA-guided endonucleases, such as Cas9, enable site-specific DNA modification; however, it has been found that not all RNA-guided endonuclease, guide RNA pairs edit with high efficiency. Therefore, there still remains a critical need for identifying effective RNA-guided endonuclease, guide RNA pairs that effectively modify a gene of interest.

### SUMMARY

The present disclosure is based, at least in part, on the development of efficient gene editing systems for modifying a voltage-gated sodium channel gene, such as sodium voltage-gated channel alpha subunit 9 (SCN9A) or sodium voltage-gated channel alpha subunit 10 (SCN10A). In some embodiments, the gene editing system relies on the identification of pairs of effective RNA-guided endonuclease and guide RNAs (e.g., those disclosed herein) for effective modification of a voltage-gated sodium channel gene with low off target occurrence.

As such, in some aspects, the disclosure relates to gene-editing systems for modifying a voltage-gated sodium channel gene, such as SCN9A or SCN10A. Such a gene-editing system may comprise: (a) a first polynucleotide moiety, which comprises a first nucleotide sequence encoding a RNA-guided DNA endonuclease, or the RNA-guided DNA endonuclease; and (b) a second polynucleotide moiety, which comprises a second nucleotide sequence encoding a guide RNA (gRNA).

In some embodiments, the gene-editing system may modify a SCN9A gene and comprise: (a) a first polynucleotide moiety, which comprises a first nucleotide sequence encoding a RNA-guided DNA endonuclease, or the RNA-guided DNA endonuclease; and (b) a second polynucleotide moiety, which comprises a second nucleotide sequence encoding a guide RNA (gRNA), wherein the gRNA comprises the nucleotide sequence of any one of SEQ ID NOs: 1-20. A polynucleotide moiety as used herein can be an independent nucleic acid molecule. Alternatively, a polynucleotide moiety can be a portion of a nucleic acid molecule, which may contain one or more additional polynucleotide moieties.

A RNA-guided endonuclease of such a gene-editing system may be Staphylococcus pyogenes (SpCas9), which may be paired with a gRNA comprising the nucleotide sequence of any one of SEQ ID NOs: 1-10. Alternatively or in addition, a RNA-guided endonuclease of such a gene-editing system may be Staphylococcus aureus Cas9 (SaCas9), which may be paired with a gRNA comprising the nucleotide sequence of any one of SEQ ID NOs: 11-20.

In some embodiments, the gene-editing system may modify a SCN10A gene and comprise: (a) a first polynucleotide moiety, which comprises a first nucleotide sequence encoding a RNA-guided DNA endonuclease, or the RNA-guided DNA endonuclease; and (b) a second polynucleotide moiety, which comprises a second nucleotide sequence encoding a guide RNA (gRNA), wherein the gRNA comprises the nucleotide sequence of any one of SEQ ID NOs: 21-40. A RNA-guided endonuclease of such a gene-editing system may be SpCas9, which may be paired with a gRNA comprising the nucleotide sequence of any one of SEQ ID NOs: 21-30. Alternatively or in addition, a RNA-guided endonuclease of such a gene-editing system may be SaCas9, which may be paired with a gRNA comprising the nucleotide sequence of any one of SEQ ID NOs: 31-40.

In some embodiments, the first nucleotide sequence encoding the RNA-guided DNA endonuclease in (a) may further comprise a nucleotide sequence encoding a nuclear localization signal (NLS), which is fused in-frame with the RNA-guided DNA endonuclease. In some embodiments, the NLS is a SV40 NLS.

In some embodiments, the second nucleotide sequence in (b) may further comprise a scaffold sequence. In some examples, the scaffold sequence may be recognizable by SaCas9. Such a scaffold sequence may comprise the nucleotide sequence of GUUUUAGUACUCUGGAAACAGAAUCUACUAAAACAAGGCAAAAUGCCGUGUUUAUC UCGUCAACUUGUUGGCGAGAUUUU (SEQ ID NO: 41). In other examples, the scaffold sequence may be recognizable by SpCas9. It should be understood that because the second nucleotide sequence encoding the gRNA can be either a DNA sequence or a RNA sequence, any of the uracils (U) in this sequence may be replaced with a thymine (T).

In some embodiments, the first polynucleotide moiety of (a) and the second polynucleotide moiety of (b) are different polynucleotides, at least one of which may be a vector. A vector may be a viral vector, for example an adeno-associated viral (AAV) vector. In some embodiments, the first polynucleotide moiety of (a) and the second polynucleotide moiety of (b) are different AAV vectors.

In some embodiments, a single polynucleotide comprises the first polynucleotide moiety of (a) and the second polynucleotide moiety of (b). The single polynucleotide may be a vector, which may be a viral vector such as an AAV vector. In some embodiments, the AAV is AAV1.

Also within the scope of the present disclosure are nucleic acids and viral particles or sets of viral particles, which collectively comprise any of the gene-editing systems disclosed herein. In some embodiments, the viral particle is, or set of viral particles are, AAV particle(s).

In yet other aspects, the disclosure relates to methods of editing a voltage-gated sodium channel gene, such as SCN9A or SCN10A, the method comprises contacting a cell with: (i) any of the gene-editing systems disclosed herein; (ii) a nucleic acid comprising the gene-editing system; or (iii) a viral particle or a set of viral particles, which collectively comprise the gene-editing system.

In some embodiments, the contacting step is performed by administering the gene-editing system of (a), the nucleic acid of (b), or the viral particle(s) of (c) to a subject in need thereof. In some embodiments, the subject is a human patient having pain.

In some embodiments, the cell is an autologous cell. Alternatively a cell may be a heterologous cell. In some embodiments, the cell is a stem cell, for example an iPSC cell or mesenchymal stem cell. In some examples, the method may further comprise administering the cell with the edited gene to a subject in need thereof (e.g., a human patient having pain).

Also within the scope of the present disclosure are uses of any of the gene-editing systems described herein or components thereof for treating pain, as well as uses thereof for manufacturing a medicament for the intended medical treatment.

The details of one or more embodiments of the disclosure are set forth in the description below. Other features or advantages of the present disclosure will be apparent from the detailed description of several embodiments and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A-1D depict on target editing efficiency of 40 prioritized gRNAs in different cell models. Prioritized gRNAs included (FIG. 1A) ten gRNAs for SpCas9 targeting SCN9A, (FIG. 1B) ten gRNAs for SpCas9 targeting SCN10A, (FIG. 1C) ten gRNAs for SaCas9 targeting SCN9A, and (FIG. 1D) ten gRNAs for SaCas9 targeting SCN10A. These gRNAs were screened in iPSCs, iPSCs stably expressing Cas9, and iPSC-derived sensory neurons. Values represent mean±standard deviation.

### DETAILED DESCRIPTION

Gene editing (including genomic editing) is a type of genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell (e.g., in a targeted gene or targeted DNA sequence). When a sequence of an endogenous gene is edited, for example by deletion, insertion or substitution of nucleotide(s)/nucleic acid(s), the endogenous gene comprising the affected sequence may be knocked-out or knocked-down due to the sequence alteration. Therefore, targeted editing may be used to disrupt endogenous gene expression. Alternatively or in addition, a desired nucleic acid may be inserted into a target site in a DNA sequence (e.g., in an endogenous gene), which is known as targeted integration. “Targeted integration” refers to a process involving insertion of one or more exogenous sequences, with or without deletion of an endogenous sequence at the insertion site. Targeted integration can result from targeted gene editing when a donor template containing an exogenous sequence is present.

The present disclosure is based, at least in part, on the development of efficient gene editing systems for modifying a voltage-gated sodium channel gene, such as sodium voltage-gated channel alpha subunit 9 (SCN9A) or sodium voltage-gated channel alpha subunit 10 (SCN10A). Sodium channels are integral membrane proteins that form ion channels through a cell's membrane. Voltage-gated sodium channels are sodium channels that are “opened” (i.e., allow the flow of sodium ions through the channel) in response to a voltage change. An alpha subunit of a sodium channel forms the core of the channel and is functional on its own (i.e., in the absence of any corresponding beta subunits or other accessory proteins). The family of sodium voltage-gated channels has nine members. The alpha subunits of these channels are Naᵥ1.1, Naᵥ1.2, Naᵥ1.3, Naᵥ1.4, Naᵥ1.5, Naᵥ1.6, Naᵥ1.7, Naᵥ1.8, and Naᵥ1.9, encoded by SCN1A, SCN2A, SCN3A, SCN4A, SCN5A, SCN8A, SCN9A, SCN10A, and SCN11A, respectively.

Naᵥ1.7 (encoded by SCN9A) is expressed, for example, in the dorsal root ganglion, the trigeminal ganglion, and the sympathetic ganglion neurons. Naᵥ1.8 (encoded by SCN10A) is expressed, for example, in the dorsal root ganglion, in unmyelinated small-diameter sensory neurons called C-fibres. Both Naᵥ1.7 and Naᵥ1.8 are involved in nociception (i.e., a sensory mechanism that provides signals that lead to the sensation of pain).

Editing the SCN9A and/or SCN10A gene using any of the methods described herein may be used to treat, prevent and/or mitigate the symptoms of diseases and disorders such as, but not limited to, Congenital Pain Insensitivity, Anosmia, As If Personality, Borderline Personality Disorder, Malignant neoplasm of breast, Non-Small Cell Lung Carcinoma, Cold intolerance, Febrile Convulsions, Diabetes, Diabetes Mellitus, Dissociative disorder, Epilepsy, Erythromelalgia, Primary Erythermalgia, Facial Pain, Herpesviridae Infections, Hereditary Sensory Autonomic Neuropathy Type 5, Hyperplasia, Neuralgia, Hereditary Sensory and Autonomic Neuropathies, Degenerative polyarthritis, Pain, Pain in limb, Postoperative Pain, Parkinson Disease, Postherpetic neuralgia, Prostatic Neoplasms, Pruritus, Seizures, Somatoform Disorder, Tobacco Use Disorder, Trigeminal Neuralgia, Synovial Cyst, Chronic pain, Acute onset pain, Paramyotonia Congenita (disorder), Malaise, Sensory Discomfort, Burning Pain, Indifference to pain, Inflammatory pain, Mechanical pain, Scalp pain, Hereditary Motor and Sensory Neuropathy Type II, Common Migraine, Absence of pain sensation, Malignant neoplasm of prostate, Pain Disorder, Knee Osteoarthritis, Neuropathy, Complex Regional Pain Syndromes, Tonic-clonic seizures, Inherited neuropathies, Prostate carcinoma, Breast Carcinoma, Infantile Severe Myoclonic Epilepsy, Myxoid cyst, Channelopathies, Paroxysmal Extreme Pain Disorder, Painful Neuropathy, Compressive Neuropathies, Congenital Indifference to Pain Autosomal Recessive, Generalized Epilepsy With Febrile Seizures Plus Type 2, Generalized Epilepsy With Febrile Seizures Plus 7, Febrile Seizures Familial 3B, and Small Fiber Neuropathy (Adult-onset is referred to as small fiber neuropathy).

Mutations in the SCN9A gene are known to cause pain perception disorders, including Primary Erythermyalgia, Paroxysmal Extreme Pain Disorder, Congenital Insensitivity to Pain, and Small Fiber Neuropathy. Gain-of-function mutations in the SCN9A gene result in spontaneous pain as observed in Primary Erythermyalgia and Paroxysmal Extreme Pain Disorder. Thus, knock-out or knock-down of the SCN9A gene in patients having Primary Erythermyalgia or Paroxysmal Pain Disorder can be used to treat, prevent and/or mitigate the associated symptoms.

Primary Erythromelalgia is a rare autosomal dominant disorder characterized by episodes of burning pain in the feet and hands in response to heat and movement. Affected individuals typically develop signs and symptoms in early childhood, although in milder cases symptoms can appear later in life. Management of this condition is mainly symptomatic. Besides avoidance of pain triggers (such as heat, exercise, and alcohol), treatment options include cooling and elevating the extremity, use of anesthetics such as lidocaine and mexilitine, and use of opioid drugs in extreme cases.

Paroxysmal Extreme Pain Disorder is another rare disorder characterized by severe episodic pain in rectal, ocular, and mandibular regions as well as skin redness. Symptoms of this condition often begin in the neonatal period or in the early childhood, and can retain throughout life. Agents for treating chronic neuropathic pain disorders are often used to alleviate the pain episodes caused by the disease. Carbamazepine, a sodium channel blocker, has proven most effective of these treatments.

Mutations in the SCN10A gene are also known to cause pain perception disorders, including Familial Episodic Pain Syndrome Type 2 and Small Fiber Neuropathy. Thus, knock-out or knock-down of the SCN10A gene in patients having Familial Episodic Pain Syndrome Type 2 or Small Fiber Neuropathy can be used to treat, prevent and/or mitigate the associated symptoms.

Familial Episodic Pain Syndrome Type 2 is a rare autosomal dominant neurologic disorder characterized by adult-onset of paroxysmal pain in the feet region. The episodes are generally triggered by heat, cold, chemicals and certain surfaces. Patients may also develop hypersensitivity to touch and elevated response to pain stimulus. Currently no treatment is available for this disease. Warmth has been shown to relieve the pain episodes.

Small Fiber Neuropathy is a condition characterized by severe pain attacks and insensitivity to pain. The pain attacks are usually described as numbness, stabbing or burning, or abnormal skin sensations such as tingling or itchiness. Currently, there is no cure for small fiber peripheral neuropathy. Treatment options include intravenous immunoglobulin (IVIG) and plasmapheresis.

As described herein, indel rates and indel patterns were determined for gene editing systems comprising pairs of RNA-guided endonuclease (e.g., SpCas9 or SaCas9) and specific guide RNAs. The gene-editing systems described herein rely on the identification of specific pairs of effective RNA-guided endonuclease and guide RNAs pairs (e.g., those disclosed herein) that facilitate effective modification of a voltage-gated sodium channel gene, such as SCN9A or SCN10A, with low off target occurrence.

Accordingly, provided herein are gene-editing systems for efficient modification of voltage-gated sodium channel genes and uses thereof. Components of the gene-editing systems and genetically modified cells resulting from application of the gene-editing systems are also within the scope of the present disclosure.

### I. Gene-Editing Systems for Genetic Modification of a Voltage-Gated Sodium Channel Gene

In some aspects, the disclosure relates to gene-editing systems for modifying a voltage-gated sodium channel gene, such as sodium voltage-gated channel alpha subunit 9 (SCN9A) or sodium voltage-gated channel alpha subunit 10 (SCN10A). A “gene-editing system” refers to a combination of components for editing a target gene (e.g., SCN9A or SCN10A), or one or more agents for producing such components. For example, a gene-editing system may comprise: (a) a nuclease, or an agent for producing such (e.g., a nucleic acid encoding the nuclease); and/or (b) a guide RNA (gRNA), or an agent for producing such (e.g., a vector capable of expressing the gRNA).

The gene-editing systems as described herein may exhibit one or more advantageous in modifying a voltage-gated sodium channel gene, such as SCN9A or SCN10A. For example, it would achieve a high gene editing rate, such as frameshift-causing indel rates (e.g., at least 20%, at least 21%, at least 22%, at least 23%, at least 24%, at least 25%, at least 30%, at least 35%, or at least 40%, at least 45%, at least 50%, at least 55%, at least 60%, at least 65%, at least 70%, or at least 75% as assessed by methods described herein or known in the art) or such as total indel rates (e.g., at least 20%, at least 21%, at least 22%, at least 23%, at least 24%, at least 25%, at least 30%, at least 35%, or at least 40%, at least 45%, at least 50%, at least 55%, at least 60%, at least 65%, at least 70%, at least 75%, at least 80%, or at least 85% as assessed by methods described herein or known in the art). Further, cells edited by the gene-editing system disclosed herein may have a high survival rate (e.g., at least 50%, at least 55%, at least 60%, at least 65%, at least 70%, at least 75%, at least 80%, at least 85%, at least 85%, at least 90%, at least 95%, or at least 99%) relative to an unedited control.

In one exemplary embodiment, a gene-editing system as described herein may comprise: (a) an endonuclease (e.g., a RNA-guided DNA endonuclease) or an agent producing such (e.g., a polynucleotide coding for the endonuclease); and (b) a gRNA or an agent producing such (e.g., a vector for expressing the gRNA). Moreover, any of the gene editing systems described herein may further comprise a polynucleotide sequence encoding a donor template. In some examples, the gene-editing system described herein comprises an endonuclease, a gRNA, and optionally a donor template. Such a gene-editing system may comprise one polynucleotide that provides the donor template and produces the gRNA. Alternatively, the gene-editing system may comprise the donor template and a separate nucleic acid, which can be the gRNA per se, or a polynucleotide that produces the gRNA. In other examples, the gene-editing system may comprise one or more polynucleotides, which collectively produces the endonuclease, the gRNA, and optionally the donor template. In some examples, the gene-editing system may comprise a polynucleotide comprising a first polynucleotide sequence encoding an endonuclease and a second polynucleotide sequence encoding a gRNA. Alternatively, the gene-editing system may comprise two polynucleotides: the first comprising a first polynucleotide sequence encoding an endonuclease and the second comprising a second polynucleotide sequence encoding a gRNA.

A. RNA-Guided Endonucleases

RNA-guided endonucleases are enzymes that utilize RNA:DNA base-pairing to target and cleave a polynucleotide. RNA-guided endonuclease may cleave single-stranded polynucleic acids or at least one strand of a double-stranded polynucleotide. A gene editing-system may comprise one RNA-guided endonuclease. Alternatively, a gene-editing system may comprise at least two (e.g., two, three, four, five, six, seven, eight, nine, ten, or more than ten) RNA-guided endonucleases.

The CRISPR-Cas9 system is a naturally-occurring defense mechanism in prokaryotes that has been repurposed as a RNA-guided DNA-targeting platform used for gene editing. It relies on the DNA nuclease Cas9, and two noncoding RNAs—crisprRNA (crRNA) and trans-activating RNA (tracrRNA)—to target the cleavage of DNA. crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA if the target sequence is followed by a specific short DNA motif (with the sequence NGG) referred to as a protospacer adjacent motif (PAM). TracrRNA hybridizes with the 3′ end of crRNA to form a RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

A gene-editing system may comprise a CRISPR endonuclease (e.g., a CRISPR associated protein 9 or Cas9 nuclease). In some embodiments, the endonuclease is from Streptococcus aureus (e.g., saCas9) or Streptococcus pyogenes (e.g., spCas9), although other CRISPR homologs may be used. It should be understood that a Cas9 may be substituted with another RNA-guided endonuclease known in the art, such as Cpf1. Finally, it should be understood, that a wild-type RNA-guided endonuclease may be used or modified versions may be used (e.g., evolved versions of Cas9, Cas9 orthologues, Cas9 chimeric/fusion proteins, or other Cas9 functional variants). For example, in some embodiments, the RNA-guided endonuclease is modified to comprise a nuclear localization signal (NLS), such as an SV40 NLS or a NucleoPlasmine NLS. Examples of other nuclear localization signals are known to those having skill in the art. In some embodiments, the NLS comprises an SV40 NLS and a NucleoPlasmine NLS.

B. Guide RNA

The present disclosure provides a genome-targeting nucleic acid, or an agent for producing such (e.g., a polynucleotide comprising a nucleotide sequence encoding a gRNA), that can direct the activities of an associated polypeptide (e.g., a RNA-guided endonuclease) to a specific target sequence within a target nucleic acid. The genome-targeting nucleic acid can be a RNA. A genome-targeting RNA is referred to as a “guide RNA” or “gRNA” herein. In some embodiments, a gene-editing system comprises one gRNA. In other embodiments, a gene-editing system comprises at least two gRNAs (e.g., two, three, four, five, six, seven, eight, nine, ten, or more than ten gRNAs).

A gRNA of a gene-editing system may be provided in a synthesized form. For example, a guide RNA may be synthesized by chemical means, as illustrated below and described in the art. While chemical synthetic procedures are continually expanding, purifications of such RNAs by procedures such as high performance liquid chromatography (which avoids the use of gels such as PAGE) tends to become more challenging as polynucleotide lengths increase significantly beyond a hundred or so nucleotides. One approach used for generating RNAs of greater length is to produce two or more molecules that are ligated together. Much longer RNAs are more readily generated enzymatically. Various types of RNA modifications can be introduced during or after chemical synthesis and/or enzymatic generation of RNAs, e.g., modifications that enhance stability, reduce the likelihood or degree of innate immune response, and/or enhance other attributes, as described in the art.

Alternatively, a gene-editing system may comprise an agent for the production of a gRNA. For example, a gene-editing system may comprise a nucleotide sequence encoding the nucleotide sequence of a gRNA and an additional nucleotide sequence that facilitates expression/production of the gRNA.

A gRNA may be a double-molecule guide RNA. A double-molecule gRNA comprises two strands of RNA. The first strand may comprise in the 5′ to 3′ direction, an optional spacer extension sequence, a spacer sequence and a scaffold sequence comprising a minimum CRISPR repeat sequence. The second strand comprises a minimum tracrRNA sequence (complementary to the minimum CRISPR repeat sequence), a 3′ tracrRNA sequence, and an optional tracrRNA extension sequence.

Alternatively, a gRNA may be a single-molecule guide RNA (sgRNA) comprising a spacer sequence and a scaffold sequence. The scaffold sequence may comprise a tracrRNA sequence as described herein. A sgRNA (e.g., in a Type II system) may comprise, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. Alternatively, a sgRNA (e.g., in a Type V system) may comprises, in the 5′ to 3′ direction, a minimum CRISPR repeat sequence and a spacer sequence.

The single-molecule gRNA can comprise no uracil at the 3′ end of the gRNA sequence. Alternatively, the gRNA can comprise one or more uracil at the 3′ end of the gRNA sequence. For example, the gRNA can comprise 1 uracil (U) at the 3′ end of the gRNA sequence. The gRNA can comprise 2 uracil (UU) at the 3′ end of the gRNA sequence. The gRNA can comprise 3 uracil (UUU) at the 3′ end of the gRNA sequence. The gRNA can comprise 4 uracil (UUUU) at the 3′ end of the gRNA sequence. The gRNA can comprise 5 uracil (UUUUU) at the 3′ end of the gRNA sequence. The gRNA can comprise 6 uracil (UUUUUU) at the 3′ end of the gRNA sequence. The gRNA can comprise 7 uracil (UUUUUUU) at the 3′ end of the gRNA sequence. The gRNA can comprise 8 uracil (UUUUUUUU) at the 3′ end of the gRNA sequence.

It is further understood that the nucleotides of the gRNAs described above may comprise modified nucleic acids at any nucleotide position. Accordingly, a gRNA can be unmodified or modified. For example, modified gRNAs can comprise one or more 2′-O-methyl phosphorothioate nucleotides. Examples of additional modified nucleic acids are known to those having skill in the art. See, e.g., WO2018007976 and WO2018007980, the relevant disclosures of each of which are incorporated by reference for the purpose and/or subject matter referenced herein.

(i) gRNA Spacer

As is understood by the person of ordinary skill in the art, each gRNA is designed to include a spacer sequence complementary to its genomic target sequence. See Jinek et al., Science, 337, 816-821 (2012) and Deltcheva et al., Nature, 471, 602-607 (2011). A spacer sequence is a nucleotide sequence that defines the target sequence (e.g., a DNA target sequences, such as a genomic target sequence) of a target nucleic acid of interest. The gRNA can comprise a variable length spacer sequence with 17-30 nucleotides at the 5′ end of the gRNA sequence. In some embodiments, the spacer sequence is 15 to 30 nucleotides. In some embodiments, the spacer sequence is 15, 16, 17, 18, 19, 29, 21, 22, 23, 24, 25, 26, 27, 28, 29, or 30 nucleotides. In some embodiments, a spacer sequence is 20 nucleotides.

The “target sequence” is adjacent to a PAM sequence and is the sequence modified by a RNA-guided nuclease (e.g., Cas9). The “target nucleic acid” is a double-stranded molecule: one strand comprises the target sequence and is referred to as the “PAM strand,” and the other complementary strand is referred to as the “non-PAM strand.” One of skill in the art recognizes that the gRNA spacer sequence hybridizes to the reverse complement of the target sequence, which is located in the non-PAM strand of the target nucleic acid of interest. Thus, the gRNA spacer sequence is the RNA equivalent of the target sequence. For example, if the target sequence is 5′-AGAGCAACAGTGCTGTGGCC-3′ (SEQ ID NO: 498), then the gRNA spacer sequence is 5′-AGAGCAACAGUGCUGUGGCC-3′ (SEQ ID NO: 499). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

The spacer sequence is designed to hybridize to a region of the target nucleic acid that is located 5′ of a PAM of the Cas9 enzyme used in the system. The spacer may perfectly match the target sequence or may have mismatches. Each Cas9 enzyme has a particular PAM sequence that it recognizes in a target DNA. For example, S. pyogenes Cas9 recognizes in a target nucleic acid a PAM that comprises the sequence 5′-NRG-3′, where R comprises either A or G, where N is any nucleotide and N is immediately 3′ of the target nucleic acid sequence targeted by the spacer sequence. The canonical PAM for S. pyogenes Cas9 is 5′-NGG-3′, but as indicated in the preceding sentence, S. pyogenes Cas9 can also recognize the non-canonical PAM 5′-NAG-3′. Similarly, for S. aureus Cas9 the PAM comprises the sequence 5′-NNGRRT-3′.

In some embodiments, the target nucleic acid sequence comprises 20-22 nucleotides. In some embodiments, the target nucleic acid comprises less than 20 nucleotides. In some embodiments, the target nucleic acid comprises more than 20 nucleotides. In some embodiments, the target nucleic acid comprises at least: 5, 10, 15, 16, 17, 18, 19, 20, 21, 22, 23, 24, 25, 30 or more nucleotides. In some embodiments, the target nucleic acid comprises at most: 5, 10, 15, 16, 17, 18, 19, 20, 21, 22, 23, 24, 25, 30 or more nucleotides. In some embodiments, the target nucleic acid sequence comprises 20-22 bases immediately 5′ of the first nucleotide of the PAM. For example, in a sequence comprising 5′-NNNNNNNNNNNNNNNNNNNNNRG-3′ (SEQ ID NO: 489) or 5′-NNNNNNNNNNNNNNNNNNNNNNNNGRRT-3′ (SEQ ID NO: 490), the target nucleic acid comprises the sequence that corresponds to the Ns lacking an underscore, wherein N is any nucleotide, and the underlined NRG sequence and NNGRRT sequence is the S. pyogenes PAM and the S. aureus PAM, respectively.

In some embodiments, a gRNA used herein may comprise a spacer sequence of 20 nucleotides. In some embodiments, such a gRNA is used with a SpCas9. In other embodiments, a gRNA used herein may comprise a spacer sequence of 22 nucleotides. In some embodiments, such a gRNA is used with a SaCas9.

In some embodiments, a gRNA used herein may comprise a spacer sequence listed in Tables 1-4. In some examples, a gRNA used herein may comprise a spacer sequence listed in Table 1 in combination with SpCas9 for editing SCN9A. In some examples, a gRNA used herein may comprise a spacer sequence listed in Table 2 in combination with SaCas9 for editing SCN9A. In some examples, a gRNA used herein may comprise a spacer sequence listed in Table 3 in combination with SpCas9 for editing SCN10A. In some examples, a gRNA used herein may comprise a spacer sequence listed in Table 4 in combination with SaCas9 for editing SCN10A. Any of these gRNAs may comprise a spacer sequence listed in any of Tables 1 and 3 (in combination with SpCas9 enzyme) with greater than 40% (e.g., 50%, 55%, 60%, 65%, 70%, 75%, 80%, or greater) mean total Indel percentage and/or with greater than 40% (e.g., 50%, 55%, 60%, 65%, 70%, 75%, or greater) mean frameshift-causing Indel percentage. Alternatively, any of these gRNAs may comprise a spacer sequence listed in any of Tables 2 and 4 (in combination with SaCas9 enzyme) with greater than 15% (e.g., 20%, 25%, 30%, 35%, 40%, 45%, 50%, 55%, or greater) mean total Indel percentage and/or with greater than 15% (e.g., 20%, 25%, 30%, 35%, 40%, 45%, or greater) mean frameshift-causing Indel percentage.

Exemplary gRNAs may comprise one of the following spacer sequences:

(SEQ ID NO: 1)
CAAUUUGGGUGGUACCUGAU;
(SEQ ID NO: 2)
GCUUCGCCUUGCAGAAAACA;
(SEQ ID NO: 3)
GCCUAUGCCCUUCGACACCA;
(SEQ ID NO: 4)
AUAGGCGAGCACAUGAAAAG;
(SEQ ID NO: 5)
CGGCUGAAUAUACAAGUAUU;
(SEQ ID NO: 6)
GGAACACCACCCAAUGACUG;
(SEQ ID NO: 7)
CAGGCCUGAAGACAAUUGUA;
(SEQ ID NO: 8)
GGAAUGUCCCCAUAGAUGAA;
(SEQ ID NO: 9)
CCACCAAUGCUGCCGGUGAA;
(SEQ ID NO: 10)
CAGUCACCACUCAGCAUUCG;
(SEQ ID NO: 11)
AAGCAGAAUUAUGGGCCUCUCA;
(SEQ ID NO: 12)
GCCUUGCAGAAAACAAGGAGCC;
(SEQ ID NO: 13)
ACGACAAAAUCCAGCCAGUUCC;
(SEQ ID NO: 14)
CUGGGAAAACCUUUACCAACAG;
(SEQ ID NO: 15)
UCCCAACCUCAGACAGAGAGCA;
(SEQ ID NO: 16)
GAUGUUACUGCUGCGUCGCUCC;
(SEQ ID NO: 17)
CAUGAUCCUGACUGUGUUCUGU;
(SEQ ID NO: 18)
CUCGUGUGUAGUCAGUGUCCAG;
(SEQ ID NO: 19)
AAACUGAUUGCCAUGGAUCCAU;
(SEQ ID NO: 20)
AGAAAACAAGGAGCCACGAAUG;
(SEQ ID NO: 21)
GCUCCCCGAUCAGUUCUGCU;
(SEQ ID NO: 22)
UGUAGUCACCAUGGCGUAUG;
(SEQ ID NO: 23)
GGAAGCUCCGCAGCACAGAC;
(SEQ ID NO: 24)
UCCUUACAACCAGCGCAGGA;
(SEQ ID NO: 25)
ACUUCUGACCCCUUACUGUG;
(SEQ ID NO: 26)
GAGCUCCCAGCAGAACUGAU;
(SEQ ID NO: 27)
CCGAGACAUCGACAGCUCCA;
(SEQ ID NO: 28)
AUCCGUUCUACAGCACACAC;
(SEQ ID NO: 29)
UCACGUACCUGAGAGAUCCU;
(SEQ ID NO: 30)
CGCAGGUGCUAGCAGCACUA;
(SEQ ID NO: 31)
CCCUGGAGCUGUCGAUGUCUCG;
(SEQ ID NO: 32)
UAGAUCCGUUCUACAGCACACA;
(SEQ ID NO: 33)
AGUGAGAGGAAAGCCCAAGCAA;
(SEQ ID NO: 34)
ACCUUUCCGGGCCCAAAGGGCA;
(SEQ ID NO: 35)
CUUUGACUGCAUCAUCGUCACU;
(SEQ ID NO: 36)
CACUUCUUCUGGAAAUAAUAGU;
(SEQ ID NO: 37)
AUUUUAGCGUCAUUACCCUGGC;
(SEQ ID NO: 38)
AACAACUUCCGUCGCUUUACUC;
(SEQ ID NO: 39)
GCCGAGAUAUCUCACUCCCUGA;
(SEQ ID NO: 40)
UGGUGUUCAUCUUCUCCAUGCC.

(ii) gRNA Scaffold

In some embodiments, the gRNA further comprises a scaffold sequence. A scaffold sequence may comprise the sequence of a minimum CRISPR repeat sequence, a single-molecule guide linker, a minimum tracrRNA sequence, a 3′ tracrRNA sequence, and/or an optional tracrRNA extension sequence. Exemplary scaffold sequences for various CRISPR proteins are known to those of ordinary skill in the art.

Selection of a scaffold sequence may depend on the RNA-guided DNA endonuclease to be used in the gene editing system as used herein, e.g., SaCas9 or SpCas9, which is known to those skilled in the art. For example, if SpCas9 is to be used, a scaffold sequence recognizable by SpCas9 can be selected. Examples of SpCas9 scaffold sequences are known in the art. See, e.g., Zhang et al., Plant Mol Biol. 2018; 96(4): 445-456; www.addgene.org. One exemplary scaffold sequence in a single-molecule guide RNA may comprise the nucleotide sequence of

(SEQ ID NO: 42)
GTTTAAGAGCTATGCTGGAAACAGCATAGCAAGTTTAAATAAGGCTAGT
CCGTTATCAACTTGAAAAAGTGGCACCGAGTCGGTGC;

Alternatively, if a SaCas9 endonuclease is to be used, a scaffold sequence recognizable by the SaCas9 can be selected. A scaffold sequence in a single-molecule guide RNA for SaCas9 may comprise the nucleic acid sequence of GUUUUAGUACUCUGGAAACAGAAUCUACUAAA ACAAGGCAAAAUGCCGUGUUUAUCUCGUCAACUUGUUGGCGAGAUUUU (SEQ ID NO: 41). A single-molecule guide RNA may further comprise an optional spacer extension.

It should be understood that because the nucleotide sequence encoding a gRNA can be either a DNA sequence or a RNA sequence, any of the uracils (U) in the sequences describing a gRNA may be replaced with a thymine (T). Likewise, any T (thymine) in a sequence referring to gRNAs would refer to U (or uracil) in the context of RNA molecules. Sequences containing T (thymine) herein would encompass both DNA molecules and RNA molecules (wherein T refers to U).

(iii) Exemplary RNA-Guided Endonuclease-gRNA Pairs

In some embodiments, the gene editing system relies on the identification of effective RNA-guided endonuclease, guide RNAs pairs (e.g., those disclosed herein) for effective modification of a voltage-gated sodium channel gene.

For example, a gene editing system for modifying a sodium voltage-gated channel alpha subunit 9 (SCN9A) gene may comprise a Staphylococcus pyogenes (SpCas9) and a gRNA comprising the nucleotide sequence of any one of SEQ ID NOs: 1-10. Alternatively or in addition, a gene editing system for modifying a sodium voltage-gated channel alpha subunit 9 (SCN9A) gene may comprises a Staphylococcus aureus (SaCas9) and a gRNA comprising the nucleotide sequence of any one of SEQ ID NOs: 11-20.

In another example, a gene editing system for modifying a sodium voltage-gated channel alpha subunit 10 (SCN10A) gene may comprise a SpCas9 and a gRNA comprising the nucleotide sequence of any one of SEQ ID NOs: 21-30. Alternatively or in addition, a gene editing system for modifying a sodium voltage-gated channel alpha subunit 10 (SCN10A) gene may comprise a SaCas9 and a gRNA comprising the nucleotide sequence of any one of SEQ ID NOs: 31-40.

(iv) Ribonucleoprotein Complexes

In some instances, the gene-editing system disclosed herein may comprise a ribonucleoprotein complex (RNP), in which a gRNA and a nuclease (e.g., as described above) form a complex. As used herein, the term “ribonucleoprotein” or “RNP” refers to a protein that is structurally associated with a nucleic acid (either DNA or RNA). For example, in some embodiments, a Cas9 RNA-guided endonuclease and a gRNA of a gene-editing system are in the form of an RNP.

C. Donor Template

A donor template comprises a nucleic acid sequence that is to be inserted into a target site in a DNA sequence (e.g., in an endogenous gene). A donor template of a gene-editing system may be provided in a synthesized form. Alternatively, a gene-editing system may comprise an agent (e.g., a nucleic acid such as a vector) for the production of a donor template. For example, a gene-editing system may comprise a nucleic acid (e.g., a vector) for producing the donor template.

A donor template may comprise one or more homologous arms to allow for efficient homology dependent recombination (HDR) at a genomic location of interest. The length of a homologous arm may vary. For example, a homologous arm may be at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, or at least 1000 nucleotides in length. Likewise, a homologous arm may be 50 to 100, 50 to 200, 50 to 300, 50 to 400, 50 to 500, 50 to 600, 50 to 700, 50 to 800, 50 to 900, 50 to 1000, 100 to 200, 100 to 300, 100 to 400, 100 to 500, 100 to 600, 100 to 700, 100 to 800, 100 to 900, 100 to 1000, 200 to 300, 200 to 400, 200 to 500, 200 to 600, 200 to 700, 200 to 800, 200 to 900, 200 to 1000, 300 to 400, 300 to 500, 300 to 600, 300 to 700, 300 to 800, 300 to 900, 300 to 1000, 400 to 500, 400 to 600, 400 to 700, 400 to 800, 400 to 900, 400 to 1000, 500 to 600, 500 to 700, 500 to 800, 500 to 900, 500 to 1000, 600 to 700, 600 to 800, 600 to 900, 600 to 1000, 700 to 800, 700 to 900, 700 to 1000, 800 to 900, 800 to 1000, or 900 to 1000 nucleotides in length. In particular, a homologous arm may be 500 nucleotides in length.

For example, in some embodiments a donor template comprises a 5′ homologous arm (i.e., positioned upstream to the first nucleotide sequence) and a 3′ homologous arm (i.e., positioned downstream to the first nucleotide sequence), wherein the 5′ homologous arm comprises a nucleic acid sequence that is homologous to a region upstream to the genomic location of interest, and wherein the 3′ homologous arm comprises a nucleic acid sequence that is homologous to a region downstream to the genomic location of interest.

In other embodiments, the donor template may comprise a 5′ homologous arm and lack a 3′ homologous arm. In yet other embodiments, the donor template may comprise a 3′ homologous arm and lack a 5′ homologous arm.

Alternatively, a donor template may lack homologous arms. For example, in some instances, a donor template may be integrated by NHEJ-dependent end joining following cleavage at the target site.

A donor template may also comprise a polynucleotide sequence encoding a gene of interest, or a portion thereof (e.g., SCN9A, SCN10A, or a portion thereof). Alternatively or in addition, a donor template may comprise a polynucleotide sequence encoding a regulatory element (e.g., a regulatory element of SCN9A or SCN10A)

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, is inserted so that its expression is driven by the endogenous promoter, such as the promoter that drives expression of the endogenous gene into which the donor is inserted.

Furthermore, exogenous sequences may also include transcriptional or translational regulatory sequences, for example, promoters, enhancers, insulators, internal ribosome entry sites, sequences encoding 2A peptides and/or polyadenylation signals.

It is understood that the nucleotides of the donor templates described above may comprise modified nucleic acids at any nucleotide position.

D. Viral Vector/Viral Particle-Based Gene-Editing System

In some embodiments, the gene-editing system disclosed herein may comprise polynucleic acids (e.g., vectors such as viral vectors) or viral particles comprising such. The polynucleic acid(s) produces the components (e.g., a nuclease and a gRNA) for editing a voltage-gated sodium channel gene as described herein.

In some examples, the gene-editing system comprises one polynucleic acid capable of producing all components of the gene-editing system, including a nuclease and a gRNA. In other examples, the gene-editing system comprises two polynucleic acids, one encoding the nuclease and the other encoding the gRNA.

The nucleic acid (or at least one nucleic acid in the set of nucleic acids) may be a vector such as a viral vector, such as a retroviral vector, an adenovirus vector, an adeno-associated viral (AAV) vector, and a herpes simplex virus (HSV) vector.

In some examples, the gene-editing system may comprise one or more viral particles that carry genetic materials for producing the components of the gene-editing system as disclosed herein. A viral particle (e.g., AAV particle) may comprise one or more components (or agents for producing one or more components) of a gene-editing system (e.g., as described herein). A viral particle (or virion) comprises a nucleic acid, which encodes the viral genome, and an outer shell of protein (i.e., a capsid). In some instances, a viral particle further comprises an envelope of lipids that surround the protein shell.

In some examples, a viral particle comprises a polynucleic acid capable of producing all components of the gene-editing system, including a nuclease and a gRNA. In other examples, a viral particle comprises a polynucleic acid capable of producing one or more components of the gene-editing system. For example a viral particle may comprise a polynucleic acid capable of producing the nuclease. Alternatively, a viral particle may comprise a polynucleic acid capable of producing the gRNA.

The viral particles described herein may be derived from any viral particle known in the art including, but not limited to, a retroviral particle, an adenovirus particle, an adeno-associated viral (AAV) particle, or a herpes simplex virus (HSV) particle. In some embodiments, the viral particle is an AAV particle. In some embodiments, the AAV particle is an AAV1 particle.

In some embodiments, a set of viral particles comprises more than one gene-editing system. In some embodiments, each viral particle in the set of viral particles is an AAV particle. In other embodiments, a set of viral particles comprises more than one type of viral particle (e.g., a retroviral particle, an adenovirus particle, an adeno-associated viral (AAV) particle, or a herpes simplex virus (HSV) particle).

E. Additional Exemplary Gene-Editing Systems

In addition, the gene-editing system disclosed herein may comprise a nuclease (e.g., a Cas9 enzyme) as disclosed herein. Such a gene-editing system may further comprise the gRNA. The nuclease and the gRNA may form an RNP for delivery. Further, the gene-editing system may further comprise the gRNA and a polynucleic acid (e.g., a vector as those described herein) for producing the donor template. The nuclease and the gRNA may form an RNP complex. Alternatively, the gene-editing system may further comprise one or more polynucleic acids for producing the gRNA and the donor template.

Alternatively, the gene-editing system disclosed herein may comprise an agent for produce the nuclease, for example, an expression vector such as a viral vector as disclosed herein capable of expressing the nuclease. Such a gene-editing system may further comprise the gRNA or agents for producing such.

Any other format of the gene-editing system comprising the components as disclosed herein for modifying a voltage-gated sodium channel gene or agents producing such are within the scope of the present disclosure.

### II. Methods of Editing a Voltage-Gated Sodium Channel Gene

In some aspects, the disclosure relates to methods of editing a voltage-gated sodium channel gene, such as sodium voltage-gated channel alpha subunit 9 (SCN9A) or sodium voltage-gated channel alpha subunit 10 (SCN10A), using any of the gene-editing systems disclosed herein. An editing event may introduce a mutation or correct a mutation in a sodium voltage-gated channel (e.g., SCN9A or SCN10A). One or more copies (i.e., alleles) of a gene (e.g., SCN9A or SCN10A) may be corrected and/or mutated.

A method of editing a voltage-gated sodium channel gene may comprise contacting a cell with: a gene-editing system as described herein; a viral particle or set of viral particles comprising a gene-editing system as described herein; and/or a nucleic acid or set of nucleic acids comprising a gene-editing system as described herein. These methods may be performed, for example, on one or more cells existing within a living subject (e.g., in vivo). Alternatively or in addition, these methods may be performed on one or more cells existing in culture (e.g., ex vivo). In some instances, a cell edited in culture is then administered to a subject (categorized herein as “cell-based therapy”).

A. Delivery Methods

The contacting of the cell (or subject) with the gene-editing system, viral particle or set of viral particles, and/or nucleic acid or set of nucleic acids may be performed via various delivery methods. For example, nucleases and/or gRNAs may be delivered using a vector system, including, but not limited to, plasmid vectors, DNA minicircles, retroviral vectors, lentiviral vectors, adenovirus vectors, poxvirus vectors; herpesvirus vectors and adeno-associated virus vectors, and combinations thereof.

Conventional viral and non-viral based gene transfer methods can be used to introduce nucleic acids encoding nucleases and gRNAs in cells. Non-viral vector delivery systems include DNA plasmids, DNA minicircles, naked nucleic acid, and nucleic acid complexed with a delivery vehicle such as a liposome or poloxamer. Viral vector delivery systems include DNA and RNA viruses, which have either episomal or integrated genomes after delivery to the cell.

Methods of non-viral delivery of nucleic acids include, but are not limited to, electroporation, lipofection, microinjection, biolistics, virosomes, liposomes, immunoliposomes, polycation or lipid:nucleic acid conjugates, naked DNA, naked RNA, capped RNA, artificial virions, and agent-enhanced uptake of DNA. Sonoporation using, e.g., the Sonitron 2000 system (Rich-Mar) can also be used for delivery of nucleic acids.

Methods for delivery of proteins (e.g., RNA-guided endonucleases) include, but are not limited to, the use of cell-penetrating peptides and nanovehicles.

(i) Adeno-Associated Viral Delivery

One or more components of a gene editing system may be delivered to a cell using an adeno-associated virus (AAV). AAVs are small viruses which integrate site-specifically into the host genome and can therefore deliver a transgene. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids confer AAV serotype, which determines which target organs the capsids will primarily bind and thus what cells the AAV will most efficiently infect. There are twelve currently known human AAV serotypes. In some embodiments, the AAV is AAV serotype 6 (AAV6). In some embodiments, the AAV is AAV serotype 1 (AAV1).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

(ii) Homology-Directed Repair (HDR)

One or more components of a gene editing system may be inserted into the target genomic region of the edited cell by homology directed repair (HDR). Both strands of the DNA at the target genomic region are cut by a CRISPR Cas9 enzyme. HDR then occurs to repair the double-strand break (DSB) and insert the donor DNA. For this to occur correctly, the donor sequence is designed with flanking residues which are complementary to the sequence surrounding the DSB site in the target gene (hereinafter “homology arms”). These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

(iii) Non-Homologous End Joining (NHEJ)

The NHEJ pathway may also produce, at very low frequency, inserts containing exons 11-27. Such repair should correct expression when the insert is in the sense strand orientation.

### III. Therapeutic Applications

The gene-editing methods disclosed herein may be applied for treating a patient with pain. In some embodiments, provided herein are ex vivo cell-based therapy. In other embodiments, provided herein are in vivo gene therapy.

(i) Cells-Based Therapy

Genetically-edited cells may be produced using any of the methods described herein. In some embodiments, one or more gene edits within a population of edited cells results in a phenotype associated with changes in voltage-gated sodium channel functionality.

In some embodiments, genetically-edited cells of the present disclosure exhibit decreased voltage-gated sodium channel activity (e.g., decreased by at least 5%, 10%, 20%, 30%, 40%, 50%, 60%, 70%, 80%, 90%, 95%, or 100%) relative to the unedited control. For example, the levels of Naᵥ1.7 and/or Naᵥ1.8 activity may be decreased by at least 5%, 10%, 20%, 30%, 40%, 50%, 60%, 70%, 80%, 90%, 95%, or 100% relative to control unedited cells. In some embodiments, the levels of Naᵥ1.7 and/or Naᵥ1.8 activity may be decreased by 5%-10%, 5%-20%, 5%-30%, 5%-40%, 5%-50%, 5%-60%, 5%-70%, 5%-80%, 5%-90%, 10%-20%, 10%-30%, 10%-40%, 10%-50%, 10%-60%, 10%-70%, 10%-80%, 10%-90%-20%-30%, 20%-40%, 20%-50%, 20%-60%, 20%-70%, 20%-80%, 20%-90%, 30%-40%, 30%-50%, 30%-60%, 30%-70%, 30%-80%, 30%-90%, 40%-50%, 40%-60%, 40%-70%, 40%-80%, 40%-90%, 50%-50%, 50%-70%, 50%-80%, or 50%-90%, relative to control T cells.

In other embodiments, genetically-edited cells of the present disclosure exhibit increased voltage-gated sodium channel activity (e.g., by at least 30%, 50%, 100%, 2-fold, 5-fold, or 10-fold) relative to the unedited control. For example, the levels of Naᵥ1.7 and/or Naᵥ1.8 activity may be increased by at least 30%, at least 50%, at least 100%, at least 200%, at least 500%, at least 1000% relative to control unedited cells. In some embodiments, the levels of Naᵥ1.7 and/or Naᵥ1.8 activity may be increased by 30%-50%, 30%-100%, 30%-200%, 30%-500%, 30%-1000%, 50%-100%, 50%-200%, 50%-500%, 50%-1000%, 100%-200%, 100%-500%, 100%-1000%, 200%-500%, 200%-1000%, or 500%-1000% relative to control unedited cells.

In some embodiments, a biopsy of the patient's peripheral nerves can be performed. The nerve tissue can be isolated from the patient's skin or leg. Then, a cell of the peripheral nervous system (e.g., a neuron or a glial cell such as Schwann cell in nerves or satellite glial cell in ganglia) is isolated from the biopsied material. Then, the chromosomal DNA of the cell of the peripheral nervous system (e.g., a neuron, or a glial cell such as Schwann cell in nerves or satellite glial cell in ganglia) can be edited using the materials and methods described herein. Finally, the edited cell of the peripheral nervous system (e.g., a neuron or a glial cell such as Schwann cell in nerves or satellite glial cell in ganglia) is implanted into the patient. Any source or type of cell may be used as the progenitor cell.

In other embodiments, a patient specific induced pluripotent stem cell (iPSC) can be created. Then, the chromosomal DNA of these iPSC cells can be edited using the materials and methods described herein. Next, the genome-edited iPSCs can be differentiated into cells of the peripheral nervous system (e.g., a neuron or a glial cell such as Schwann cell in nerves or satellite glial cell in ganglia). Finally, the differentiated cells of the peripheral nervous system (e.g., a neuron or a glial cell such as Schwann cell in nerves or satellite glial cell in ganglia) are implanted into the patient.

Alternatively, a mesenchymal stem cell can be isolated from the patient, which can be isolated from the patient's bone marrow or peripheral blood. Next, the chromosomal DNA of these mesenchymal stem cells can be edited using the materials and methods described herein. Next, the genome-edited mesenchymal stem cells can be differentiated into cells of the peripheral nervous system (e.g., a neuron or a glial cell such as Schwann cell in nerves or satellite glial cell in ganglia). Finally, the differentiated cells of the peripheral nervous system (e.g., a neuron or a glial cell such as Schwann cell in nerves or satellite glial cell in ganglia) are implanted into the patient.

Any of the genetically edited cells may be administered to a subject. The step of administering may include the placement (e.g., transplantation) of genetically engineered cells into a subject, by a method or route that results in at least partial localization of the introduced cells at a desired site, such that a desired effect(s) is produced and where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. In some embodiments, the administration is to the respiratory tract of the subject.

Modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

In some embodiments, genetically engineered cells are administered systemically, which refers to the administration of a population of cells other than directly into a target site, tissue, or organ, such that it enters, instead, the subject's circulatory system and, thus, is subject to metabolism and other like processes.

For use in the various aspects described herein, an effective amount of genetically engineered cells comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. In some examples described herein, the cells are expanded in culture prior to administration to a subject in need thereof.

(ii) In Vivo Gene Therapy

Alternatively, the gene-editing methods and materials disclosed herein can be applied to genetically modifying the target gene (SCN9A or SCN10A) in vivo. Chromosomal DNA of the cells in a patient can be edited using the materials and methods described herein. In some aspects, the target cell in an in vivo based therapy can be a neuron of the peripheral nervous system.

Although certain cells present an attractive target for ex vivo treatment and therapy, increased efficacy in delivery may permit direct in vivo delivery to such cells. Ideally the targeting and editing would be directed to the relevant cells. Cleavage in other cells can also be prevented by targeted delivery and/or the use of promoters only active in certain cells and or developmental stages. Additional promoters are inducible, and therefore can be temporally controlled if the nuclease is delivered as a plasmid. The amount of time that delivered RNA and protein remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment and post-engraftment integration of neurons and glial cells appropriately into existing brain circuits.

In some aspects, the disclosure relates to methods of administering an effective amount of a gene-editing system as descried herein, a viral particle or set of viral particles comprising a gene-editing system as described herein, a nucleic acid or set of nucleic acids comprising a gene-editing system as described herein, or a composition of edited cells as described herein to a subject in need thereof.

A subject may be any subject for whom diagnosis, treatment, or therapy is desired. In some embodiments, the subject is a mammal. In some embodiments, the subject is a human. In some embodiments, the subject is a human patient having pain. In some embodiments, the human patient is a child.

An effective amount refers to the amount of a gene-editing system, a viral particle or set of viral particles comprising a gene-editing system, a nucleic acid or set of nucleic acids comprising a gene-editing system, or a population of genetically engineered cells needed to prevent or alleviate at least one or more signs or symptoms of a medical condition (i.e., pain), and relates to a sufficient amount of a composition to provide the desired effect (i.e., to treat a subject having pain). An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

The efficacy of a treatment comprising a composition for the treatment of a medical condition can be determined by the skilled clinician. A treatment is considered an “effective treatment,” if any one or all of the signs or symptoms of, as but one example, levels of functional target are altered in a beneficial manner (e.g., increased by at least 10%), or other clinically accepted symptoms or markers of disease (e.g., pain) are improved or ameliorated. Efficacy can also be measured by failure of a subject to worsen as assessed by hospitalization or need for medical interventions (e.g., progression of the disease is halted or at least slowed). Methods of measuring these indicators are known to those of skill in the art and/or described herein. Treatment includes any treatment of a disease in subject and includes: (1) inhibiting the disease, e.g., arresting, or slowing the progression of symptoms; or (2) relieving the disease, e.g., causing regression of symptoms; and (3) preventing or reducing the likelihood of the development of symptoms.

### IV. Kits for Therapeutic Use

The present disclosure also provides kits for use of the compositions described herein. For example, the present disclosure provides kits comprising a gene-editing system as described herein; a viral particle or set of viral particles comprising a gene-editing system as described herein; a nucleic acid or set of nucleic acids comprising a gene-editing system as described herein; and/or a population of genetically-edited cells as described herein.

In some embodiments, the kit can additionally comprise instructions for use in any of the methods described herein. The included instructions may comprise a description of: (i) the delivery of a gene-editing system as described herein; a viral particle or set of viral particles comprising a gene-editing system as described herein; and/or a nucleic acid or set of nucleic acids comprising a gene-editing system as described herein; and/or (ii) the administration of a population of genetically-edited cells as described herein.

The kit may further comprise a description of selecting a subject suitable for treatment based on identifying whether the subject is in need of the treatment. The instructions may include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1. Efficacy Screening of SpCas9 and SaCas9 gRNAs Targeted to SCN9A and SCN10A in iPSCs

Methods

### Guide RNA Design and Synthesis

In silico guide RNA design was completed by CRISPR Therapeutics. SpCas9 and SaCas9 guide RNAs targeting exons 2-15 of SCN9A and Exons 1-14 of SCN10A were designed in silico and evaluated using an off-target prediction algorithm. Guide RNAs with a favorable off-target profile were selected for synthesis and further on target evaluation. Selected gRNAs included 99 SpCas9 gRNAs (Table 1) and 68 SaCas9 gRNAs targeting SCN9A (Table 2) and 166 SpCas9 gRNAs (Table 3) and 73 SaCas9 gRNAs targeting SCN10A (Table 4).

Guide RNAs were custom ordered for synthesis by Synthego Corporation. Guide RNAs were ordered with standard chemical modifications which include 2′-O-methyl 3′ phosphorothioate modifications in the first and last 3 nucleotides. For SpCas9 gRNAs, the 20-nucleotide genome targeting sequences are listed in Tables 1 and 3, and a standard 80-mer SpCas9 scaffold sequence was added to create a guide RNA. For SaCas9 gRNAs, 22-nucleotide genome targeting sequences are listed in Tables 2 and 4 and were used for synthesis with the following SaCas9 scaffold sequence to generate guide RNAs: GUUUUAGUACUCUGGAAACAGAAUCUACUAAAACAA GGCAAAAUGCCGUGUUUAUCUCGUCAACUUGUUGGCGAGAUUUU (SEQ ID NO: 41).

Nucleofection of iPSCs using 4D-Nucleofector® System

Wildtype iPSCs, as well as engineered iPSCs stably expressing Cas9, were used for different steps of gRNA screening. iPSCs expressing SpCas9 or SaCas9 under the control of doxycycline were generated from wildtype iPSCs by inserting a targeting construct into the AAVS-1 locus. In this construct, two cassettes are expressed in opposing directions separated by an IS2 insulator element. The first expression cassette is a TetOn3G protein-2A-Puro under the control of the CASI promoter, and the second expression cassette is either SpCas9 or SaCas9 under the control of the TRE3G promoter.

iPSCs were electroporated using the Lonza 4D-Nucleofector® System together with the P3 Primary Cell 96-well Nucleofector™ Kit (Lonza, Cat: V4SP-3096) with program CM137. iPSCs were cultured in mTeSR1 (Stemcell Technologies, Cat: 85850). Prior to nucleofection cells were dissociated using Accutase (Stemcell Technologies, Cat: 07920) and resuspended in P3 Nucleofection solution. In 96-well format, 180,000 cells per well were electroporated with 400 ng Cas9 mRNA (TriLink) per well and 400 ng synthetic gRNA (Synthego) according to manufacturer's instructions. Following nucleofection iPSCs were maintained in mTeSR1 supplemented with 10 uM Y27632 (Stemcell Technologies, Cat: 72308) in 96 well cell culture plates pre-coated with matrigel for 72 hours prior to DNA extraction and next generation sequencing (NGS)-based insertion/deletion (Indel) detection. Two replicates were included in each electroporation experiment and two independent experiments were carried out. For stable SpCas9 and SaCas9 cell line experiments, cells were treated with 1 ug/ul doxycycline for 72 hours prior to Amaxa nucleofection.

Nucleofection of iPSC-Derived Sensory Neurons Using Lonza 4D-Nucleofector® Y Unit

To generate iPSC-derived sensory neuron cultures (iSNs), iPSC cells were differentiated in the presence of a cocktail of small molecule developmental pathway inhibitors in matrigel coated flasks. At DIV11 of differentiation, cells were dissociated and plated into 384 plates and maintained in maturation media which includes a cocktail of growth factors where they matured until DIV26-28. These neurons express canonical markers of nociceptors, including TRPV1, Brn3A, the peripheral marker Isl1, neuN and SCN9A (NaV1.7), and can recapitulate functional properties of physiologically relevant neuronal subtypes.

iPSC-derived sensory neurons (iSNs) were electroporated using the Lonza 4D-Nucleofector® Y Unit together with the AD1 4D-Nucleofector™ Y Kit (Lonza, Cat: V4YP-1A24) with program EH158. In 24-well format, cells were electroporated with ribonucleoprotein complexes (RNPs) according to manufacturer's instructions. RNP complexes were generated by incubating 425 pmol SpCas9 or SaCas9 protein (Aldevron) with 531 pmol synthetic gRNA (Synthego) at room temperature for 20 minutes. Following nucleofection iSNs were maintained in culture for 72 hours prior to DNA extraction and next generation sequencing (NGS)-based insertion/deletion (Indel) detection. Two replicates were included in each electroporation experiment and two independent experiments were carried out.

Transduction of iPSC-Derived Sensory Neurons with AAV

In 384-well format, approximately 12,000 iSNs per well were transduced with AAV-1 vectors expressing SaCas9 and a SaCas9 gRNA in a single vector. iSNs were transduced with AAV vectors at a multiplicity of infection (MOI) of 750,000. Following transduction, iSNs were maintained in culture for 7 days prior to DNA extraction and NGS based insertion/deletion (indel) detection. Two replicates were included in each transduction experiment and two independent experiments were carried out.

### Next Generation Sequencing (NGS) Based Insertion/Deletion (Indel) Detection

DNA was extracted from iPSCs 72 hours post electroporation using Lucigen Quick Extract 2×DNA Extraction Solution (Lucigen, Cat: QE09050) according to manufacturer's instructions. A two-step PCR approach using KAPA2G Robust HotStart ReadyMix (Sigma Aldrich, Cat: KK5702) was then used to generate NGS libraries. The first PCR was used to create amplicons while the second PCR was used to add Nextera DNA Index (i7/i5) adapter sequences. The reaction for PCR #1 comprised of 1 uL extracted gDNA, 1×KAPA2G Robust HotStart ReadyMix, 0.5 uM forward primer, and 0.5 uM reverse primer. Primer sequences are listed in Tables 5 and 6. The reaction for PCR #2 comprised of 1 ul PCR #1 product, 1×KAPA2G Robust HotStart ReadyMix, 0.5 uM Index 1 N7xx adapter, and 0.5 uM Index 2 N5xx adapter. Cycling conditions for both PCR #1 and PCR #2 were as follows: (1) 95° C. for 3 min, (2) 95° C. for 15 s, (3) 60° C. for 15 s, (4) 72° C. for 15 s, (5) repeat steps (2)-(4) 20 times, (6) 72° C. for 1 min, (7) 4° C. infinite hold. Samples were then pooled and purified using the Zymo DNA Clean and Concentrator Kit (Zymo, D4034) and quantified on the Agilent 2100 Bioanalyzer (Agilent, Cat: G2939BA). Then, libraries were run on Illumina's MiSeq to obtain paired-end reads (2×150).

For each sample, the reads were then filtered to obtain a minimum Phred33 quality score of 30. Paired-end reads were subsequently merged using FLASH (Fast Length Adjustment of SHort reads) with a requirement of at least 1 bp overlap. The resulting merged reads were then optimally aligned to the corresponding reference amplicon sequences using the Needleman-Wunsch algorithm. Reads that aligned with indels within 3 bp of the expected cut site were counted, and then filtered for frame-shifting indels only, where the indel length is not a multiple of 3. An estimate of total editing was calculated as the proportion of reads with indels proximal to the cut site, while productive editing was calculated as the proportion of reads with frame-shifting indel reads proximal to the cut site for each sample.

Once each sample was analyzed, quality control of the samples was then performed by requiring each sample to have at least 90% of sequenced reads successfully merged, and 70% of sequenced reads successfully aligned. Additionally, samples were required to have at least 1000 reads successfully aligned. Lastly, quality control was performed in a batch-aware manner, by dropping any samples whose final aligned read count was more than 2 standard deviations away from the mean of its corresponding batch of samples. Passing samples were averaged with the standard deviation calculated. Positive and negative controls were included, where the negative control was required to exhibited less than 2% indel rates, indicating a low level of background noise, and while the positive control samples had to show levels of editing above background. Further, reproducibility was confirmed by comparing corresponding samples between our two technical replicates; a strong linear fit was observed with a high R² of 0.85.

Off-Target Evaluation of SpCas9 gRNAs Targeted to SCN9A and SCN10A in iPSCs

For an initial off-target evaluation, an in silico nomination step was performed where candidate off-target sequences were predicted based on sequence similarity. Then, these sites were directly evaluated via targeted Next-Generation Sequencing to identify which sites, if any, showed evidence of CRISPR-Cas-induced off-target editing.

a) Computational Prediction of Off-Target Sites

Off-target sites were predicted based on sequence similarity using three computational algorithms. Specifically, CCTop and COSMID were each used to identify candidate off-target sites with up to 3 mismatches or up to 2 mismatches with 1 DNA or RNA bulge from the on-target sequence. The PAM sequence used for identifying off-target sites was NRG for SpCas9 guides and NNGRRT for SaCas9 guides. Guides identified from the two algorithms were then merged together, including de-duplication of sites with identical genomic coordinates. The total list of 1,471 putative off-target sites predicted across the 40 guides is provided in Table 7.

b) Hybrid Capture of iPSCs

iPSC transfections using two different wildtype donors were performed using the Lonza conditions described above. Two biological replicates were used, and genomic DNA pooled to obtain the necessary amount for hybrid capture. DNA was extracted from iPSCs 72 hours post electroporation using the DNeasy 96 Blood and Tissue Kit (Qiagen, Cat: 69581). Samples were quantified using the Qubit 1×dsDNA HS Assay (ThermoFisher, Cat: Q33231) and EnVision plate reader with 4PL calculation. A minimum of 200 ng of each sample was obtained and processed for hybrid capture using the SureSelect XT Reagent Kit (Aglient, Cat: G9704A). Briefly, samples were fragmented to 150-200 bp using the Covaris LE220 and end repaired, dA-tailed, and adapter ligated. Libraries were then amplified using Herculase II Fusion DNA polymerase using the following cycle conditions: (1) 98° C. for 2 min, (2) 98° C. for 30 s, (3) 65° C. for 30 s, (4) 72° C. for 1 min, (5) repeat steps (2)-(4) 10 times, (6) 72° C. for 5 min, (7) 4° C. infinite hold. A bead-based clean-up was used to purify libraries, AMPure XP (Beckman Coulter, Cat: A63881). Libraries were hybridized to the target-specific Capture Library and target molecules captures with Steptavidin-coated magnetic beads. Captured libraries were then amplified and purified using the same conditions above. Samples were QC'd using the DNA High Sensitivity kits on the TapeStation (Agilent, Cat: 5067-5584) and/or Bioanalyer (Aglient, Cat: 5067-1504) and sequenced on the Illumina HiSeq platform to a median sequencing coverage of 2,272× per candidate off-target site.

c) Computational Analysis of Targeted Next-Generation Sequencing

For each putative off-target site included in this study, the following analysis was performed to determine strength of evidence for CRISPR-Cas-treatment-induced off-target editing. First, next-generation sequencing reads were aligned to the hg38 human reference genome using the alignment tool bwa in the mem mode with default parameters, followed by conversion and sorting of the SAM and BAM files with read duplicate removal performed by samtools. Then, the indel formation rate was measured by piling up reads with an indel within 3 bp of the expected cut site using the Python package pysam and dividing the number of indel reads by the total number of reads covering that site.

Then, the indel rate measured at each predicted off-target site was compared between the treated sample of each iPSC donor and the untreated (electroporated only) negative control sample matched for that same iPSC donor. If an indel rate at the site was observed to be >0.2% greater than the negative control sample, the data for that candidate site entered statistical testing. The only exception was for candidate sites that were observed to have a germline indel genetic variant, where an indel rate of ˜50% or ˜100% is observed in both the matched untreated and treated sample of one or more donors. For sites that entered statistical testing, a paired t-test was performed across both donors for the treated and untreated indel rates at that site. Any tested site that resulted in a p-value of less than 0.05 was considered to have confirmed off-target editing. Substantial on-target editing (average indel rate of 9.35%-52.25%) was confirmed across guides as a positive control for this study.

TABLE 1
Names and sequences of SpCas9 guide RNAs
targeted to the SCN9A gene (Nav1.7)
Nav1.7 SpCas9 gRNAs

| SEQ | | spacer sequence |
|---|---|---|
| ID NO: | gRNA Name | (5′-3′) |
| 43 | Scn9a Sp Exon 2_T1 | AuCuAuGGGGACAuuCCuCC |
| 44 | Scn9a Sp Exon 2_T2 | CAGCAAuGCGuuGuuCAAuG |
| 45 | Scn9a Sp Exon 2_T3 | AGuAGGGGuCCAAGuCCuCC |
| 46 | Scn9a Sp Exon 2_T4 | uGGGGACAuuCCuCCCGGCA |
| 47 | Scn9a Sp Exon 2_T5 | GuAGGGGuCCAAGuCCuCCA |
| 48 | Scn9a Sp Exon 2_T6 | uAGGGGuCCAAGuCCuCCAG |
| 49 | Scn9a Sp Exon 2_T7 | AuGGCAAuGuuGCCuCCCCC |
| 50 | Scn9a Sp Exon 2_T8 | GGCuCuGACACCAuGCCGGG |
| 51 | Scn9a Sp Exon 2_T9 | AACAGCuGCCCuuCAuCuAu |
| 8 | Scn9a Sp Exon 2_T10 | GGAAuGuCCCCAuAGAuGAA |
| 52 | Scn9a Sp Exon 2_T11 | CGGCAuGGuGuCAGAGCCCC |
| 53 | Scn9a Sp Exon 2_T12 | AGCAAuGCGuuGuuCAAuGA |
| 54 | Scn9a Sp Exon 2_T13 | AGGAAuGuCCCCAuAGAuGA |
| 55 | Scn9a Sp Exon 2_T14 | AAACAGCuGCCCuuCAuCuA |
| 56 | Scn9a Sp Exon 2_T15 | CCCCuACuAuGCAGACAAAA |
| 57 | Scn9a Sp Exon 4_T1 | CCAuGAAuAACCCACCGGAC |
| 58 | Scn9a Sp Exon 4_T2 | CAuuuuuGGuCCAGuCCGGu |
| 59 | Scn9a Sp Exon 4_T3 | CCAGuCCGGuGGGuuAuuCA |
| 60 | Scn9a Sp Exon 4_T4 | ACAuuuuuGGuCCAGuCCGG |
| 61 | Scn9a Sp Exon 4_T5 | uCGACAuuuuuGGuCCAGuC |
| 62 | Scn9a Sp Exon 4_T6 | ACCCACuuACuCGACAuuuu |
| 63 | Scn9a Sp Exon 4_T7 | uAuGACCAuGAAuAACCCAC |
| 64 | Scn9a Sp Exon 5_T1 | uuCuuCGuGACCCGuGGAAC |
| 65 | Scn9a Sp Exon 5_T2 | uCGuGACCCGuGGAACuGGC |
| 66 | Scn9a Sp Exon 5_T3 | uCACuuuuCuuCGuGACCCG |
| 67 | Scn9a Sp Exon 5_T4 | GuGuuuAGGuACACuuuuAC |
| 68 | Scn9a Sp Exon 7_T1 | AAGCCCCuACAAuuGuCuuC |
| 69 | Scn9a Sp Exon 7_T2 | CuGAGuGuGuuuGCACuAAu |
| 70 | Scn9a Sp Exon 7_T3 | AuuGGACuACAGCuGuuCAu |
| 7 | Scn9a Sp Exon 7_T5 | CAGGCCuGAAGACAAuuGuA |
| 71 | Scn9a Sp Exon 7_T6 | AGGCCuGAAGACAAuuGuAG |
| 72 | Scn9a Sp Exon 9_T1 | AAGCuCGuGuAGCCAuAAuC |
| 73 | Scn9a Sp Exon 9_T2 | AGCuCGuGuAGCCAuAAuCA |
| 74 | Scn9a Sp Exon 9_T3 | GGCuAAuGACCCAAGAuuAC |
| 75 | Scn9a Sp Exon 9_T4 | uuCACACAGGuGuACCCCuC |
| 76 | Scn9a Sp Exon 9_T5 | CGuGuGuAGuCAGuGuCCAG |
| 77 | Scn9a Sp Exon 9_T6 | GCuAAuGACCCAAGAuuACu |
| 78 | Scn9a Sp Exon 9_T7 | CGAGCuuuGACACuuuCAGC |
| 79 | Scn9a Sp Exon 9_T8 | uGGuACuCACCuGuuGGuAA |
| 80 | Scn9a Sp Exon 9_T9 | GGGuACACCuGuGuGAAAAu |
| 81 | Scn9a Sp Exon 9_T10 | CuGGGAAAACCuuuACCAAC |
| 82 | Scn9a Sp Exon 9_T11 | uuGGGuCAuuAGCCuAAACA |
| 83 | Scn9a Sp Exon 9_T12 | GuGuGuAGuCAGuGuCCAGA |
| 84 | Scn9a Sp Exon 9_T13 | GGGCCuuCuuAGCCuuGuuu |
| 85 | Scn9a Sp Exon 9_T14 | GAGCuuuGACACuuuCAGCu |
| 86 | Scn9a Sp Exon 9_T15 | AuuGGCAGAAACCCuGAuuA |
| 87 | Scn9a Sp Exon 10_T1 | CCCuAGACGCuGCGuGCuGC |
| 88 | Scn9a Sp Exon 10_T2 | CCAGCAGCACGCAGCGuCuA |
| 89 | Scn9a Sp Exon 10_T4 | GCCAGCAGCACGCAGCGuCu |
| 90 | Scn9a Sp Exon 10_T5 | CuuuGuCGuAGuGAuuuuCC |
| 91 | Scn9a Sp Exon 11_T2 | GAGGuuGuCuACCCCCAAuC |
| 92 | Scn9a Sp Exon 11_T3 | uAuGCCCuuCGACACCAAGG |
| 93 | Scn9a Sp Exon 11_T5 | ACCuuGGuGuCGAAGGGCAu |
| 3 | Scn9a Sp Exon 11_T6 | GCCuAuGCCCuuCGACACCA |
| 94 | Scn9a Sp Exon 11_T7 | AGuuuCCACCuuGGuGuCGA |
| 1 | Scn9a Sp Exon 11_T9 | CAAuuuGGGuGGuACCuGAu |
| 95 | Scn9a Sp Exon 11_T10 | GuuuCCACCuuGGuGuCGAA |
| 96 | Scn9a Sp Exon 11_T11 | CCGCuGCCGCuGCAAuuGCC |
| 97 | Scn9a Sp Exon 11_T12 | GCCCAACCAGGCAAuuGCAG |
| 5 | Scn9a Sp Exon 11_T13 | CGGCuGAAuAuACAAGuAuu |
| 4 | Scn9a Sp Exon 11_T14 | AuAGGCGAGCACAuGAAAAG |
| 98 | Scn9a Sp Exon 11_T15 | AAuuuGGGuGGuACCuGAuu |
| 99 | Scn9a Sp Exon 12_T1 | CGuuCACCGGCAGCAuuGGu |
| 100 | Scn9a Sp Exon 12_T2 | CCGuuCACCGGCAGCAuuGG |
| 101 | Scn9a Sp Exon 12_T3 | uGuuACuGCuGCGuCGCuCC |
| 102 | Scn9a Sp Exon 12_T4 | GuuACuGCuGCGuCGCuCCu |
| 103 | Scn9a Sp Exon 12_T5 | GGGCuGAGCGuCCAuCAACC |
| 104 | Scn9a Sp Exon 12_T6 | CACCAAuGCuGCCGGuGAAC |
| 491 | Scn9a Sp Exon 12_T7 | uuCCCGuuCACCGGCAGCAu |
| 105 | Scn9a Sp Exon 12_T8 | GGCuGAGCGuCCAuCAACCA |
| 492 | Scn9a Sp Exon 12_T9 | GuuCACCGGCAGCAuuGGuG |
| 106 | Scn9a Sp Exon 12_T10 | uuACuGCuGCGuCGCuCCuG |
| 9 | Scn9a Sp Exon 12_T11 | CCACCAAuGCuGCCGGuGAA |
| 107 | Scn9a Sp Exon 12_T12 | CuGCAACGGuGuGGuCuCCC |
| 108 | Scn9a Sp Exon 12_T13 | CAGCAuuGGuGGGGACCuAC |
| 493 | Scn9a Sp Exon 12_T14 | uuGGuGGGGACCuACuGGCu |
| 109 | Scn9a Sp Exon 12_T16 | GCGuCGCuCCuGGGGuCuGu |
| 10 | Scn9a Sp Exon 12_T17 | CAGuCACCACuCAGCAuuCG |
| 494 | Scn9a Sp Exon 12_T18 | uAGGuCCCCACCAAuGCuGC |
| 110 | Scn9a Sp Exon 12_T19 | uGCuGuGGACuGCAACGGuG |
| 111 | Scn9a Sp Exon 12_T20 | CGuCGCuCCuGGGGuCuGuG |
| 112 | Scn9a Sp Exon 12_T21 | uGCGuCGCuCCuGGGGuCuG |
| 113 | Scn9a Sp Exon 12_T22 | GGuGuGGuCuCCCuGGuuGA |
| 114 | Scn9a Sp Exon 12_T23 | GuAACAuCAGCCAAGCCAGu |
| 115 | Scn9a Sp Exon 12_T24 | CuGuGCAuuuuCCCGuuCAC |
| 2 | Scn9a Sp Exon 12_T25 | GCuuCGCCuuGCAGAAAACA |
| 116 | Scn9a Sp Exon 12_T26 | GuuuGuGCCCCACAGACCCC |
| 495 | Scn9a Sp Exon 12_T27 | GCuGuCCAuuGGGGAGCAuG |
| 496 | Scn9a Sp Exon 12_T28 | uCuGGCAGAAGCuGuCCAuu |
| 6 | Scn9a Sp Exon 14_T1 | GGAACACCACCCAAuGACuG |
| 117 | Scn9a Sp Exon 14_T2 | GCAAAuCuGuACCACCAAGG |
| 118 | Scn9a Sp Exon 14_T3 | uGuGCAAAuCuGuACCACCA |
| 119 | Scn9a Sp Exon 14_T4 | CCAAGGuGGACAuuuuuGuC |
| 120 | Scn9a Sp Exon 14_T5 | uGuCuGGACuCuuCAAGuuC |
| 121 | Scn9a Sp Exon 14_T6 | uCuGGAAuuGCuCuCCAuAu |
| 122 | Scn9a Sp Exon 15_T1 | AGuuCuGCGAuCAuuCAGAC |
| 123 | Scn9a Sp Exon 15_T2 | GGAGCuCuuuCuAGCAGAuG |
| 124 | Scn9a Sp Exon 15_T3 | CCuACuuGGAAAuACuCAuA |

TABLE 2
Names and sequences of SaCas9 guide RNAs
targeted to the SCN9A gene (Nav1.7)
Nav1.7 SaCas9 gRNAs

| SEQ | | |
|---|---|---|
| ID NO: | gRNA Name | spacer sequence (5′-3′) |
| 125 | Scn9a Sa Exon 2_T1 | GGGGCuCuGACACCAuGCCGGG |
| 126 | Scn9a Sa Exon 2_T2 | CCCCuACuAuGCAGACAAAAAG |
| 127 | Scn9a Sa Exon 2_T3 | CuCACCuuuuuGuCuGCAuAGu |
| 128 | Scn9a Sa Exon 2_T4 | AuCAuCAuCuuuCuuuuCuuCu |
| 129 | Scn9a Sa Exon 3_T1 | uuuCuCCuuuCAGuCCuCuAAG |
| 130 | Scn9a Sa Exon 4_T1 | uCGACAuuuuuGGuCCAGuCCG |
| 131 | Scn9a Sa Exon 4_T4 | CCACCGGACuGGACCAAAAAuG |
| 132 | Scn9a Sa Exon 4_T5 | GACAAACuGCAuAuuuAuGACC |
| 133 | Scn9a Sa Exon 4_T6 | AAuAGuGCACAuGAuGAGCAuG |
| 134 | Scn9a Sa Exon 4_T7 | uGGuCAuAAAuAuGCAGuuuGu |
| 135 | Scn9a Sa Exon 5_T1 | CuCCuACACAGAAGCCuCuuGC |
| 136 | Scn9a Sa Exon 5_T2 | uCuuCGuGACCCGuGGAACuGG |
| 137 | Scn9a Sa Exon 5_T3 | GuuCCACGGGuCACGAAGAAAA |
| 138 | Scn9a Sa Exon 5_T4 | CuuGCAAGAGGCuuCuGuGuAG |
| 139 | Scn9a Sa Exon 5_T5 | uuGuGuuuAGGuACACuuuuAC |
| 13 | Scn9a Sa Exon 5_T6 | ACGACAAAAuCCAGCCAGuuCC |
| 140 | Scn9a Sa Exon 5_T7 | ACuuuuACuGGAAuAuAuACuu |
| 141 | Scn9a Sa Exon 6_T2 | uACAAAuuCuGuuAAAuACCuG |
| 142 | Scn9a Sa Exon 6_T5 | AuuuAAuuCuACAGGuAuuuAA |
| 17 | Scn9a Sa Exon 7_T1 | CAuGAuCCuGACuGuGuuCuGu |
| 143 | Scn9a Sa Exon 7_T2 | uuCuAAAGuCuuCuuCACuCuC |
| 144 | Scn9a Sa Exon 7_T4 | GAGuGAAGAAGACuuuAGAAGu |
| 145 | Scn9a Sa Exon 7_T5 | AGAAAGCAuAAuGAAuACCCuA |
| 146 | Scn9a Sa Exon 7_T6 | ACACACuCAGACAGAACACAGu |
| 147 | Scn9a Sa Exon 7_T7 | uAAuGAAACAuuAGAAAGCAuA |
| 148 | Scn9a Sa Exon 7_T8 | uCuAAuGuuuCAuuAuuuuCAA |
| 149 | Scn9a Sa Exon 8_T1 | GAAACCACAAAGGAGAGCAuCu |
| 150 | Scn9a Sa Exon 8_T2 | CuuuGuGGuuuCAGCACAGAuu |
| 151 | Scn9a Sa Exon 8_T3 | ACAGAAuAuuuuuAuuACuuGG |
| 152 | Scn9a Sa Exon 9_T1 | uCAAAGCuCGuGuAGCCAuAAu |
| 18 | Scn9a Sa Exon 9_T2 | CuCGuGuGuAGuCAGuGuCCAG |
| 14 | Scn9a Sa Exon 9_T3 | CuGGGAAAACCuuuACCAACAG |
| 153 | Scn9a Sa Exon 9_T4 | GGuAAAGGuuuuCCCAGuAAuC |
| 154 | Scn9a Sa Exon 10_T1 | AACAuuGAAGAAGCuAAACAGA |
| 155 | Scn9a Sa Exon 10_T2 | CAuAuGCCAuGGCAACCACAGC |
| 156 | Scn9a Sa Exon 10_T3 | GAAGAAGCuAAACAGAAAGAAu |
| 157 | Scn9a Sa Exon 11_T1 | GCAAuuuGGGuGGuACCuGAuu |
| 158 | Scn9a Sa Exon 11_T2 | CAGGCAAuuGCAGCGGCAGCGG |
| 11 | Scn9a Sa Exon 11_T3 | AAGCAGAAuuAuGGGCCuCuCA |
| 159 | Scn9a Sa Exon 11_T4 | uuuAGCACuuuuAGAGCuCAGu |
| 160 | Scn9a Sa Exon 11_T5 | GAuGCuGAGAAAuuGuCGAAAu |
| 161 | Scn9a Sa Exon 11_T6 | AAuAuACAAGuAuuAGGAGAAG |
| 16 | Scn9a Sa Exon 12_T1 | GAuGuuACuGCuGCGuCGCuCC |
| 12 | Scn9a Sa Exon 12_T2 | GCCuuGCAGAAAACAAGGAGCC |
| 20 | Scn9a Sa Exon 12_T3 | AGAAAACAAGGAGCCACGAAuG |
| 162 | Scn9a Sa Exon 12_T4 | GGAAGAGAuAuAGGAuCuGAGA |
| 163 | Scn9a Sa Exon 12_T5 | uuCAAAGGCAGAGGAAGAGAuA |
| 164 | Scn9a Sa Exon 13_T1 | CuAuCuCCuuuCAGAGGAuAuG |
| 165 | Scn9a Sa Exon 13_T2 | CuCAuuGCuCuCuGuCuGAGGu |
| 15 | Scn9a Sa Exon 13_T3 | uCCCAACCuCAGACAGAGAGCA |
| 166 | Scn9a Sa Exon 13_T4 | uuGuAGuuCCuAuCuCCuuuCA |
| 167 | Scn9a Sa Exon 14_T1 | AuGGAACACCACCCAAuGACuG |
| 168 | Scn9a Sa Exon 14_T2 | AuAuGGAGAGCAAuuCCAGAuC |
| 169 | Scn9a Sa Exon 14_T3 | uGAuCuGGAAuuGCuCuCCAuA |
| 170 | Scn9a Sa Exon 14_T4 | AuGGuAAuuGCAAGAuCuACAA |
| 171 | Scn9a Sa Exon 14_T5 | uGCuuuuuuCuCCCAGAACuuG |
| 172 | Scn9a Sa Exon 14_T6 | AuuuCCuAuAGCAAGuACAuuu |
| 173 | Scn9a Sa Exon 14_T7 | ACAuuuuuGAAuuCCuCAGuCA |
| 174 | Scn9a Sa Exon 14_T8 | AuuuGCACACAAAuuCuuGAuC |
| 175 | Scn9a Sa Exon 14_T9 | AAAGuGuAuCuAuuuuAuuGuA |
| 176 | Scn9a Sa Exon 14_T10 | uACAAuAAAAuAGAuACACuuu |
| 177 | Scn9a Sa Exon 15_T1 | AAuGGuAuuAAAACuGAuuGCC |
| 178 | Scn9a Sa Exon 15_T2 | AuAuGAGuAuuuCCAAGuAGGC |
| 19 | Scn9a Sa Exon 15_T3 | AAACuGAuuGCCAuGGAuCCAu |
| 179 | Scn9a Sa Exon 15_T4 | ACCuuAGuuuAuGuuuACCAGu |
| 180 | Scn9a Sa Exon 15_T5 | CAGCCuACuuGGAAAuACuCAu |
| 181 | Scn9a Sa Exon 15_T6 | GAGCuCuuuCuAGCAGAuGuGG |
| 182 | Scn9a Sa Exon 15_T7 | uuuuuCuCACuuAGGuCuuuAC |

TABLE 3
Names and sequences of SpCas9 guide RNAs targeted
to the SCN10A gene (Nav1.8)
Nav1.8 SpCas9 gRNAs

| SEQ | | |
|---|---|---|
| ID NO: | gRNA Name | spacer sequence (5′-3′) |
| 183 | Scn10a Sp Exon 1_T2 | GACGGAAGuuGuuAGuuuCG |
| 184 | Scn10a Sp Exon 1_T3 | CAACuuCCGuCGCuuuACuC |
| 185 | Scn10a Sp Exon 1_T4 | uCGCuuuACuCCGGAGuCAC |
| 186 | Scn10a Sp Exon 1_T5 | GAACGGAuCuAGAuCCuCCA |
| 187 | Scn10a Sp Exon 1_T6 | ACGGAAGuuGuuAGuuuCGA |
| 188 | Scn10a Sp Exon 1_T7 | AACGGAuCuAGAuCCuCCAG |
| 189 | Scn10a Sp Exon 1_T8 | AGAACGGAuCuAGAuCCuCC |
| 190 | Scn10a Sp Exon 1_T9 | GuGACuCCGGAGuAAAGCGA |
| 191 | Scn10a Sp Exon 1_T10 | uuAGuuuCGAGGGAuCCAAu |
| 192 | Scn10a Sp Exon 1_T11 | GuuAGuuuCGAGGGAuCCAA |
| 193 | Scn10a Sp Exon 1_T12 | GGCuCCCCGAuCAGuuCuGC |
| 194 | Scn10a Sp Exon 1_T14 | uAGuuuCGAGGGAuCCAAuG |
| 21 | Scn10a Sp Exon 1_T15 | GCuCCCCGAuCAGuuCuGCu |
| 195 | Scn10a Sp Exon 1_T16 | GCuCCCAGCAGAACuGAuCG |
| 28 | Scn10a Sp Exon 1_T17 | AuCCGuuCuACAGCACACAC |
| 196 | Scn10a Sp Exon 1_T18 | ACCCGGuGuGuGCuGuAGAA |
| 197 | Scn10a Sp Exon 1_T19 | AGAACuGAuCGGGGAGCCCC |
| 198 | Scn10a Sp Exon 1_T20 | uCCGuuCuACAGCACACACC |
| 199 | Scn10a Sp Exon 1_T21 | CuuuACuCCGGAGuCACuGG |
| 200 | Scn10a Sp Exon 1_T22 | CACCAuAGAACuuGGGCAGC |
| 201 | Scn10a Sp Exon 1_T23 | uGGGAGCuCACCAuAGAACu |
| 202 | Scn10a Sp Exon 1_T24 | ACuGAuCGGGGAGCCCCuGG |
| 203 | Scn10a Sp Exon 1_T25 | AACCAGCuGCCCAAGuuCuA |
| 204 | Scn10a Sp Exon 1_T26 | GAACuuGGGCAGCuGGuuGC |
| 205 | Scn10a Sp Exon 1_T27 | GAAGCAAAuuGCuGCCAAGC |
| 206 | Scn10a Sp Exon 1_T28 | uCuAuCuCCACCAGuGACuC |
| 207 | Scn10a Sp Exon 1_T29 | GGGGCCGAGGCuuCuCuuCu |
| 26 | Scn10a Sp Exon 1_T30 | GAGCuCCCAGCAGAACuGAu |
| 208 | Scn10a Sp Exon 2_T1 | GGGCCCGAGuGGCACuAAAC |
| 209 | Scn10a Sp Exon 2_T2 | uuCCCGGuuuAGuGCCACuC |
| 210 | Scn10a Sp Exon 2_T3 | GGCCCGAGuGGCACuAAACC |
| 211 | Scn10a Sp Exon 2_T4 | uuuCCCGGuuuAGuGCCACu |
| 212 | Scn10a Sp Exon 2_T5 | uuAGuGCCACuCGGGCCCuG |
| 213 | Scn10a Sp Exon 2_T6 | uGAuGGCCGuuCuuCuGAuC |
| 214 | Scn10a Sp Exon 2_T7 | GAAuAGCCACAGGGCCCGAG |
| 215 | Scn10a Sp Exon 2_T8 | GuuCuuCuGAuCAGGuuGAA |
| 216 | Scn10a Sp Exon 2_T9 | AGuGGCACuAAACCGGGAAA |
| 217 | Scn10a Sp Exon 2_T10 | ACAAAGGGAGGACCAuuuCC |
| 218 | Scn10a Sp Exon 4_T1 | GGAuuuuAGCGuCAuuACCC |
| 29 | Scn10a Sp Exon 4_T2 | uCACGuACCuGAGAGAuCCu |
| 219 | Scn10a Sp Exon 4_T3 | CAuAGAGAuAuACuCACGCC |
| 220 | Scn10a Sp Exon 4_T4 | GCCAGuuCCAAGGAuCuCuC |
| 221 | Scn10a Sp Exon 4_T5 | ACCuGAGAGAuCCuuGGAAC |
| 222 | Scn10a Sp Exon 5_T1 | GCACAGCAAuAGAuCuCCGu |
| 223 | Scn10a Sp Exon 5_T2 | uAAGAACuCuGAAuGuCCGC |
| 224 | Scn10a Sp Exon 5_T3 | AuAGAuCuCCGuGGGAuCuC |
| 225 | Scn10a Sp Exon 5_T4 | GGCACAGCAAuAGAuCuCCG |
| 226 | Scn10a Sp Exon 6_T2 | GGGCCCCCACAAuGACCuuC |
| 227 | Scn10a Sp Exon 6_T3 | CGCAGGCCuGAAGGuCAuuG |
| 228 | Scn10a Sp Exon 6_T4 | GuGGGGCuGCAACuCuuCAA |
| 229 | Scn10a Sp Exon 6_T7 | GCAGGCCuGAAGGuCAuuGu |
| 230 | Scn10a Sp Exon 6_T8 | GGuGGGGCuGCAACuCuuCA |
| 231 | Scn10a Sp Exon 6_T9 | uCAuCuuCCCGCAGGCCuGA |
| 232 | Scn10a Sp Exon 6_T10 | GAAGAGuuGCAGCCCCACCA |
| 233 | Scn10a Sp Exon 7_T1 | GACCCCuuACuGuGuGGCAA |
| 234 | Scn10a Sp Exon 7_T2 | AGAuCCAuuGCCACACAGuA |
| 235 | Scn10a Sp Exon 7_T3 | uGuGGCAAuGGAuCuGACuC |
| 236 | Scn10a Sp Exon 7_T4 | GuGGCAAuGGAuCuGACuCA |
| 237 | Scn10a Sp Exon 7_T5 | GAuCCAuuGCCACACAGuAA |
| 25 | Scn10a Sp Exon 7_T6 | ACuuCuGACCCCuuACuGuG |
| 238 | Scn10a Sp Exon 7_T7 | AuCCAuuGCCACACAGuAAG |
| 239 | Scn10a Sp Exon 8_T1 | ACuGuuCCGCCuCAuGACAC |
| 240 | Scn10a Sp Exon 8_T2 | CuGGGAACGCCuCuACCAGC |
| 241 | Scn10a Sp Exon 8_T3 | CCGGGuuGuCAGAAGuuuuA |
| 242 | Scn10a Sp Exon 8_T4 | GCCuCAuGACACAGGAuuCC |
| 243 | Scn10a Sp Exon 8_T5 | AGCuCAGuACCuGCuGGuAG |
| 244 | Scn10a Sp Exon 8_T6 | uuAAGGCAGAuAuAACCAuC |
| 245 | Scn10a Sp Exon 8_T7 | AAGCuGGuGuAGuuAAAAuC |
| 246 | Scn10a Sp Exon 8_T8 | uCAuGAGGCGGAACAGuGAG |
| 247 | Scn10a Sp Exon 8_T9 | CCuuAAAACuuCuGACAACC |
| 248 | Scn10a Sp Exon 8_T10 | GAAuGCAGCuCAGuACCuGC |
| 249 | Scn10a Sp Exon 9_T1 | GGCCCuCGAGAuGCuCCGGA |
| 22 | Scn10a Sp Exon 9_T2 | uGuAGuCACCAuGGCGuAuG |
| 250 | Scn10a Sp Exon 9_T3 | GuuCuGCuCCuCAuACGCCA |
| 251 | Scn10a Sp Exon 9_T4 | CuCCuuCCGGAGCAuCuCGA |
| 252 | Scn10a Sp Exon 9_T5 | CuACCuGGuCAACuuGAuCu |
| 253 | Scn10a Sp Exon 9_T6 | GCuCCuuCCGGAGCAuCuCG |
| 254 | Scn10a Sp Exon 9_T7 | CGAGAuGCuCCGGAAGGAGC |
| 255 | Scn10a Sp Exon 9_T8 | AGGAGGCCCuCGAGAuGCuC |
| 256 | Scn10a Sp Exon 9_T9 | uuuGuGCuCGuAAuCuuCCu |
| 257 | Scn10a Sp Exon 9_T10 | GGAGCAuCuCGAGGGCCuCC |
| 258 | Scn10a Sp Exon 9_T11 | GGCGuAuGAGGAGCAGAACC |
| 259 | Scn10a Sp Exon 9_T12 | uuuuGuGCuCGuAAuCuuCC |
| 260 | Scn10a Sp Exon 9_T13 | uGACCAGGuAGAAAGAuCCC |
| 261 | Scn10a Sp Exon 9_T14 | GAuCuuGGCuGuAGuCACCA |
| 262 | Scn10a Sp Exon 10_T1 | ACCAuCCuGCGCuGGuuGuA |
| 263 | Scn10a Sp Exon 10_T2 | CuGAuCCuuACAACCAGCGC |
| 30 | Scn10a Sp Exon 10_T3 | CGCAGGuGCuAGCAGCACuA |
| 264 | Scn10a Sp Exon 10_T4 | uCGCAGGuGCuAGCAGCACu |
| 265 | Scn10a Sp Exon 10_T5 | GGuuAAAGGuGAuCCAuuGu |
| 266 | Scn10a Sp Exon 10_T6 | AGCCuCuuACCAuCCuGCGC |
| 24 | Scn10a Sp Exon 10_T7 | uCCuuACAACCAGCGCAGGA |
| 267 | Scn10a Sp Exon 10_T9 | AGGuuAAAGGuGAuCCAuuG |
| 268 | Scn10a Sp Exon 10_T10 | uGGuuGuAAGGAuCAGAGCG |
| 269 | Scn10a Sp Exon 10_T13 | GuGGAGCCCuCuGACACuCu |
| 270 | Scn10a Sp Exon 11_T1 | GCuCACuAGuGGGCGGCGGu |
| 271 | Scn10a Sp Exon 11_T2 | GGAAAACGCCGGGCuAGuCA |
| 272 | Scn10a Sp Exon 11_T3 | ACuAGCCCGGCGuuuuCCAG |
| 273 | Scn10a Sp Exon 11_T4 | ACCGAGACAuCGACAGCuCC |
| 274 | Scn10a Sp Exon 11_T5 | CCCGGCGuuuuCCAGAGGCG |
| 275 | Scn10a Sp Exon 11_T6 | GAGAGCCCCGAuGGCuuuCG |
| 276 | Scn10a Sp Exon 11_T7 | CGAuGGCuuuCGuGGuCuCC |
| 497 | Scn10a Sp Exon 11_T8 | GCAAGCuCACuAGuGGGCGG |
| 277 | Scn10a Sp Exon 11_T9 | CCuCGCCuCuGGAAAACGCC |
| 27 | Scn10a Sp Exon 11_T10 | CCGAGACAuCGACAGCuCCA |
| 278 | Scn10a Sp Exon 11_T11 | CGAGACAuCGACAGCuCCAG |
| 279 | Scn10a Sp Exon 11_T12 | GCCuCGCCuCuGGAAAACGC |
| 280 | Scn10a Sp Exon 11_T13 | GGGAGuGAGAuAuCuCGGCC |
| 281 | Scn10a Sp Exon 11_T14 | GGAGACCACGAAAGCCAuCG |
| 282 | Scn10a Sp Exon 11_T15 | CGAGAuAuCuCACuCCCuGA |
| 283 | Scn10a Sp Exon 11_T16 | CCCACuAGuGAGCuuGCCCC |
| 284 | Scn10a Sp Exon 11_T17 | CCAGGGGCAAGCuCACuAGu |
| 285 | Scn10a Sp Exon 11_T18 | GAGuGAGAuAuCuCGGCCAG |
| 286 | Scn10a Sp Exon 11_T19 | CCGAGAuAuCuCACuCCCuG |
| 287 | Scn10a Sp Exon 11_T20 | CuCGGCCAGGGGACCGGAAA |
| 288 | Scn10a Sp Exon 11_T21 | AGAuAuCuCGGCCAGGGGAC |
| 289 | Scn10a Sp Exon 11_T22 | GuGuuCCAuuuCCGGuCCCC |
| 290 | Scn10a Sp Exon 11_T23 | uCCAGGGGCAAGCuCACuAG |
| 291 | Scn10a Sp Exon 11_T24 | GGGGCAAGCuCACuAGuGGG |
| 292 | Scn10a Sp Exon 11_T25 | GGAGuGAGAuAuCuCGGCCA |
| 293 | Scn10a Sp Exon 11_T26 | uGGAGACCACGAAAGCCAuC |
| 294 | Scn10a Sp Exon 11_T27 | GGCGAGGCCuAGAAAAGACu |
| 295 | Scn10a Sp Exon 11_T28 | CCCuGGAGCuGuCGAuGuCu |
| 296 | Scn10a Sp Exon 11_T29 | CuGGAGACCACGAAAGCCAu |
| 297 | Scn10a Sp Exon 11_T30 | uCuuuuCuAGGCCuCGCCuC |
| 298 | Scn10a Sp Exon 11_T31 | AGGCGAGGCCuAGAAAAGAC |
| 299 | Scn10a Sp Exon 11_T32 | CCuCAGGGAGuGAGAuAuCu |
| 300 | Scn10a Sp Exon 11_T33 | GuuGAGGAAGAGGGCuuCuA |
| 301 | Scn10a Sp Exon 11_T34 | uuCuAGGGAGGGGGCCuuGC |
| 302 | Scn10a Sp Exon 12_T1 | uCuCAACAGGCAuuCGAuGC |
| 303 | Scn10a Sp Exon 12_T2 | AuGAACCuuuCCGGGCCCAA |
| 304 | Scn10a Sp Exon 12_T3 | GCACuuACCCuCAAGGACGG |
| 305 | Scn10a Sp Exon 12_T4 | uAuCAuAACCuCCGuCCuuG |
| 306 | Scn10a Sp Exon 12_T5 | uGAACCuuuCCGGGCCCAAA |
| 307 | Scn10a Sp Exon 12_T6 | AuCAuAACCuCCGuCCuuGA |
| 308 | Scn10a Sp Exon 12_T7 | AuuGCCCuuuGGGCCCGGAA |
| 309 | Scn10a Sp Exon 12_T8 | GuGAGCAGCACuuACCCuCA |
| 310 | Scn10a Sp Exon 12_T9 | GCAGCACuuACCCuCAAGGA |
| 311 | Scn10a Sp Exon 12_T10 | CACuCAuuGCCCuuuGGGCC |
| 312 | Scn10a Sp Exon 12_T12 | GACAACACuCAuuGCCCuuu |
| 313 | Scn10a Sp Exon 12_T13 | AuACuuAGAuGAACCuuuCC |
| 314 | Scn10a Sp Exon 12_T14 | uGACAACACuCAuuGCCCuu |
| 315 | Scn10a Sp Exon 13_T1 | CACuCACGAuGuuGCCuAuC |
| 316 | Scn10a Sp Exon 13_T4 | uuCGAAGCCAuGCuCCAGAu |
| 317 | Scn10a Sp Exon 13_T5 | CACCAuCACCuuGuGCAuCG |
| 318 | Scn10a Sp Exon 13_T6 | ACACuAuAAuGCAGAACuCG |
| 319 | Scn10a Sp Exon 13_T8 | CACCACGAuGCACAAGGuGA |
| 320 | Scn10a Sp Exon 13_T9 | CGuGGuGAACACCAuCuuCA |
| 321 | Scn10a Sp Exon 13_T10 | GGAGCAuGGCuuCGAAGGuA |
| 322 | Scn10a Sp Exon 13_T11 | uAuCuGGAGCAuGGCuuCGA |
| 323 | Scn10a Sp Exon 13_T12 | uGGAGCAuGGCuuCGAAGGu |
| 324 | Scn10a Sp Exon 13_T13 | CGAAGGuAGGGCuCAuGCCA |
| 325 | Scn10a Sp Exon 13_T14 | GGuGuuCACCACGAuGCACA |
| 326 | Scn10a Sp Exon 13_T15 | ACAAGCuGGuCAAGCAGGGu |
| 327 | Scn10a Sp Exon 13_T16 | GAuGuuGCCuAuCuGGAGCA |
| 328 | Scn10a Sp Exon 13_T17 | uuCAuGGCCAuGGAGCACCA |
| 329 | Scn10a Sp Exon 13_T18 | uCuuGAGCuuCACCCACAuG |
| 330 | Scn10a Sp Exon 13_T19 | CuGGGAuuGCuGCCCCAuGu |
| 331 | Scn10a Sp Exon 13_T20 | uGuCuuGAGCuuCACCCACA |
| 332 | Scn10a Sp Exon 13_T21 | GuGAuGGuGAGCuCuGCAAA |
| 333 | Scn10a Sp Exon 13_T22 | GGuGAuGGuGAGCuCuGCAA |
| 334 | Scn10a Sp Exon 14_T1 | uGuGCuGCGGAGCuuCCGCu |
| 335 | Scn10a Sp Exon 14_T2 | GAAAuAAuAGuAuGGGuCGA |
| 23 | Scn10a Sp Exon 14_T3 | GGAAGCuCCGCAGCACAGAC |
| 336 | Scn10a Sp Exon 14_T4 | ACuGuGAGuCuGCuAGAGCu |
| 337 | Scn10a Sp Exon 14_T5 | CACuGuGAGuCuGCuAGAGC |

TABLE 4
Names and sequences of SaCas9 guide RNAs
targeted to the SCN10A gene (Nav1.8)
Nav1.8 SaCas9 gRNAs

| SEQ | | |
|---|---|---|
| ID | | |
| NO: | gRNA Name | spacer sequence (5′-3′) |
| 338 | Scn10a Sa Exon 1_T1 | GCGACGGAAGuuGuuAGuuuCG |
| 38 | Scn10a Sa Exon 1_T3 | AACAACuuCCGuCGCuuuACuC |
| 339 | Scn10a Sa Exon 1_T4 | GuuAGuuuCGAGGGAuCCAAuG |
| 340 | Scn10a Sa Exon 1_T5 | CuuACCCGGuGuGuGCuGuAGA |
| 341 | Scn10a Sa Exon 1_T6 | AGAACuGAuCGGGGAGCCCCuG |
| 32 | Scn10a Sa Exon 1_T7 | uAGAuCCGuuCuACAGCACACA |
| 342 | Scn10a Sa Exon 1_T8 | uCuCuAuCuCCACCAGuGACuC |
| 343 | Scn10a Sa Exon 1_T9 | AAuGAGAAGAuGGAAuuCCCCA |
| 344 | Scn10a Sa Exon 2_T1 | AAGGACuGAAuAGCCACAGGGC |
| 345 | Scn10a Sa Exon 2_T2 | uCuuCuGAuCAGGuuGAAAGGA |
| 346 | Scn10a Sa Exon 3_T1 | CuGACCuuCCAGAGAAAAuuGA |
| 347 | Scn10a Sa Exon 3_T2 | CAAuuuuCuCuGGAAGGuCAGu |
| 348 | Scn10a Sa Exon 3_T3 | CGAACuGACCuuCCAGAGAAAA |
| 349 | Scn10a Sa Exon 4_T2 | CuGGCAAGAGGAuuuuGuCuAA |
| 350 | Scn10a Sa Exon 4_T3 | ACGCuAAAAuCCAGCCAGuuCC |
| 351 | Scn10a Sa Exon 4_T4 | CCuGAGAGAuCCuuGGAACuGG |
| 37 | Scn10a Sa Exon 4_T5 | AuuuuAGCGuCAuuACCCuGGC |
| 352 | Scn10a Sa Exon 4_T7 | GCCuuGAuAAAGAuACuGGCAA |
| 353 | Scn10a Sa Exon 5_T1 | uuGGCACAGCAAuAGAuCuCCG |
| 354 | Scn10a Sa Exon 5_T2 | GGAuCuCAGGCCuGCGGACAuu |
| 355 | Scn10a Sa Exon 5_T4 | uGuuuuuAAuGCuCuAAGAACu |
| 356 | Scn10a Sa Exon 6_T1 | CuCCACuCACGuuuuCuGuGAG |
| 357 | Scn10a Sa Exon 6_T3 | AAACACuuAGGCAGAAGAuGGu |
| 358 | Scn10a Sa Exon 6_T4 | CAuCAGCCAGuuuCuuCACuGA |
| 359 | Scn10a Sa Exon 6_T5 | AACuACuCAuCuCACAGAAAAC |
| 360 | Scn10a Sa Exon 6_T6 | GuCACAuCAGCCAGuuuCuuCA |
| 361 | Scn10a Sa Exon 6_T7 | CAACCuCAAAAAuAAAuGuGuC |
| 362 | Scn10a Sa Exon 6_T8 | uuuAuuuuuGAGGuuGCCCuuG |
| 363 | Scn10a Sa Exon 7_T2 | uCAGAuCCAuuGCCACACAGuA |
| 364 | Scn10a Sa Exon 7_T3 | CuGuGuGGCAAuGGAuCuGACu |
| 365 | Scn10a Sa Exon 7_T4 | GuGGCAAuGGAuCuGACuCAGG |
| 366 | Scn10a Sa Exon 7_T5 | uCuGACCCCuuACuGuGuGGCA |
| 367 | Scn10a Sa Exon 8_T1 | ACCuGCuGGuAGAGGCGuuCCC |
| 368 | Scn10a Sa Exon 8_T2 | CuCACuGuuCCGCCuCAuGACA |
| 369 | Scn10a Sa Exon 8_T3 | CuGCCuuAAAACuuCuGACAAC |
| 370 | Scn10a Sa Exon 8_T4 | uCAAAGCuGGuGuAGuuAAAAu |
| 33 | Scn10a Sa Exon 8_T5 | AGuGAGAGGAAAGCCCAAGCAA |
| 371 | Scn10a Sa Exon 9_T1 | uuuuuuGuGCuCGuAAuCuuCC |
| 372 | Scn10a Sa Exon 9_T3 | uAuAGAuuuuCCCAGAAGuCCu |
| 373 | Scn10a Sa Exon 10_T1 | GCuCuGAuCCuuACAACCAGCG |
| 374 | Scn10a Sa Exon 10_T2 | CuuCGCAGGuGCuAGCAGCACu |
| 375 | Scn10a Sa Exon 10_T3 | CuuACCAuCCuGCGCuGGuuGu |
| 376 | Scn10a Sa Exon 10_T4 | GCGCuGGuuGuAAGGAuCAGAG |
| 377 | Scn10a Sa Exon 10_T5 | ACAACCuCuCuCCACuCCCACA |
| 378 | Scn10a Sa Exon 10_T6 | GAGGuuAAAGGuGAuCCAuuGu |
| 379 | Scn10a Sa Exon 10_T7 | AGAGAAGGCAuAGAAuAAAGCC |
| 380 | Scn10a Sa Exon 10_T8 | AAAAuGCCAGuGAGAGAAGGCA |
| 31 | Scn10a Sa Exon 11_T1 | CCCuGGAGCuGuCGAuGuCuCG |
| 39 | Scn10a Sa Exon 11_T2 | GCCGAGAuAuCuCACuCCCuGA |
| 381 | Scn10a Sa Exon 11_T3 | AAGCCAuCGGGGCuCuCuGCuG |
| 382 | Scn10a Sa Exon 11_T4 | uCCAuCAuCuGuGACuCCCuCA |
| 40 | Scn10a Sa Exon 11_T5 | uGGuGuuCAuCuuCuCCAuGCC |
| 383 | Scn10a Sa Exon 11_T7 | uCGGGGCuCuCuGCuGCuGGGu |
| 384 | Scn10a Sa Exon 11_T8 | uCCAuGCCuGGAGuCAGGGuuG |
| 385 | Scn10a Sa Exon 11_T9 | uCCCuGAGGGAGuCACAGAuGA |
| 386 | Scn10a Sa Exon 11_T10 | uCAuCuuCuCCAuGCCuGGAGu |
| 387 | Scn10a Sa Exon 12_T1 | CAGuAuCAuAACCuCCGuCCuu |
| 34 | Scn10a Sa Exon 12_T2 | ACCuuuCCGGGCCCAAAGGGCA |
| 388 | Scn10a Sa Exon 12_T4 | GAAAGuCuuCuuuuGuCCuGCA |
| 389 | Scn10a Sa Exon 12_T5 | CAAAAGAAGACuuuCuuGuCAG |
| 390 | Scn10a Sa Exon 13_T1 | CCACACuAuAAuGCAGAACuCG |
| 391 | Scn10a Sa Exon 13_T2 | CAuGCuCCAGAuAGGCAACAuC |
| 392 | Scn10a Sa Exon 13_T3 | AGGGAuCCGuCACAAGCCCAAA |
| 393 | Scn10a Sa Exon 13_T4 | uGAuCuGGGAuuGCuGCCCCAu |
| 394 | Scn10a Sa Exon 13_T5 | CuuGuCuCAGAAGuAuCuGAuC |
| 395 | Scn10a Sa Exon 13_T6 | GACAAuuCuCuuuGGGCuuGuG |
| 396 | Scn10a Sa Exon 13_T7 | CuuCuGAGACAAGCuGGuCAAG |
| 397 | Scn10a Sa Exon 13_T8 | AAGGuGAuGGuGAGCuCuGCAA |
| 398 | Scn10a Sa Exon 13_T9 | uGGGCACuuCuGuuCAGACuCC |
| 35 | Scn10a Sa Exon 14_T1 | CuuuGACuGCAuCAuCGuCACu |
| 36 | Scn10a Sa Exon 14_T2 | CACuuCuuCuGGAAAuAAuAGu |
| 399 | Scn10a Sa Exon 14_T3 | GuAAAAAAuAuGGuAAAGACCu |
| 400 | Scn10a Sa Exon 14_T4 | AuACuAuuAuuuCCAGAAGAAG |

TABLE 5
Names, sequences and targeted exons of primers used for sequencing
analysis of CRISPR-mediated editing of the SCN9A gene (Nav1.7)
Nav1.7 NGS Primers

| SEQ | | | |
|---|---|---|---|
| ID | | | |
| NO: | Primer | Sequence | Exon |
| 401 | Nav1-7-Exon-2-1-NGS-F1 | TCGTCGGCAGCGTCAGATGTGTATAAGAGACAGCA | 2-1 |
| | | TCCAGGCCTCTTATGTGAGGAG | |
| 402 | Nav1-7-Exon-2-1-NGS-R1 | GTCTCGTGGGCTCGGAGATGTGTATAAGAGACAGA | |
| | | TAGATGAAGGGCAGCTGTTTGC | |
| 403 | Nav1-7-Exon-2-2-NGS-F1 | TCGTCGGCAGCGTCAGATGTGTATAAGAGACAGTG | 2-2 |
| | | AACAACGCATTGCTGAAAGA | |
| 404 | Nav1-7-Exon-2-2-NGS-R1 | GTCTCGTGGGCTCGGAGATGTGTATAAGAGACAGT | |
| | | TTTATACAGAAGGAAGCCAACAG | |
| 405 | Nav1-7-Exon-3-NGS-F2 | TCGTCGGCAGCGTCAGATGTGTATAAGAGACAGA | 3 |
| | | ACTGCTGATATTGATGTGAAAAA | |
| 406 | Nav1-7-Exon-3-NGS-R2 | GTCTCGTGGGCTCGGAGATGTGTATAAGAGACAGA | |
| | | AAATAGCAAAAATTACACCATAAAGT | |
| 407 | Nav1-7-Exon-4-NGS-F2 | TCGTCGGCAGCGTCAGATGTGTATAAGAGACAGTC | 4 |
| | | CTCAAATATTTCAAATTCCCACTGT | |
| 408 | Nav1-7-Exon-4-NGS-R2 | GTCTCGTGGGCTCGGAGATGTGTATAAGAGACAGT | |
| | | CCCCATCTTCATAAATGCAGTAAC | |
| 409 | Nav1-7-Exon-5-NGS-F1 | TCGTCGGCAGCGTCAGATGTGTATAAGAGACAGA | 5 |
| | | AAGATTTACATGGTGGTTGTATTCTT | |
| 410 | Nav1-7-Exon-5-NGS-R1 | GTCTCGTGGGCTCGGAGATGTGTATAAGAGACAGC | |
| | | TGTGCTGCCTGAGATTTTCAT | |
| 411 | Nav1-7-Exon-6-NGS-F2 | TCGTCGGCAGCGTCAGATGTGTATAAGAGACAGA | 6 |
| | | GCCCCAAACGTAGAAAATACCT | |
| 412 | Nav1-7-Exon-6-NGS-R2 | GTCTCGTGGGCTCGGAGATGTGTATAAGAGACAGC | |
| | | TCCCAAATAGTTGGAGTTATGAGT | |
| 413 | Nav1-7-Exon-7-1-NGS-F2 | TCGTCGGCAGCGTCAGATGTGTATAAGAGACAGGT | 7-1 |
| | | TTCGATTCAGAGGCTTTATGTC | |
| 414 | Nav1-7-Exon-7-1-NGS-R2 | GTCTCGTGGGCTCGGAGATGTGTATAAGAGACAGC | |
| | | TCTCTAGGGTATTCATTATGCTTTCT | |
| 415 | Nav1-7-Exon-7-2-NGS-F2 | TCGTCGGCAGCGTCAGATGTGTATAAGAGACAGG | 7-2 |
| | | AAGCTTTCTGATGTCATGATCC | |
| 416 | Nav1-7-Exon-7-2-NGS-R2 | GTCTCGTGGGCTCGGAGATGTGTATAAGAGACAGG | |
| | | CAACATTTCATTATTAAAAGAGAGCA | |
| 417 | Nav1-7-Exon-8-NGS-F1 | TCGTCGGCAGCGTCAGATGTGTATAAGAGACAGG | 8 |
| | | GACCAGGCCTGAATTTGTAG | |
| 418 | Nav1-7-Exon-8-NGS-R1 | GTCTCGTGGGCTCGGAGATGTGTATAAGAGACAGT | |
| | | TTGCAAACTGACTGAACATTCT | |
| 419 | Nav1-7-Exon-9-NGS-F3 | TCGTCGGCAGCGTCAGATGTGTATAAGAGACAGGT | 9 |
| | | CCTGAAGACACTCTCACCT | |
| 420 | Nav1-7-Exon-9-NGS-R3 | GTCTCGTGGGCTCGGAGATGTGTATAAGAGACAGC | |
| | | AGGCTCTTAACATACACCAGG | |
| 421 | Nav1-7-Exon-10-1-NGS-F1 | TCGTCGGCAGCGTCAGATGTGTATAAGAGACAGTG | 10-1 |
| | | CTCATGCCTGTCAAATTGAAATA | |
| 422 | Nav1-7-Exon-10-1-NGS-R1 | GTCTCGTGGGCTCGGAGATGTGTATAAGAGACAGA | |
| | | CATCTGTTGAAATTCTAATTCTTTCTGT | |
| 423 | Nav1-7-Exon-10-2-NGS-F4 | TCGTCGGCAGCGTCAGATGTGTATAAGAGACAGCT | 10-2 |
| | | AGACGCTGCGTGCTGCT | |
| 424 | Nav1-7-Exon-10-2-NGS-R4 | GTCTCGTGGGCTCGGAGATGTGTATAAGAGACAGA | |
| | | AGGCCAAGCATATACCGCAGA | |
| 425 | Nav1-7-Exon-11-1-NGS-F3 | TCGTCGGCAGCGTCAGATGTGTATAAGAGACAGAT | 11-1 |
| | | GTCCTGTCCTAGGGTTTCCT | |
| 426 | Nav1-7-Exon-11-1-NGS-R3 | GTCTCGTGGGCTCGGAGATGTGTATAAGAGACAGT | |
| | | CAGCATCTCCCTTTTCCTCTC | |
| 427 | Nav1-7-Exon-11-2-NGS-F1 | TCGTCGGCAGCGTCAGATGTGTATAAGAGACAGG | 11-2 |
| | | GCAGCGGCTGAATATACAAGT | |
| 428 | Nav1-7-Exon-11-2-NGS-R1 | GTCTCGTGGGCTCGGAGATGTGTATAAGAGACAGC | |
| | | TCGCCTATGCCCTTCGAC | |
| 429 | Nav1-7-Exonl 1-3-NGS-F3 | TCGTCGGCAGCGTCAGATGTGTATAAGAGACAGA | 11-3 |
| | | GAATCAAAAGAAGCTCTCCAGTG | |
| 430 | Nav1-7-Exonl 1-3-NGS-R3 | GTCTCGTGGGCTCGGAGATGTGTATAAGAGACAGT | |
| | | CACTCACTATCCTCTCCCGA | |
| 431 | Nav1-7-Exon12-1-NGS-F6 | TCGTCGGCAGCGTCAGATGTGTATAAGAGACAGA | 12-1 |
| | | GTGTACTTCTATCAGTAGGTGCTT | |
| 432 | Nav1-7-Exon12-1-NGS-R6 | GTCTCGTGGGCTCGGAGATGTGTATAAGAGACAGG | |
| | | ACCTACTGGCTTGGCTGAT | |
| 433 | Nav1-7-Exon-12-2-NGS-F2 | TCGTCGGCAGCGTCAGATGTGTATAAGAGACAGA | 12-2 |
| | | AGCAGCAGAACAAGTCTTTTTAGTT | |
| 434 | Nav1-7-Exon-12-2-NGS-R2 | GTCTCGTGGGCTCGGAGATGTGTATAAGAGACAGA | |
| | | CCAGGGAGACCACACCGT | |
| 435 | Nav1-7-Exon12-3-NGS-F6 | TCGTCGGCAGCGTCAGATGTGTATAAGAGACAGAC | 12-3 |
| | | AGCATTTTTGGAGACAATGAGA | |
| 436 | Nav1-7-Exon12-3-NGS-R6 | GTCTCGTGGGCTCGGAGATGTGTATAAGAGACAGA | |
| | | TGCCTGAGCTATGTAAAACGTC | |
| 437 | Nav1-7-Exon-13-1-NGS-F1 | TCGTCGGCAGCGTCAGATGTGTATAAGAGACAGCC | 13-1 |
| | | CAGCAATCTAGGCTCTACT | |
| 438 | Nav1-7-Exon-13-1-NGS-R1 | GTCTCGTGGGCTCGGAGATGTGTATAAGAGACAGC | |
| | | CTTCCACAGTGTTTGTTAATATGC | |
| 439 | Nav1-7-Exon-13-2-NGS-F4 | TCGTCGGCAGCGTCAGATGTGTATAAGAGACAGTC | 13-2 |
| | | AAATACACAAGAAAAGGCGTTG | |
| 440 | Nav1-7-Exon-13-2-NGS-R4 | GTCTCGTGGGCTCGGAGATGTGTATAAGAGACAGA | |
| | | CAATTCCATCAGTATCCATTGGT | |
| 441 | Nav1-7-Exon-14-1-NGS-F1 | TCGTCGGCAGCGTCAGATGTGTATAAGAGACAGG | 14-1 |
| | | GTTAGGAGTGAAACAGACAAATGG | |
| 442 | Nav1-7-Exon-14-1-NGS-R1 | GTCTCGTGGGCTCGGAGATGTGTATAAGAGACAGT | |
| | | GGTGTTCCATAGCCATAAATAATGTG | |
| 443 | Nav1-7-Exon-14-2-NGS-F2 | TCGTCGGCAGCGTCAGATGTGTATAAGAGACAGTC | 14-2 |
| | | TTGATCTGGAATTGCTCTCCAT | |
| 444 | Nav1-7-Exon-14-2-NGS-R2 | GTCTCGTGGGCTCGGAGATGTGTATAAGAGACAGA | |
| | | CAATGATGACAACTAAAAAGAGAAACT | |
| 445 | Nav1-7-Exon-15-1-NGS-F2 | TCGTCGGCAGCGTCAGATGTGTATAAGAGACAGAT | 15-1 |
| | | CATTGTGTTGATTTCCTGTTTTCT | |
| 446 | Nav1-7-Exon-15-1-NGS-R2 | GTCTCGTGGGCTCGGAGATGTGTATAAGAGACAGC | |
| | | CAGTCTGAATGATCGCAGAAC | |
| 447 | Nav1-7-Exon-15-2-NGS-F2 | TCGTCGGCAGCGTCAGATGTGTATAAGAGACAGTG | 15-2 |
| | | CAGCTGAAATGGTATTAAAACTGA | |
| 448 | Nav1-7-Exon-15-2-NGS-R2 | GTCTCGTGGGCTCGGAGATGTGTATAAGAGACAGT | |
| | | GCAAAACCAAAGAAATACCCCTT | |

TABLE 6
Names, sequences and targeted exons of primers used for sequencing
analysis of CRISPR-mediated editing of the SCN10A gene (Nav1.8)
Nav1.8 NGS Primers

| SEQ | | | |
|---|---|---|---|
| ID NO: | Primer | Sequence | Exon |
| 449 | Nav1-8-Exon-1-1-NGS-F4 | TCGTCGGCAGCGTCAGATGTGTATAAGAGACAGGC | 1-1 |
| | | TGTCACCTCTCTGTGGTT | |
| 450 | Nav1-8-Exon-1-1-NGS-R4 | GTCTCGTGGGCTCGGAGATGTGTATAAGAGACAGT | |
| | | AGAACTTGGGCAGCTGGTT | |
| 451 | Nav1-8-Exon-1-2-NGS-F1 | TCGTCGGCAGCGTCAGATGTGTATAAGAGACAGCC | 1-2 |
| | | AAGCAGGGAACAAAGAAA | |
| 452 | Nav1-8-Exon-1-2-NGS-R1 | GTCTCGTGGGCTCGGAGATGTGTATAAGAGACAGC | |
| | | CGAGACTTCCTCTCCAAGA | |
| 453 | Nav1-8-Exon-2-1-NGS-F3 | TCGTCGGCAGCGTCAGATGTGTATAAGAGACAGGC | 2 |
| | | CTGAGATAATGCCTCTCATGT | |
| 454 | Nav1-8-Exon-2-1-NGS-R3 | GTCTCGTGGGCTCGGAGATGTGTATAAGAGACAGA | |
| | | CTGGACACAGTAGGCAAGG | |
| 455 | Nav1-8-Exon-3-1-NGS-F1 | TCGTCGGCAGCGTCAGATGTGTATAAGAGACAGTG | 3 |
| | | TAATCATTCAGCATCAAGGTG | |
| 456 | Nav1-8-Exon-3-1-NGS-R1 | GTCTCGTGGGCTCGGAGATGTGTATAAGAGACAGA | |
| | | CAAAGACTGCCAAGTGAAGGA | |
| 457 | Nav1-8-Exon-4-1-NGS-F3 | TCGTCGGCAGCGTCAGATGTGTATAAGAGACAGCA | 4 |
| | | TCCCTCCCTCCTGGAAGA | |
| 458 | Nav1-8-Exon-4-1-NGS-R3 | GTCTCGTGGGCTCGGAGATGTGTATAAGAGACAGC | |
| | | CCCTCTCCTATCACACATGC | |
| 459 | Nav1-8-Exon-5-1-NGS-F3 | TCGTCGGCAGCGTCAGATGTGTATAAGAGACAGA | 5 |
| | | GGCTAATGATACCCCAGGT | |
| 460 | Nav1-8-Exon-5-1-NGS-R3 | GTCTCGTGGGCTCGGAGATGTGTATAAGAGACAGA | |
| | | GTCTTTGCCCTGGAACCTT | |
| 461 | Nav1-8-Exon-6-1-NGS-F4 | TCGTCGGCAGCGTCAGATGTGTATAAGAGACAGGT | 6-1 |
| | | GTGCTCCGTGTGTGCTAC | |
| 462 | Nav1-8-Exon-6-1-NGS-R4 | GTCTCGTGGGCTCGGAGATGTGTATAAGAGACAGA | |
| | | ACCAGACCTTGGTCCCTATG | |
| 463 | Nav1-8-Exon-6-2-NGS-F4 | TCGTCGGCAGCGTCAGATGTGTATAAGAGACAGCT | 6-2 |
| | | TCAAGGGCAACCTCAAAA | |
| 464 | Nav1-8-Exon-6-2-NGS-R4 | GTCTCGTGGGCTCGGAGATGTGTATAAGAGACAGA | |
| | | TTTCCTTGCAAGAGGGATG | |
| 465 | Nav1-8-Exon-7-1-NGS-F1 | TCGTCGGCAGCGTCAGATGTGTATAAGAGACAGAT | 7 |
| | | TGCATTCACCACACAAGG | |
| 466 | Nav1-8-Exon-7-1-NGS-R1 | GTCTCGTGGGCTCGGAGATGTGTATAAGAGACAGA | |
| | | TGCCAAGGACAAGATGGAG | |
| 467 | Nav1-8-Exon-8-1-NGS-F1 | TCGTCGGCAGCGTCAGATGTGTATAAGAGACAGTG | 8 |
| | | GGCTACCTTGTCTGCAAT | |
| 468 | Nav1-8-Exon-8-1-NGS-R1 | GTCTCGTGGGCTCGGAGATGTGTATAAGAGACAGA | |
| | | GCCTCCAACCAAGTCTGC | |
| 469 | Nav1-8-Exon-9-1-NGS-F2 | TCGTCGGCAGCGTCAGATGTGTATAAGAGACAGCC | 9-1 |
| | | TGGAGGAGGCTGACTTAAA | |
| 470 | Nav1-8-Exon-9-1-NGS-R2 | GTCTCGTGGGCTCGGAGATGTGTATAAGAGACAGA | |
| | | GGGCCTCCTGGAACTTCTT | |
| 471 | Nav1-8-Exon-9-2-NGS-F4 | TCGTCGGCAGCGTCAGATGTGTATAAGAGACAGTG | 9-2 |
| | | CAGACCCTGAGGACTTCT | |
| 472 | Nav1-8-Exon-9-2-NGS-R4 | GTCTCGTGGGCTCGGAGATGTGTATAAGAGACAGT | |
| | | GGACAGTCTGCAACCTTCT | |
| 473 | Nav1-8-Exon-10-1-NGS-F5 | TCGTCGGCAGCGTCAGATGTGTATAAGAGACAGTC | 10 |
| | | ATGCTAAGTCCAAGCAAATACT | |
| 474 | Nav1-8-Exon-10-1-NGS-R5 | GTCTCGTGGGCTCGGAGATGTGTATAAGAGACAGG | |
| | | CAGCTGCAATGGTGGGTAA | |
| 475 | Nav1-8-Exon-11-1-NGS-F6 | TCGTCGGCAGCGTCAGATGTGTATAAGAGACAGTT | 11-1 |
| | | CCAGCCTTCTTGCTCCTTT | |
| 476 | Nav1-8-Exon-11-1-NGS-R6 | GTCTCGTGGGCTCGGAGATGTGTATAAGAGACAGA | |
| | | GTCAGGGTTGCTGGGTTGA | |
| 477 | Nav1-8-Exon-11-2-NGS-F3 | TCGTCGGCAGCGTCAGATGTGTATAAGAGACAGCC | 11-2 |
| | | CTGAGGGAGTCACAGATG | |
| 478 | Nav1-8-Exon-11-2-NGS-R3 | GTCTCGTGGGCTCGGAGATGTGTATAAGAGACAGG | |
| | | CTCAAGGCTTCTAGGTGGA | |
| 479 | Nav1-8-Exon-12-1-NGS-F2 | TCGTCGGCAGCGTCAGATGTGTATAAGAGACAGTT | 12 |
| | | TGCCATGAAGATGTCAGG | |
| 480 | Nav1-8-Exon-12-1-NGS-R2 | GTCTCGTGGGCTCGGAGATGTGTATAAGAGACAGT | |
| | | GCTCACATGGGAATTCATC | |
| 481 | Nav1-8-Exon-13-1-NGS-F2 | TCGTCGGCAGCGTCAGATGTGTATAAGAGACAGA | 13-1 |
| | | GAGGATGACCGCAGAATTG | |
| 482 | Nav1-8-Exon-13-1-NGS-R2 | GTCTCGTGGGCTCGGAGATGTGTATAAGAGACAGA | |
| | | TGCACAAGGTGATGGTGAG | |
| 483 | Nav1-8-Exon-13-2-NGS-F4 | TCGTCGGCAGCGTCAGATGTGTATAAGAGACAGCT | 13-2 |
| | | TGACCAGCTTGTCTCAGAAG | |
| 484 | Nav1-8-Exon-13-2-NGS-R4 | GTCTCGTGGGCTCGGAGATGTGTATAAGAGACAGA | |
| | | CTGCACCCTGCCATCAT | |
| 485 | Nav1-8-Exon-14-1-NGS-F1 | TCGTCGGCAGCGTCAGATGTGTATAAGAGACAGAC | 14-1 |
| | | CCCACAGATCCCACTGT | |
| 486 | Nav1-8-Exon-14-1-NGS-R1 | GTCTCGTGGGCTCGGAGATGTGTATAAGAGACAGC | |
| | | ACAGACAGGCTTCCCTTCTT | |
| 487 | Nav1-8-Exon-14-2-NGS-F1 | TCGTCGGCAGCGTCAGATGTGTATAAGAGACAGTG | 14-2 |
| | | CTGAAATGGTCTTCAAAATC | |
| 488 | Nav1-8-Exon-14-2-NGS-R1 | GTCTCGTGGGCTCGGAGATGTGTATAAGAGACAGT | |
| | | GAATCTGGGTGGGAGTTTC | |

TABLE 7
Total list of 1,471 putative off-target sites predicted across the 40 guides

| chr | start | end | chr | start | end | chr | start | end |
|---|---|---|---|---|---|---|---|---|
| chr1 | 101673862 | 101673883 | chr6 | 81389456 | 81389478 | chr8 | 18547775 | 18547797 |
| chr1 | 181263539 | 181263560 | chr7 | 116885434 | 116885456 | chr9 | 121398998 | 121399019 |
| chr1 | 230060685 | 230060706 | chr8 | 101163760 | 101163782 | chr9 | 90536880 | 90536903 |
| chr1 | 57212888 | 57212911 | chr8 | 102040037 | 102040058 | chrX | 154575630 | 154575653 |
| chr10 | 129962874 | 129962895 | chr8 | 32017421 | 32017443 | chrX | 154628777 | 154628800 |
| chr10 | 84935685 | 84935706 | chr9 | 134428917 | 134428939 | chr1 | 218298983 | 218299004 |
| chr10 | 8530131 | 8530152 | chr9 | 41739585 | 41739607 | chr1 | 42210368 | 42210390 |
| chr11 | 11657568 | 11657590 | chr9 | 61575358 | 61575380 | chr1 | 58885286 | 58885307 |
| chr11 | 118434780 | 118434801 | chr9 | 67636882 | 67636904 | chr1 | 99737182 | 99737204 |
| chr11 | 19769579 | 19769600 | chrX | 103932345 | 103932367 | chr10 | 119436660 | 119436681 |
| chr11 | 44591707 | 44591728 | chrX | 149687593 | 149687615 | chr10 | 78303428 | 78303451 |
| chr11 | 60420702 | 60420725 | chrX | 77969229 | 77969250 | chr11 | 105632443 | 105632465 |
| chr11 | 79356353 | 79356374 | chrY | 17093954 | 17093976 | chr11 | 49938853 | 49938876 |
| chr11 | 81605512 | 81605533 | chr1 | 106130660 | 106130681 | chr12 | 103863493 | 103863514 |
| chr12 | 118776061 | 118776082 | chr1 | 17346264 | 17346285 | chr12 | 92649761 | 92649783 |
| chr12 | 54000745 | 54000768 | chr1 | 191298075 | 191298097 | chr13 | 112185105 | 112185127 |
| chr12 | 95630598 | 95630619 | chr1 | 236890187 | 236890208 | chr13 | 29354998 | 29355019 |
| chr13 | 110180589 | 110180611 | chr1 | 244884251 | 244884272 | chr14 | 56541130 | 56541151 |
| chr13 | 40148712 | 40148733 | chr10 | 79776620 | 79776641 | chr14 | 63594134 | 63594155 |
| chr13 | 60239631 | 60239653 | chr10 | 87298770 | 87298791 | chr15 | 79057189 | 79057210 |
| chr13 | 66558377 | 66558400 | chr10 | 87439176 | 87439197 | chr16 | 1024374 | 1024397 |
| chr14 | 104081758 | 104081780 | chr11 | 133566119 | 133566141 | chr17 | 57649035 | 57649057 |
| chr14 | 104550954 | 104550976 | chr11 | 18269330 | 18269351 | chr2 | 142368179 | 142368200 |
| chr14 | 70672101 | 70672122 | chr11 | 31057715 | 31057736 | chr2 | 166286371 | 166286393 |
| chr14 | 72884578 | 72884599 | chr11 | 86679889 | 86679910 | chr20 | 24803468 | 24803489 |
| chr14 | 76371571 | 76371593 | chr13 | 26095101 | 26095122 | chr20 | 24803468 | 24803490 |
| chr15 | 75765848 | 75765871 | chr13 | 88051002 | 88051025 | chr20 | 25276655 | 25276676 |
| chr16 | 51266796 | 51266817 | chr15 | 54619157 | 54619179 | chr20 | 57304528 | 57304551 |
| chr16 | 66970656 | 66970677 | chr16 | 78175674 | 78175695 | chr20 | 63700780 | 63700801 |
| chr17 | 38736344 | 38736365 | chr16 | 85698295 | 85698317 | chr3 | 12181396 | 12181417 |
| chr17 | 5662660 | 5662682 | chr18 | 4435202 | 4435223 | chr3 | 127363721 | 127363742 |
| chr17 | 77914635 | 77914656 | chr18 | 63953514 | 63953535 | chr3 | 73435663 | 73435686 |
| chr19 | 14021634 | 14021656 | chr18 | 75626393 | 75626414 | chr4 | 148797789 | 148797810 |
| chr2 | 127092215 | 127092238 | chr19 | 16143760 | 16143782 | chr4 | 98007120 | 98007141 |
| chr2 | 134821956 | 134821977 | chr19 | 3636896 | 3636918 | chr5 | 141787884 | 141787905 |
| chr2 | 178940831 | 178940853 | chr2 | 102073639 | 102073660 | chr5 | 155635156 | 155635177 |
| chr20 | 20432289 | 20432311 | chr2 | 111189580 | 111189601 | chr6 | 104164934 | 104164956 |
| chr20 | 58601338 | 58601359 | chr2 | 15761798 | 15761820 | chr7 | 139583922 | 139583943 |
| chr21 | 35410460 | 35410482 | chr2 | 178287459 | 178287480 | chr7 | 142193746 | 142193769 |
| chr22 | 39957473 | 39957496 | chr2 | 81854796 | 81854819 | chr7 | 47045289 | 47045310 |
| chr3 | 119381654 | 119381676 | chr2 | 96711649 | 96711670 | chr7 | 99403691 | 99403712 |
| chr3 | 140431388 | 140431409 | chr20 | 46241748 | 46241769 | chr9 | 89919212 | 89919233 |
| chr3 | 38755787 | 38755809 | chr20 | 58037844 | 58037865 | chr9 | 97393314 | 97393335 |
| chr4 | 109874522 | 109874543 | chr20 | 61210620 | 61210641 | chrX | 101837971 | 101837993 |
| chr4 | 135986289 | 135986310 | chr21 | 31457963 | 31457984 | chrX | 102321859 | 102321881 |
| chr4 | 162888411 | 162888432 | chr3 | 38771311 | 38771333 | chrX | 102365234 | 102365256 |
| chr4 | 95257196 | 95257217 | chr3 | 67894307 | 67894329 | chrX | 42045623 | 42045644 |
| chr5 | 91935098 | 91935119 | chr5 | 150570292 | 150570313 | chrX | 95359671 | 95359693 |
| chr6 | 106430770 | 106430791 | chr5 | 163518469 | 163518491 | chr1 | 106905030 | 106905051 |
| chr6 | 154267869 | 154267892 | chr6 | 41702022 | 41702043 | chr1 | 187369871 | 187369893 |
| chr7 | 14664086 | 14664107 | chr6 | 42338824 | 42338847 | chr1 | 217283942 | 217283964 |
| chr7 | 41078138 | 41078159 | chr8 | 133739469 | 133739491 | chr1 | 67514969 | 67514990 |
| chr7 | 56759248 | 56759270 | chr8 | 141160774 | 141160795 | chr1 | 73958112 | 73958133 |
| chr7 | 63816239 | 63816261 | chr8 | 20158237 | 20158258 | chr10 | 131976154 | 131976175 |
| chr8 | 117647612 | 117647633 | chr8 | 23430667 | 23430688 | chr10 | 4150457 | 4150479 |
| chr8 | 19663295 | 19663316 | chr9 | 130371904 | 130371925 | chr10 | 45658400 | 45658421 |
| chr8 | 26849329 | 26849350 | chr9 | 136746114 | 136746135 | chr10 | 54308353 | 54308374 |
| chr8 | 46389287 | 46389309 | chr9 | 31741140 | 31741162 | chr10 | 86859468 | 86859489 |
| chr8 | 500777 | 500799 | chrX | 38085667 | 38085689 | chr10 | 95305836 | 95305858 |
| chr8 | 67346602 | 67346623 | chrX | 88090240 | 88090261 | chr12 | 16683841 | 16683862 |
| chr9 | 109783158 | 109783179 | chr1 | 104446115 | 104446136 | chr12 | 17604379 | 17604400 |
| chr9 | 130987364 | 130987385 | chr1 | 182332402 | 182332423 | chr12 | 83263173 | 83263195 |
| chr9 | 132448490 | 132448511 | chr1 | 183710217 | 183710238 | chr16 | 3841926 | 3841947 |
| chr9 | 3023108 | 3023129 | chr1 | 208748851 | 208748872 | chr18 | 71712771 | 71712794 |
| chr9 | 70925755 | 70925777 | chr1 | 240969802 | 240969823 | chr2 | 155442093 | 155442116 |
| chr9 | 78073257 | 78073279 | chr1 | 26574711 | 26574733 | chr2 | 166286585 | 166286607 |
| chrX | 130376370 | 130376393 | chr1 | 30456582 | 30456603 | chr2 | 171528653 | 171528674 |
| chrX | 39650052 | 39650074 | chr1 | 30777702 | 30777725 | chr2 | 19830616 | 19830637 |
| chrY | 6906016 | 6906037 | chr1 | 37625208 | 37625231 | chr2 | 206933530 | 206933551 |
| chr1 | 186659904 | 186659926 | chr1 | 51905268 | 51905289 | chr2 | 23224743 | 23224764 |
| chr1 | 247936465 | 247936486 | chr1 | 95744205 | 95744228 | chr21 | 29458589 | 29458610 |
| chr10 | 105539151 | 105539172 | chr10 | 109509276 | 109509299 | chr22 | 29667158 | 29667180 |
| chr10 | 11271522 | 11271544 | chr10 | 113476180 | 113476201 | chr3 | 103315028 | 103315050 |
| chr10 | 120030919 | 120030940 | chr10 | 114453754 | 114453775 | chr3 | 161401860 | 161401882 |
| chr10 | 122199548 | 122199569 | chr10 | 118020250 | 118020271 | chr3 | 31354038 | 31354059 |
| chr10 | 21055795 | 21055816 | chr10 | 35888611 | 35888633 | chr4 | 159960343 | 159960364 |
| chr10 | 49303209 | 49303230 | chr10 | 47483140 | 47483161 | chr5 | 140291535 | 140291557 |
| chr11 | 12054766 | 12054789 | chr10 | 71103889 | 71103910 | chr5 | 154340017 | 154340038 |
| chr11 | 134957078 | 134957099 | chr10 | 8187525 | 8187546 | chr6 | 111811118 | 111811140 |
| chr11 | 72025745 | 72025766 | chr10 | 82320936 | 82320957 | chr6 | 17932393 | 17932416 |
| chr12 | 119372784 | 119372807 | chr10 | 97073249 | 97073270 | chr7 | 24855398 | 24855419 |
| chr12 | 60148276 | 60148297 | chr10 | 99797253 | 99797275 | chr7 | 98797096 | 98797118 |
| chr13 | 42848194 | 42848215 | chr11 | 106591429 | 106591451 | chr8 | 112670565 | 112670588 |
| chr13 | 58386752 | 58386775 | chr11 | 112369728 | 112369751 | chr8 | 30460352 | 30460373 |
| chr13 | 75702660 | 75702682 | chr11 | 11294441 | 11294463 | chr9 | 26330106 | 26330127 |
| chr14 | 24835976 | 24835997 | chr11 | 23994069 | 23994092 | chr9 | 86133463 | 86133485 |
| chr14 | 29623510 | 29623531 | chr11 | 29830201 | 29830222 | chrX | 146222562 | 146222583 |
| chr14 | 46653453 | 46653475 | chr11 | 35420641 | 35420662 | chrX | 17601216 | 17601237 |
| chr14 | 72079604 | 72079627 | chr11 | 61138088 | 61138110 | chrX | 56719178 | 56719201 |
| chr15 | 38307571 | 38307592 | chr11 | 69777241 | 69777264 | chr1 | 161715957 | 161715978 |
| chr16 | 30086751 | 30086772 | chr11 | 72020177 | 72020198 | chr1 | 186721495 | 186721516 |
| chr16 | 59179342 | 59179363 | chr11 | 72658233 | 72658256 | chr1 | 199465695 | 199465717 |
| chr17 | 74203946 | 74203967 | chr11 | 84482547 | 84482568 | chr1 | 234230046 | 234230069 |
| chr18 | 14964141 | 14964162 | chr11 | 96164837 | 96164860 | chr1 | 246597563 | 246597584 |
| chr18 | 70296440 | 70296462 | chr12 | 109406803 | 109406825 | chr1 | 9340847 | 9340868 |
| chr19 | 55892885 | 55892908 | chr13 | 113809849 | 113809870 | chr10 | 101350793 | 101350816 |
| chr2 | 204557623 | 204557646 | chr13 | 26875905 | 26875926 | chr10 | 103066765 | 103066787 |
| chr2 | 213875062 | 213875083 | chr13 | 27416390 | 27416411 | chr10 | 49429090 | 49429111 |
| chr2 | 236094319 | 236094340 | chr13 | 29693044 | 29693067 | chr11 | 12731192 | 12731214 |
| chr2 | 81092829 | 81092850 | chr13 | 42970814 | 42970835 | chr11 | 94300239 | 94300262 |
| chr20 | 39790561 | 39790582 | chr14 | 36801548 | 36801569 | chr12 | 105242950 | 105242971 |
| chr21 | 22714600 | 22714621 | chr14 | 59632197 | 59632218 | chr12 | 128011654 | 128011676 |
| chr21 | 36945184 | 36945207 | chr14 | 91903881 | 91903902 | chr12 | 30430479 | 30430500 |
| chr3 | 38756760 | 38756782 | chr14 | 98960049 | 98960070 | chr12 | 88935275 | 88935296 |
| chr3 | 38909083 | 38909105 | chr14 | 99595608 | 99595630 | chr12 | 89530030 | 89530053 |
| chr3 | 62160422 | 62160443 | chr15 | 100390078 | 100390100 | chr13 | 107193322 | 107193343 |
| chr4 | 11569781 | 11569802 | chr15 | 27968205 | 27968227 | chr13 | 36103807 | 36103830 |
| chr4 | 4596463 | 4596484 | chr15 | 40289231 | 40289252 | chr14 | 36905708 | 36905731 |
| chr4 | 86891508 | 86891529 | chr15 | 72631643 | 72631664 | chr14 | 69696064 | 69696087 |
| chr5 | 125913329 | 125913350 | chr15 | 74105587 | 74105608 | chr14 | 78054812 | 78054835 |
| chr5 | 152687865 | 152687888 | chr16 | 28011042 | 28011063 | chr15 | 23634421 | 23634442 |
| chr5 | 28941639 | 28941660 | chr16 | 28181839 | 28181860 | chr15 | 63926417 | 63926438 |
| chr5 | 50928286 | 50928307 | chr16 | 4699815 | 4699837 | chr15 | 82103361 | 82103382 |
| chr5 | 73347573 | 73347594 | chr16 | 60430147 | 60430169 | chr15 | 94230598 | 94230619 |
| chr7 | 154662667 | 154662688 | chr16 | 83976871 | 83976892 | chr16 | 63947100 | 63947121 |
| chr7 | 154663811 | 154663832 | chr16 | 86429392 | 86429414 | chr17 | 19807664 | 19807685 |
| chr7 | 90972309 | 90972330 | chr17 | 1162115 | 1162136 | chr17 | 3505544 | 3505567 |
| chr7 | 98854385 | 98854406 | chr17 | 30090056 | 30090077 | chr17 | 44071718 | 44071739 |
| chr8 | 41214468 | 41214491 | chr17 | 44854537 | 44854558 | chr17 | 4995262 | 4995283 |
| chr8 | 49406396 | 49406417 | chr17 | 57642792 | 57642813 | chr18 | 25386572 | 25386593 |
| chr8 | 50663214 | 50663235 | chr18 | 23235311 | 23235332 | chr18 | 44666298 | 44666319 |
| chr8 | 51175334 | 51175357 | chr18 | 57527960 | 57527982 | chr18 | 56303935 | 56303956 |
| chr8 | 62677817 | 62677838 | chr18 | 60250771 | 60250793 | chr18 | 58118525 | 58118546 |
| chr9 | 27101925 | 27101948 | chr18 | 79942923 | 79942944 | chr18 | 7237437 | 7237458 |
| chr1 | 116387955 | 116387976 | chr19 | 15670796 | 15670818 | chr19 | 28344658 | 28344679 |
| chr1 | 14173045 | 14173066 | chr19 | 30910106 | 30910127 | chr2 | 11018267 | 11018288 |
| chr1 | 155713414 | 155713435 | chr19 | 45792252 | 45792273 | chr2 | 142234899 | 142234920 |
| chr1 | 170096719 | 170096740 | chr19 | 7898697 | 7898718 | chr2 | 157696547 | 157696570 |
| chr1 | 175876599 | 175876620 | chr2 | 111551550 | 111551571 | chr2 | 15894912 | 15894935 |
| chr1 | 202292624 | 202292647 | chr2 | 120554544 | 120554565 | chr2 | 166286354 | 166286376 |
| chr1 | 210569059 | 210569080 | chr2 | 126691363 | 126691384 | chr2 | 169738897 | 169738918 |
| chr1 | 50667093 | 50667114 | chr2 | 126694948 | 126694969 | chr2 | 174221489 | 174221510 |
| chr1 | 66329779 | 66329800 | chr2 | 180785404 | 180785426 | chr2 | 198625481 | 198625502 |
| chr1 | 9120177 | 9120198 | chr2 | 191197836 | 191197858 | chr2 | 213460469 | 213460491 |
| chr10 | 118339203 | 118339224 | chr2 | 240752376 | 240752397 | chr2 | 216853354 | 216853375 |
| chr10 | 29318701 | 29318722 | chr2 | 40393626 | 40393648 | chr2 | 30384729 | 30384752 |
| chr10 | 30605633 | 30605654 | chr2 | 87310047 | 87310068 | chr2 | 50247269 | 50247290 |
| chr10 | 78251913 | 78251934 | chr20 | 24761423 | 24761445 | chr20 | 17320938 | 17320959 |
| chr10 | 90894658 | 90894681 | chr20 | 48105588 | 48105609 | chr20 | 19474376 | 19474397 |
| chr10 | 9240868 | 9240890 | chr20 | 63206596 | 63206618 | chr20 | 57549695 | 57549717 |
| chr11 | 119661275 | 119661296 | chr21 | 34811774 | 34811795 | chr22 | 45538244 | 45538265 |
| chr11 | 122398618 | 122398639 | chr21 | 37675033 | 37675056 | chr3 | 10761073 | 10761095 |
| chr11 | 128153457 | 128153478 | chr22 | 25084069 | 25084092 | chr3 | 122197839 | 122197860 |
| chr11 | 35876123 | 35876144 | chr22 | 29716915 | 29716936 | chr3 | 124433870 | 124433891 |
| chr12 | 109444959 | 109444980 | chr22 | 37312013 | 37312034 | chr3 | 142893230 | 142893251 |
| chr12 | 114163696 | 114163719 | chr3 | 116664004 | 116664025 | chr3 | 172385878 | 172385899 |
| chr12 | 114794612 | 114794634 | chr3 | 165846142 | 165846163 | chr3 | 70924914 | 70924936 |
| chr12 | 15291996 | 15292018 | chr3 | 185268854 | 185268875 | chr3 | 8723005 | 8723026 |
| chr12 | 20864724 | 20864745 | chr3 | 19220065 | 19220086 | chr3 | 95332559 | 95332580 |
| chr12 | 27323061 | 27323083 | chr3 | 28603456 | 28603477 | chr3 | 98072324 | 98072345 |
| chr12 | 84921525 | 84921546 | chr3 | 38793784 | 38793806 | chr4 | 111062514 | 111062535 |
| chr13 | 100235136 | 100235157 | chr3 | 63674978 | 63675001 | chr4 | 18864625 | 18864647 |
| chr13 | 21762339 | 21762362 | chr4 | 106491924 | 106491946 | chr4 | 32279580 | 32279603 |
| chr13 | 32618495 | 32618517 | chr4 | 117117815 | 117117838 | chr4 | 85659807 | 85659828 |
| chr13 | 33776819 | 33776840 | chr4 | 119616610 | 119616632 | chr5 | 169266180 | 169266203 |
| chr13 | 43998345 | 43998366 | chr4 | 139219960 | 139219983 | chr5 | 179335776 | 179335797 |
| chr13 | 78341045 | 78341066 | chr4 | 185051888 | 185051909 | chr6 | 108638293 | 108638316 |
| chr15 | 25853638 | 25853659 | chr4 | 24022746 | 24022767 | chr6 | 131453651 | 131453672 |
| chr15 | 62481354 | 62481375 | chr4 | 52789300 | 52789321 | chr6 | 35275738 | 35275760 |
| chr15 | 74878475 | 74878498 | chr4 | 82398573 | 82398596 | chr6 | 40784617 | 40784638 |
| chr16 | 6607972 | 6607993 | chr4 | 85087003 | 85087024 | chr6 | 70439658 | 70439680 |
| chr16 | 6716297 | 6716318 | chr5 | 107883322 | 107883343 | chr7 | 103995304 | 103995325 |
| chr17 | 4106740 | 4106761 | chr5 | 111925220 | 111925241 | chr7 | 110511327 | 110511348 |
| chr17 | 43216123 | 43216145 | chr5 | 143222451 | 143222472 | chr7 | 27305711 | 27305732 |
| chr17 | 50328380 | 50328401 | chr5 | 159866928 | 159866949 | chr7 | 30322503 | 30322524 |
| chr17 | 61461790 | 61461811 | chr5 | 32666438 | 32666459 | chr7 | 68363358 | 68363380 |
| chr17 | 81905722 | 81905743 | chr5 | 42894065 | 42894086 | chr7 | 97668279 | 97668302 |
| chr18 | 73085391 | 73085412 | chr5 | 42899359 | 42899380 | chrX | 140493338 | 140493359 |
| chr19 | 29235109 | 29235131 | chr5 | 78720216 | 78720237 | chrX | 15614904 | 15614926 |
| chr19 | 45003779 | 45003802 | chr5 | 8655482 | 8655503 | chrX | 30782422 | 30782443 |
| chr19 | 47511671 | 47511692 | chr5 | 9135657 | 9135678 | chrX | 31984015 | 31984036 |
| chr19 | 55085146 | 55085169 | chr6 | 113222662 | 113222683 | chrX | 86190344 | 86190365 |
| chr2 | 109228516 | 109228539 | chr6 | 115538457 | 115538478 | chrY | 19917588 | 19917609 |
| chr2 | 142539467 | 142539488 | chr6 | 31688661 | 31688684 | chr1 | 178061425 | 178061447 |
| chr2 | 156144910 | 156144931 | chr6 | 43831465 | 43831486 | chr1 | 2402386 | 2402407 |
| chr2 | 191243299 | 191243320 | chr6 | 87701119 | 87701140 | chr1 | 72287683 | 72287705 |
| chr2 | 196103556 | 196103579 | chr7 | 10594397 | 10594418 | chr1 | 76614806 | 76614827 |
| chr2 | 203787061 | 203787083 | chr7 | 12619084 | 12619105 | chr1 | 89901148 | 89901169 |
| chr2 | 214949817 | 214949839 | chr7 | 149003021 | 149003042 | chr10 | 85000194 | 85000215 |
| chr2 | 234666709 | 234666730 | chr7 | 18335788 | 18335809 | chr11 | 108491260 | 108491281 |
| chr2 | 28172483 | 28172506 | chr7 | 24705842 | 24705863 | chr11 | 66423974 | 66423995 |
| chr2 | 3057588 | 3057611 | chr7 | 286530 | 286551 | chr12 | 71595067 | 71595090 |
| chr2 | 36232821 | 36232842 | chr7 | 36193214 | 36193235 | chr13 | 69934195 | 69934216 |
| chr2 | 50098904 | 50098926 | chr7 | 38601793 | 38601814 | chr14 | 66094377 | 66094398 |
| chr2 | 73265656 | 73265678 | chr7 | 47909042 | 47909063 | chr16 | 14656670 | 14656691 |
| chr20 | 13432273 | 13432295 | chr7 | 748771 | 748792 | chr16 | 87107991 | 87108013 |
| chr20 | 18449036 | 18449057 | chr8 | 136059616 | 136059639 | chr17 | 353076 | 353097 |
| chr20 | 29297358 | 29297379 | chr8 | 38318140 | 38318163 | chr19 | 13768686 | 13768707 |
| chr20 | 32451442 | 32451465 | chr8 | 3984113 | 3984135 | chr2 | 166284580 | 166284602 |
| chr20 | 38350601 | 38350622 | chr9 | 113808805 | 113808826 | chr3 | 10226168 | 10226189 |
| chr20 | 49217595 | 49217616 | chr9 | 116737579 | 116737600 | chr3 | 115343892 | 115343913 |
| chr20 | 64269672 | 64269693 | chr9 | 12496083 | 12496104 | chr3 | 134983639 | 134983660 |
| chr21 | 5738568 | 5738589 | chr9 | 129327837 | 129327858 | chr3 | 178334452 | 178334474 |
| chr21 | 5859152 | 5859173 | chr9 | 132858640 | 132858661 | chr3 | 45909277 | 45909299 |
| chr21 | 7585888 | 7585909 | chr9 | 8978325 | 8978346 | chr3 | 73176881 | 73176902 |
| chr21 | 8136638 | 8136659 | chrX | 134645573 | 134645594 | chr4 | 81742352 | 81742373 |
| chr21 | 8319683 | 8319704 | chrX | 24998312 | 24998335 | chr4 | 8245260 | 8245281 |
| chr3 | 115208357 | 115208380 | chrX | 88238872 | 88238894 | chr5 | 168066527 | 168066548 |
| chr3 | 139072976 | 139072997 | chrY | 15614017 | 15614038 | chr5 | 168085915 | 168085937 |
| chr3 | 30158407 | 30158428 | chr1 | 203223874 | 203223895 | chr5 | 6207756 | 6207778 |
| chr3 | 38760699 | 38760721 | chr1 | 43604017 | 43604038 | chr6 | 149489057 | 149489079 |
| chr3 | 58128755 | 58128776 | chr10 | 107165027 | 107165048 | chr6 | 151771012 | 151771033 |
| chr3 | 59247420 | 59247441 | chr10 | 3981954 | 3981975 | chr6 | 154959101 | 154959124 |
| chr3 | 71889200 | 71889221 | chr11 | 12261472 | 12261494 | chr6 | 71153933 | 71153956 |
| chr3 | 75350195 | 75350217 | chr14_{—} | 28652 | 28674 | chr6 | 95286431 | 95286453 |
| | | | KI270725v1_random | | | | | |
| chr4 | 129368696 | 129368717 | chr15 | 20303769 | 20303791 | chr7 | 151872799 | 151872820 |
| chr4 | 138131152 | 138131173 | chr15 | 21149388 | 21149410 | chr8 | 135291727 | 135291748 |
| chr4 | 145211541 | 145211562 | chr15 | 60232485 | 60232507 | chr8 | 142002619 | 142002641 |
| chr4 | 175410598 | 175410619 | chr15 | 62708764 | 62708787 | chr8 | 22622609 | 22622632 |
| chr4 | 176012819 | 176012840 | chr15 | 69844005 | 69844027 | chr8 | 48402295 | 48402317 |
| chr4 | 23365348 | 23365370 | chr16 | 32301913 | 32301935 | chr8 | 59391289 | 59391312 |
| chr4 | 8051122 | 8051143 | chr16 | 32860233 | 32860255 | chr8 | 74353643 | 74353665 |
| chr5 | 109936561 | 109936582 | chr16 | 33508183 | 33508205 | chr9 | 127215278 | 127215301 |
| chr5 | 154754303 | 154754326 | chr16 | 33546176 | 33546198 | chrX | 102000238 | 102000259 |
| chr5 | 172938372 | 172938393 | chr16 | 34001286 | 34001308 | chrX | 110824137 | 110824158 |
| chr5 | 67123377 | 67123398 | chr16_{—} | 1122344 | 1122366 | chrX | 9484434 | 9484457 |
| | | | KI207028v1_random | | | | | |
| chr6 | 115441973 | 115441996 | chr16_{—} | 1846473 | 1846495 | chrX | 95109255 | 95109277 |
| | | | KI270728v1_random | | | | | |
| chr6 | 128605720 | 128605741 | chr16_{—} | 215852 | 215874 | chr1 | 163429927 | 163429948 |
| | | | KI270728v1_random | | | | | |
| chr6 | 155825911 | 155825933 | chr18 | 12089271 | 12089293 | chr1 | 168165741 | 168165762 |
| chr6 | 28497324 | 28497345 | chr18 | 14172226 | 14172248 | chr1 | 218399183 | 218399204 |
| chr6 | 38687945 | 38687967 | chr19 | 271964 | 271985 | chr1 | 225901558 | 225901579 |
| chr6 | 55890478 | 55890501 | chr19 | 29923127 | 29923148 | chr1 | 239825074 | 239825095 |
| chr6 | 62089908 | 62089929 | chr19 | 39416865 | 39416886 | chr1 | 246176999 | 246177020 |
| chr7 | 118119892 | 118119913 | chr2 | 75306193 | 75306215 | chr1 | 66367983 | 66368005 |
| chr7 | 137243739 | 137243760 | chr2 | 821670 | 821691 | chr10 | 105792310 | 105792331 |
| chr7 | 14322295 | 14322316 | chr20 | 61883165 | 61883187 | chr10 | 119690965 | 119690986 |
| chr7 | 152870674 | 152870695 | chr21 | 14057110 | 14057132 | chr10 | 15629626 | 15629649 |
| chr7 | 55338095 | 55338117 | chr21 | 41498789 | 41498811 | chr10 | 20137117 | 20137140 |
| chr7 | 91189289 | 91189310 | chr21 | 8657234 | 8657256 | chr10 | 56925205 | 56925227 |
| chr8 | 118595807 | 118595830 | chr22 | 10634363 | 10634385 | chr10 | 71769157 | 71769178 |
| chr8 | 21920926 | 21920947 | chr22 | 16529314 | 16529336 | chr10 | 96692195 | 96692217 |
| chr8 | 71336124 | 71336145 | chr22 | 22292514 | 22292536 | chr11 | 11161353 | 11161374 |
| chr8 | 8444382 | 8444403 | chr3 | 154767190 | 154767211 | chr11 | 16266249 | 16266271 |
| chr8 | 90937374 | 90937396 | chr3 | 38522285 | 38522306 | chr11 | 41679797 | 41679819 |
| chr8 | 92334917 | 92334939 | chr3 | 38752218 | 38752240 | chr11 | 91540261 | 91540283 |
| chr9 | 114450778 | 114450799 | chr3 | 62350580 | 62350602 | chr12 | 18730599 | 18730622 |
| chr9 | 122134671 | 122134694 | chr4 | 149562539 | 149562561 | chr12 | 31779884 | 31779905 |
| chr9 | 34403481 | 34403502 | chr4 | 1547652 | 1547673 | chr12 | 51699567 | 51699588 |
| chr9 | 75992624 | 75992645 | chr4 | 1735133 | 1735154 | chr12 | 66038774 | 66038795 |
| chrX | 115546438 | 115546460 | chr5 | 168627963 | 168627985 | chr12 | 83661770 | 83661791 |
| chrX | 53085475 | 53085496 | chr5 | 23795022 | 23795043 | chr13 | 56206698 | 56206720 |
| chrY | 20804459 | 20804480 | chr6 | 142507007 | 142507030 | chr13 | 92515738 | 92515759 |
| chr1 | 245125422 | 245125444 | chr6 | 45418675 | 45418697 | chr13 | 99930308 | 99930329 |
| chr10 | 97153588 | 97153609 | chr7 | 135558534 | 135558555 | chr14 | 100709342 | 100709363 |
| chr11 | 130405683 | 130405704 | chr8 | 124041822 | 124041843 | chr14 | 60948496 | 60948517 |
| chr12 | 41542249 | 41542270 | chr8 | 143306748 | 143306771 | chr14 | 63458373 | 63458394 |
| chr15 | 33405410 | 33405431 | chr8 | 3193432 | 3193453 | chr14 | 84372203 | 84372224 |
| chr15 | 60494975 | 60494996 | chr8 | 8236128 | 8236151 | chr14 | 91094537 | 91094558 |
| chr15 | 77987866 | 77987887 | chr9 | 127698860 | 127698882 | chr15 | 21654639 | 21654661 |
| chr16 | 14924924 | 14924945 | chr9 | 40589872 | 40589894 | chr15 | 22097135 | 22097157 |
| chr16 | 16323575 | 16323596 | chr9 | 63047613 | 63047635 | chr15 | 72236372 | 72236393 |
| chr16 | 16363575 | 16363596 | chr9 | 63390838 | 63390860 | chr15 | 99388177 | 99388200 |
| chr16 | 18345071 | 18345092 | chr9 | 64717354 | 64717376 | chr15_{—} | 388891 | 388913 |
| | | | | | | KI270727v1_random | | |
| chr16 | 18388585 | 18388606 | chr9 | 65201663 | 65201685 | chr16 | 47873889 | 47873910 |
| chr16 | 2112027 | 2112048 | chr9 | 65820018 | 65820040 | chr17 | 49809267 | 49809289 |
| chr16 | 50712259 | 50712280 | chr9 | 93336083 | 93336105 | chr17 | 55088691 | 55088714 |
| chr16 | 87750777 | 87750798 | chr9 | 93515538 | 93515560 | chr17 | 67339549 | 67339570 |
| chr16 | 87750777 | 87750799 | chr9 | 98612376 | 98612398 | chr18 | 32630505 | 32630528 |
| chr17 | 7139560 | 7139583 | chrUn_{—} | 136081 | 136103 | chr18 | 51862106 | 51862127 |
| | | | KI20749v1 | | | | | |
| chr17 | 79173446 | 79173467 | chrX | 134771228 | 134771250 | chr18 | 73818723 | 73818744 |
| chr19 | 41198803 | 41198824 | chrY | 11438592 | 11438614 | chr19 | 18493880 | 18493902 |
| chr2 | 156068013 | 156068034 | chrY | 26403323 | 26403345 | chr19 | 44719395 | 44719417 |
| chr2 | 156548581 | 156548602 | chr1 | 114755705 | 114755728 | chr2 | 125952758 | 125952779 |
| chr2 | 54177434 | 54177455 | chr1 | 80195820 | 80195841 | chr2 | 139799913 | 139799936 |
| chr20 | 15214248 | 15214269 | chr10 | 46094875 | 46094896 | chr2 | 165310320 | 165310342 |
| chr20 | 24606031 | 24606052 | chr10 | 48196282 | 48196304 | chr2 | 166051970 | 166051991 |
| chr20 | 50089991 | 50090013 | chr10 | 80201966 | 80201989 | chr2 | 166303283 | 166303305 |
| chr22 | 39586599 | 39586620 | chr10 | 8311347 | 8311368 | chr2 | 172323440 | 172323461 |
| chr3 | 155889542 | 155889563 | chr10 | 95610284 | 95610306 | chr2 | 179653735 | 179653756 |
| chr3 | 38755941 | 38755963 | chr10 | 99188037 | 99188058 | chr2 | 198281873 | 198281895 |
| chr3 | 39281879 | 39281900 | chr11 | 111447509 | 111447530 | chr2 | 206523132 | 206523155 |
| chr3 | 68859694 | 68859715 | chr11 | 116742303 | 116742326 | chr2 | 211589335 | 211589356 |
| chr4 | 88823295 | 88823316 | chr11 | 117873838 | 117873861 | chr2 | 223811794 | 223811815 |
| chr5 | 103540423 | 103540444 | chr11 | 34171625 | 34171647 | chr2 | 239835025 | 239835048 |
| chr5 | 84165474 | 84165495 | chr11 | 99967904 | 99967925 | chr2 | 85720060 | 85720081 |
| chr5 | 98912731 | 98912753 | chr12 | 10226257 | 10226278 | chr2 | 9888602 | 9888624 |
| chr7 | 139451004 | 139451025 | chr12 | 1993974 | 1993996 | chr20 | 30613675 | 30613696 |
| chr7 | 140617217 | 140617238 | chr12 | 87701468 | 87701489 | chr21 | 9072499 | 9072522 |
| chr8 | 144808058 | 144808079 | chr13 | 22987248 | 22987269 | chr22 | 19036400 | 19036421 |
| chr8 | 42506480 | 42506501 | chr13 | 44594630 | 44594651 | chr22 | 19345323 | 19345344 |
| chr9 | 121268473 | 121268495 | chr13 | 71034293 | 71034316 | chr3 | 118483785 | 118483807 |
| chr9 | 125767930 | 125767952 | chr14 | 18625660 | 18625681 | chr3 | 12201058 | 12201079 |
| chr9 | 15713241 | 15713262 | chr14 | 19689363 | 19689384 | chr3 | 172628542 | 172628563 |
| chr9 | 75081273 | 75081294 | chr14 | 45436755 | 45436777 | chr3 | 187797026 | 187797047 |
| chrX | 20478318 | 20478339 | chr14 | 55804739 | 55804761 | chr3 | 88438945 | 88438966 |
| chr1 | 175444473 | 175444494 | chr15 | 27948284 | 27948307 | chr3 | 99743398 | 99743421 |
| chr1 | 201448312 | 201448333 | chr15 | 47193064 | 47193085 | chr4 | 104263476 | 104263498 |
| chr1 | 201529925 | 201529947 | chr16 | 88282516 | 88282537 | chr4 | 115502767 | 115502789 |
| chr1 | 230112711 | 230112732 | chr17 | 29595944 | 29595966 | chr4 | 149515395 | 149515418 |
| chr1 | 389212 | 389235 | chr17 | 34587857 | 34587878 | chr4 | 156446223 | 156446246 |
| chr10 | 121567501 | 121567522 | chr17 | 66661126 | 66661148 | chr4 | 158560326 | 158560348 |
| chr10 | 64861924 | 64861945 | chr18 | 44282618 | 44282639 | chr4 | 17127046 | 17127068 |
| chr10 | 71724632 | 71724653 | chr18 | 56109483 | 56109504 | chr4 | 36842914 | 36842936 |
| chr10 | 77794367 | 77794389 | chr19 | 41761232 | 41761254 | chr4 | 42212428 | 42212449 |
| chr10 | 78416925 | 78416946 | chr19 | 41763774 | 41763796 | chr4 | 52359436 | 52359457 |
| chr10 | 78961983 | 78962004 | chr19 | 41807094 | 41807116 | chr4 | 71524135 | 71524156 |
| chr11 | 105698987 | 105699010 | chr19 | 42522197 | 42522219 | chr5 | 103440273 | 103440294 |
| chr11 | 116061423 | 116061445 | chr2 | 130007283 | 130007304 | chr5 | 106393920 | 106393941 |
| chr11 | 18934319 | 18934341 | chr2 | 147019337 | 147019360 | chr5 | 112398009 | 112398030 |
| chr11 | 18956546 | 18956568 | chr2 | 166284780 | 166284802 | chr5 | 126349877 | 126349898 |
| chr11 | 47270850 | 47270872 | chr2 | 210461905 | 210461926 | chr5 | 127630710 | 127630733 |
| chr12 | 105642043 | 105642064 | chr2 | 32942717 | 32942738 | chr5 | 134823819 | 134823840 |
| chr12 | 121776377 | 121776398 | chr2 | 88545335 | 88545356 | chr5 | 161450071 | 161450092 |
| chr12 | 130357913 | 130357934 | chr20 | 40816314 | 40816335 | chr5 | 63311092 | 63311113 |
| chr12 | 132765833 | 132765856 | chr20 | 48467757 | 48467778 | chr5 | 68226819 | 68226840 |
| chr12 | 132905329 | 132905350 | chr20 | 59385246 | 59385269 | chr5 | 68263783 | 68263804 |
| chr12 | 3005030 | 3005051 | chr21 | 24973764 | 24973785 | chr5 | 73863907 | 73863928 |
| chr12 | 96129083 | 96129105 | chr22 | 15552714 | 15552735 | chr5 | 793395 | 793416 |
| chr13 | 113429827 | 113429849 | chr22 | 27997215 | 27997236 | chr5 | 85758370 | 85758392 |
| chr13 | 24649311 | 24649333 | chr22 | 47994724 | 47994746 | chr6 | 117895873 | 117895894 |
| chr13 | 49321560 | 49321581 | chr3 | 13362760 | 13362781 | chr6 | 155799843 | 155799866 |
| chr13 | 74288649 | 74288672 | chr3 | 154023880 | 154023902 | chr6 | 169594949 | 169594972 |
| chr13 | 95780988 | 95781009 | chr3 | 165659333 | 165659354 | chr6 | 42170408 | 42170429 |
| chr13 | 99408376 | 99408397 | chr3 | 194382648 | 194382671 | chr6 | 55168056 | 55168078 |
| chr14 | 48035673 | 48035695 | chr3 | 3264339 | 3264360 | chr7 | 128811009 | 128811031 |
| chr14 | 95009472 | 95009494 | chr4 | 147481957 | 147481980 | chr7 | 93930640 | 93930662 |
| chr15 | 31530685 | 31530706 | chr4 | 154549407 | 154549428 | chr8 | 114092319 | 114092341 |
| chr15 | 31552815 | 31552836 | chr4 | 162875012 | 162875035 | chr8 | 125705235 | 125705256 |
| chr15 | 66629204 | 66629227 | chr5 | 101674079 | 101674100 | chr8 | 127177604 | 127177625 |
| chr15 | 69283164 | 69283185 | chr5 | 139982158 | 139982180 | chr8 | 43540940 | 43540961 |
| chr15 | 78195307 | 78195330 | chr5 | 143976245 | 143976266 | chr9 | 115759533 | 115759554 |
| chr16 | 1206137 | 1206159 | chr5 | 21985063 | 21985084 | chr9 | 116410209 | 116410232 |
| chr16 | 1371839 | 1371861 | chr5 | 69994824 | 69994845 | chr9 | 37667360 | 37667381 |
| chr16 | 27489697 | 27489719 | chr5 | 70869901 | 70869922 | chr9 | 83375386 | 83375409 |
| chr16 | 86314798 | 86314819 | chr5 | 82428487 | 82428510 | chrX | 113896423 | 113896444 |
| chr16 | 87000301 | 87000322 | chr6 | 132597537 | 132597558 | chrX | 18803751 | 18803772 |
| chr17 | 19559959 | 19559981 | chr6 | 163177753 | 163177776 | chrX | 19705923 | 19705946 |
| chr17 | 50591468 | 50591490 | chr6 | 23306955 | 23306976 | chrX | 29277535 | 29277556 |
| chr17 | 62397428 | 62397451 | chr6 | 25839497 | 25839518 | chrX | 46428082 | 46428103 |
| chr17 | 65539649 | 65539670 | chr6 | 35732304 | 35732325 | chrX | 86724515 | 86724537 |
| chr18 | 47621706 | 47621727 | chr7 | 136446761 | 136446782 | chrY | 11157940 | 11157963 |
| chr18 | 49303939 | 49303960 | chr7 | 29758402 | 29758423 | chr1 | 110728702 | 110728723 |
| chrl9 | 1432127 | 1432148 | chr7 | 34654518 | 34654540 | chr1 | 111772782 | 111772803 |
| chr19 | 28482476 | 28482497 | chr8 | 107976268 | 107976289 | chr1 | 157253826 | 157253847 |
| chr19 | 3536679 | 3536700 | chr8 | 22618602 | 22618623 | chr1 | 175671514 | 175671535 |
| chr19 | 44622402 | 44622423 | chr8 | 32142917 | 32142938 | chr1 | 220276090 | 220276111 |
| chr19 | 58266924 | 58266945 | chr8 | 72411350 | 72411372 | chr1 | 232902073 | 232902094 |
| chr19 | 58516941 | 58516962 | chr9 | 111442930 | 111442953 | chr1 | 23900289 | 23900310 |
| chr2 | 103159794 | 103159816 | chr9 | 12678667 | 12678688 | chr1 | 23900897 | 23900918 |
| chr2 | 10880753 | 10880774 | chr9 | 129897310 | 129897332 | chr1 | 32770597 | 32770618 |
| chr2 | 109987662 | 109987683 | chr9 | 25693154 | 25693177 | chr1 | 39504712 | 39504734 |
| chr2 | 110383822 | 110383843 | chr9 | 76876314 | 76876335 | chr1 | 46238485 | 46238506 |
| chr2 | 120676215 | 120676237 | chrX | 52819012 | 52819035 | chr1 | 50137891 | 50137912 |
| chr2 | 135885723 | 135885744 | chrY | 13047155 | 13047176 | chr1 | 829401 | 829424 |
| chr2 | 20595609 | 20595631 | chr1 | 116045805 | 116045826 | chr1 | 87979064 | 87979086 |
| chr2 | 236107275 | 236107296 | chr1 | 120524331 | 120524352 | chr10 | 103640573 | 103640594 |
| chr2 | 239351905 | 239351928 | chr1 | 148997917 | 148997938 | chr10 | 14556424 | 14556445 |
| chr2 | 72391531 | 72391553 | chr1 | 151862959 | 151862980 | chr10 | 38653807 | 38653828 |
| chr2 | 74675848 | 74675869 | chr1 | 167440689 | 167440711 | chr10 | 3970671 | 3970692 |
| chr20 | 33197447 | 33197469 | chr1 | 168115603 | 168115624 | chr10 | 42269302 | 42269323 |
| chr21 | 43796715 | 43796736 | chr1 | 16978592 | 16978613 | chr10 | 43355963 | 43355984 |
| chr22 | 26472914 | 26472936 | chr1 | 229636941 | 229636962 | chr10 | 47378440 | 47378461 |
| chr22 | 37940693 | 37940714 | chr1 | 24264602 | 24264623 | chr10 | 81680456 | 81680477 |
| chr22 | 39658965 | 39658986 | chr1 | 44348571 | 44348592 | chr11 | 109342912 | 109342933 |
| chr3 | 102092084 | 102092107 | chr1 | 97897476 | 97897497 | chr11 | 110768898 | 110768919 |
| chr3 | 112104244 | 112104265 | chr10 | 23165804 | 23165826 | chr11 | 112180008 | 112180030 |
| chr3 | 128744556 | 128744578 | chr10 | 63315407 | 63315429 | chr11 | 117348819 | 117348840 |
| chr3 | 129998664 | 129998685 | chr10 | 66751522 | 66751544 | chr11 | 18804780 | 18804801 |
| chr3 | 138079920 | 138079941 | chr10 | 6801553 | 6801574 | chr11 | 62428859 | 62428880 |
| chr3 | 179263349 | 179263370 | chr10 | 98369695 | 98369716 | chr11 | 64466273 | 64466296 |
| chr3 | 38739520 | 38739542 | chr10 | 99617183 | 99617204 | chr11 | 6694240 | 6694261 |
| chr4 | 11149833 | 11149854 | chr12 | 103184001 | 103184022 | chr12 | 110781714 | 110781736 |
| chr4 | 124096134 | 124096155 | chr12 | 112407576 | 112407597 | chr12 | 110781715 | 110781736 |
| chr4 | 146955938 | 146955960 | chr12 | 117070062 | 117070083 | chr12 | 115205603 | 115205624 |
| chr4 | 170026553 | 170026575 | chr12 | 51663013 | 51663035 | chr12 | 127231134 | 127231155 |
| chr5 | 181429655 | 181429678 | chr12 | 59396770 | 59396791 | chr12 | 129670615 | 129670636 |
| chr5 | 21493460 | 21493481 | chr13 | 111941611 | 111941634 | chr12 | 15037363 | 15037384 |
| chr5 | 34180704 | 34180725 | chr13 | 20226701 | 20226723 | chr12 | 52077141 | 52077162 |
| chr5 | 67085319 | 67085340 | chr13 | 31559301 | 31559322 | chr12 | 6450200 | 6450221 |
| chr5 | 82277802 | 82277825 | chr13 | 54350438 | 54350459 | chr13 | 112323893 | 112323916 |
| chr6 | 149983169 | 149983192 | chr14 | 100534376 | 100534398 | chr13 | 67503995 | 67504017 |
| chr6 | 150045094 | 150045117 | chr14 | 103374438 | 103374459 | chr15 | 100202177 | 100202198 |
| chr6 | 170705255 | 170705278 | chr14 | 63612101 | 63612122 | chr15 | 44096819 | 44096840 |
| chr6 | 40326950 | 40326971 | chr14 | 94924114 | 94924137 | chr15 | 94677590 | 94677612 |
| chr7 | 156943597 | 156943618 | chr14 | 97887975 | 97887996 | chr16 | 20465679 | 20465702 |
| chr7 | 157804520 | 157804541 | chr15 | 66786266 | 66786287 | chr16 | 20559262 | 20559285 |
| chr7 | 41188633 | 41188654 | chr15 | 82138605 | 82138627 | chr16 | 32423276 | 32423297 |
| chr8 | 102946 | 102969 | chr15 | 88831872 | 88831894 | chr16 | 53342629 | 53342650 |
| chr8 | 111164312 | 111164334 | chr16 | 26819249 | 26819271 | chr16 | 66650888 | 66650909 |
| chr9 | 111573096 | 111573118 | chr16 | 62282993 | 62283016 | chr16 | 85592850 | 85592871 |
| chr9 | 129078778 | 129078799 | chr16 | 74719101 | 74719122 | chr16 | 87314891 | 87314913 |
| chr9 | 38871672 | 38871693 | chr17 | 33572853 | 33572874 | chr17 | 47655542 | 47655564 |
| chr9 | 62096099 | 62096120 | chr18 | 28806699 | 28806720 | chr17 | 64039697 | 64039720 |
| chr9 | 67492262 | 67492283 | chr18 | 39821341 | 39821363 | chr17 | 64818903 | 64818924 |
| chr9 | 68460951 | 68460972 | chr19 | 15031517 | 15031539 | chr17 | 65106815 | 65106836 |
| chr9 | 76787902 | 76787925 | chr19 | 2884427 | 2884448 | chr17 | 65447014 | 65447035 |
| chrX | 50395284 | 50395305 | chr19 | 54704669 | 54704690 | chr17 | 80882551 | 80882573 |
| chrX | 73763565 | 73763587 | chr2 | 165176189 | 165176211 | chr18 | 1469234 | 1469255 |
| chrX | 91536775 | 91536797 | chr2 | 165296010 | 165296032 | chr18 | 57837950 | 57837971 |
| chrY | 4778305 | 4778327 | chr2 | 166073416 | 166073438 | chr18 | 70384586 | 70384607 |
| chr1 | 1047408 | 1047431 | chr2 | 166311557 | 166311579 | chr19 | 44473808 | 44473829 |
| chr1 | 112272803 | 112272825 | chr2 | 178928872 | 178928893 | chr19 | 52740558 | 52740579 |
| chr1 | 49353896 | 49353917 | chr2 | 205437736 | 205437758 | chr2 | 10064549 | 10064571 |
| chr10 | 14093021 | 14093044 | chr2 | 227782297 | 227782319 | chr2 | 125400498 | 125400520 |
| chr10 | 29199574 | 29199595 | chr2 | 233662547 | 233662569 | chr2 | 136156621 | 136156642 |
| chr11 | 119381098 | 119381119 | chr2 | 35864586 | 35864607 | chr2 | 143967721 | 143967744 |
| chr11 | 65057934 | 65057957 | chr2 | 5417088 | 5417109 | chr2 | 160404642 | 160404664 |
| chr11 | 69777246 | 69777268 | chr2 | 86491497 | 86491518 | chr2 | 166284805 | 166284827 |
| chr12 | 27945151 | 27945172 | chr20 | 14319152 | 14319173 | chr2 | 171274970 | 171274991 |
| chr12 | 44794503 | 44794525 | chr22 | 49344269 | 49344291 | chr2 | 230313351 | 230313373 |
| chr13 | 106331657 | 106331678 | chr3 | 146246154 | 146246175 | chr2 | 2695712 | 2695733 |
| chr14 | 36719207 | 36719228 | chr3 | 168393781 | 168393802 | chr2 | 91895965 | 91895986 |
| chr15 | 31179489 | 31179511 | chr3 | 46155606 | 46155627 | chr20 | 20797859 | 20797880 |
| chr16 | 89566197 | 89566218 | chr4 | 113280490 | 113280512 | chr20 | 24493828 | 24493849 |
| chr17 | 47055562 | 47055583 | chr4 | 64749297 | 64749319 | chr20 | 36236087 | 36236108 |
| chr18 | 78628737 | 78628760 | chr4 | 85568706 | 85568727 | chr20 | 45857529 | 45857550 |
| chr19 | 43302685 | 43302706 | chr5 | 107449451 | 107449473 | chr20 | 48461342 | 48461363 |
| chr2 | 238381749 | 238381770 | chr5 | 121012150 | 121012171 | chr20 | 57807620 | 57807641 |
| chr2 | 49922189 | 49922211 | chr5 | 1326658 | 1326680 | chr20 | 59590440 | 59590461 |
| chr2 | 66528955 | 66528976 | chr5 | 1326687 | 1326709 | chr22 | 16441864 | 16441885 |
| chr2 | 7429233 | 7429255 | chr5 | 79949732 | 79949753 | chr22 | 33423179 | 33423200 |
| chr20 | 10672943 | 10672966 | chr5 | 81104790 | 81104812 | chr22 | 37134063 | 37134085 |
| chr20 | 35503270 | 35503293 | chr5 | 91214887 | 91214908 | chr3 | 123222955 | 123222976 |
| chr20 | 38705488 | 38705509 | chr6 | 152753587 | 152753609 | chr3 | 95912639 | 95912660 |
| chr22 | 29517883 | 29517904 | chr6 | 50510462 | 50510485 | chr4 | 102914035 | 102914056 |
| chr22 | 32199466 | 32199489 | chr6 | 7798609 | 7798630 | chr4 | 137452393 | 137452414 |
| chr3 | 194681445 | 194681466 | chr7 | 145991153 | 145991174 | chr4 | 163945894 | 163945916 |
| chr3 | 25816028 | 25816049 | chr7 | 77347062 | 77347085 | chr4 | 1815215 | 1815236 |
| chr3 | 38793779 | 38793801 | chr7 | 77614538 | 77614560 | chr4 | 20252878 | 20252900 |
| chr3 | 43078355 | 43078376 | chr8 | 58860663 | 58860684 | chr4 | 25487429 | 25487450 |
| chr3 | 5504577 | 5504598 | chrY | 9215006 | 9215027 | chr4 | 41329119 | 41329140 |
| chr4 | 154399772 | 154399793 | chr1 | 153330925 | 153330946 | chr4 | 41330183 | 41330205 |
| chr5 | 18754355 | 18754376 | chr1 | 179068821 | 179068843 | chr5 | 126853309 | 126853330 |
| chr5 | 1876942 | 1876964 | chr1 | 193317003 | 193317025 | chr5 | 135643584 | 135643607 |
| chr5 | 23859463 | 23859486 | chr1 | 201175016 | 201175038 | chr5 | 144484681 | 144484703 |
| chr6 | 131386169 | 131386190 | chr1 | 229761813 | 229761834 | chr5 | 149320088 | 149320109 |
| chr6 | 157848702 | 157848723 | chr1 | 236611925 | 236611946 | chr5 | 34543122 | 34543143 |
| chr7 | 144505858 | 144505879 | chr1 | 44003243 | 44003264 | chr5 | 38374328 | 38374349 |
| chr7 | 28106012 | 28106033 | chr1 | 60484489 | 60484511 | chr5 | 51582987 | 51583008 |
| chr7 | 5261424 | 5261446 | chr1 | 77272589 | 77272611 | chr5 | 51584136 | 51584157 |
| chr7 | 56972149 | 56972171 | chr1 | 99805299 | 99805321 | chr5 | 5684323 | 5684344 |
| chr7 | 63604777 | 63604799 | chr10 | 7234091 | 7234112 | chr5 | 95184977 | 95184998 |
| chr9 | 11380189 | 11380210 | chr11 | 124639215 | 124639237 | chr6 | 10345815 | 10345837 |
| chrX | 74835062 | 74835083 | chr11 | 128565919 | 128565940 | chr6 | 111939969 | 111939990 |
| chr1 | 113789694 | 113789716 | chr11 | 32183988 | 32184009 | chr6 | 144851102 | 144851123 |
| chr1 | 151492228 | 151492250 | chr11 | 98824964 | 98824985 | chr6 | 169660524 | 169660545 |
| chr1 | 17434745 | 17434767 | chr12 | 52539784 | 52539807 | chr6 | 44791613 | 44791636 |
| chr1 | 237232784 | 237232806 | chr13 | 53239100 | 53239123 | chr6 | 54214841 | 54214862 |
| chr1 | 51343936 | 51343958 | chr13 | 67710449 | 67710471 | chr6 | 85108470 | 85108493 |
| chr1 | 62060283 | 62060305 | chr14 | 101542861 | 101542882 | chr7 | 101293356 | 101293377 |
| chr10 | 12202452 | 12202474 | chr14 | 94101963 | 94101986 | chr7 | 101769888 | 101769909 |
| chr10 | 34500129 | 34500150 | chr15 | 53389064 | 53389085 | chr7 | 71436053 | 71436074 |
| chr10 | 908595 | 908617 | chr15 | 59609984 | 59610006 | chr7 | 77258325 | 77258348 |
| chr10 | 97430628 | 97430649 | chr16 | 83718885 | 83718908 | chr8 | 103188339 | 103188360 |
| chr11 | 131800586 | 131800608 | chr17 | 13619613 | 13619634 | chr8 | 58208948 | 58208971 |
| chr11 | 45059654 | 45059675 | chr18 | 1302211 | 1302234 | chr8 | 90410702 | 90410725 |
| chr11 | 46293596 | 46293618 | chr18 | 34382228 | 34382250 | chr9 | 16091078 | 16091101 |
| chr11 | 67532922 | 67532943 | chr2 | 136683476 | 136683499 | chr9 | 87763613 | 87763634 |
| chr11 | 68473701 | 68473723 | chr2 | 166280389 | 166280411 | chr9 | 89771805 | 89771827 |
| chr11 | 76869199 | 76869221 | chr20 | 21609437 | 21609459 | chrX | 114583997 | 114584020 |
| chr11 | 76891737 | 76891759 | chr3 | 15056861 | 15056883 | chrX | 24592372 | 24592393 |
| chr11 | 85780949 | 85780971 | chr3 | 162472197 | 162472218 | chrX | 26644776 | 26644797 |
| chr11 | 88640473 | 88640495 | chr3 | 192144038 | 192144059 | chrX | 55695885 | 55695906 |
| chr11 | 92366842 | 92366863 | chr3 | 193972967 | 193972988 | chrX | 6695896 | 6695917 |
| chr12 | 104031167 | 104031188 | chr3 | 31318637 | 31318658 | chrX | 97311438 | 97311459 |
| chr12 | 131154018 | 131154039 | chr3 | 33928700 | 33928721 | chrX | 9977092 | 9977114 |
| chr12 | 131507086 | 131507107 | chr3 | 66347945 | 66347967 | chrY | 11353457 | 11353478 |
| chr12 | 22210480 | 22210502 | chr4 | 172137983 | 172138005 | chr11 | 9031037 | 9031063 |
| chr12 | 2412249 | 2412270 | chr4 | 37832335 | 37832356 | chr2 | 135142584 | 135142611 |
| chr12 | 37569101 | 37569123 | chr4 | 53708420 | 53708441 | chr2 | 166281710 | 166281737 |
| chr12 | 50209171 | 50209193 | chr5 | 133278354 | 133278375 | chr2 | 166286555 | 166286581 |
| chr12 | 79672940 | 79672961 | chr5 | 155337749 | 155337770 | chr2 | 166286555 | 166286582 |
| chr12 | 93641177 | 93641198 | chr5 | 39782986 | 39783009 | chr4 | 139864810 | 139864837 |
| chr12 | 95672692 | 95672713 | chr5 | 67714762 | 67714784 | chr5 | 163173454 | 163173480 |
| chr13 | 21222393 | 21222415 | chr6 | 35730038 | 35730059 | chr17 | 63959375 | 63959402 |
| chr13 | 24651496 | 24651519 | chr6 | 44811459 | 44811480 | chr2 | 166278250 | 166278277 |
| chr13 | 29651059 | 29651081 | chr7 | 149925597 | 149925618 | chr4 | 46082495 | 46082522 |
| chr13 | 84115549 | 84115570 | chr7 | 152767248 | 152767269 | chr2 | 166284785 | 166284812 |
| chr14 | 34994321 | 34994343 | chr7 | 156408013 | 156408034 | chr5 | 120440047 | 120440073 |
| chr14 | 75554232 | 75554254 | chr7 | 28741171 | 28741192 | chrX | 148562427 | 148562453 |
| chr15 | 36211196 | 36211217 | chr8 | 29989405 | 29989427 | chr19 | 13426131 | 13426157 |
| chr15 | 62122910 | 62122931 | chr8 | 34718643 | 34718664 | chr2 | 166284792 | 166284819 |
| chr15 | 76793946 | 76793968 | chr8 | 43134646 | 43134667 | chr12 | 123248645 | 123248671 |
| chr15 | 98022583 | 98022605 | chr8 | 752973 | 752994 | chr2 | 166284621 | 166284647 |
| chr16 | 17114450 | 17114472 | chr9 | 74554912 | 74554934 | chr2 | 166284621 | 166284648 |
| chr16 | 2398065 | 2398087 | chr9 | 81540528 | 81540550 | chr14 | 38705215 | 38705242 |
| chr16 | 29733409 | 29733431 | chrX | 152649787 | 152649810 | chr2 | 166293356 | 166293383 |
| chr16 | 56488215 | 56488237 | chr1 | 108020984 | 108021006 | chr2 | 165313740 | 165313766 |
| chr16 | 81033918 | 81033940 | chr1 | 157607538 | 157607560 | chr2 | 166293225 | 166293252 |
| chr17 | 22325036 | 22325058 | chr1 | 51964038 | 51964059 | chr11 | 95324148 | 95324174 |
| chr17 | 27938284 | 27938305 | chr1 | 75514790 | 75514811 | chr12 | 107418063 | 107418089 |
| chr17 | 28384097 | 28384119 | chr10 | 48281408 | 48281430 | chr12 | 51699625 | 51699652 |
| chr17 | 45270070 | 45270092 | chr11 | 116241444 | 116241465 | chr13 | 105592802 | 105592828 |
| chr17 | 56133866 | 56133888 | chr11 | 30474472 | 30474493 | chr2 | 165162747 | 165162773 |
| chr18 | 12282949 | 12282971 | chr12 | 129136215 | 129136236 | chr2 | 165310378 | 165310404 |
| chr18 | 38388400 | 38388422 | chr13 | 22491824 | 22491845 | chr2 | 166051907 | 166051933 |
| chr19 | 36769800 | 36769822 | chr13 | 48042239 | 48042260 | chr2 | 166303220 | 166303247 |
| chr19 | 57588377 | 57588398 | chr13 | 76397102 | 76397123 | chr2 | 217662908 | 217662934 |
| chr2 | 162152161 | 162152184 | chr14 | 72859229 | 72859251 | chr2 | 226368493 | 226368519 |
| chr2 | 23872600 | 23872621 | chr14 | 87684513 | 87684536 | chr6 | 68609343 | 68609369 |
| chr2 | 239298584 | 239298606 | chr15 | 39766578 | 39766601 | chr7 | 30750602 | 30750628 |
| chr2 | 46148451 | 46148472 | chr16 | 1615112 | 1615133 | chr2 | 166305804 | 166305831 |
| chr2 | 88190010 | 88190032 | chr16 | 48745003 | 48745024 | chr3 | 38771291 | 38771318 |
| chr20 | 25843288 | 25843310 | chr18 | 57051391 | 57051412 | chr3 | 38752265 | 38752292 |
| chr20 | 26012592 | 26012614 | chr19 | 51986606 | 51986627 | chr7 | 10755206 | 10755234 |
| chr20 | 34453128 | 34453149 | chr19 | 52276544 | 52276565 | chrX | 133006058 | 133006084 |
| chr21 | 42555302 | 42555323 | chr2 | 130730007 | 130730028 | chr3 | 38752213 | 38752240 |
| chr3 | 23131297 | 23131319 | chr2 | 166286321 | 166286343 | chr3 | 38752415 | 38752442 |
| chr3 | 38793741 | 38793763 | chr2 | 184148698 | 184148721 | chr8 | 28147462 | 28147488 |
| chr3 | 39209399 | 39209421 | chr2 | 44473938 | 44473960 | chr3 | 38793739 | 38793766 |
| chr3 | 43298469 | 43298491 | chr2 | 69785973 | 69785994 | chr3 | 38793953 | 38793980 |
| chr4 | 110374339 | 110374361 | chr3 | 100151370 | 100151392 | chr3 | 38750104 | 38750131 |
| chr4 | 137160917 | 137160939 | chr3 | 172830944 | 172830967 | chr11 | 133729424 | 133729450 |
| chr4 | 138259457 | 138259478 | chr3 | 26459710 | 26459731 | chr3 | 38771273 | 38771300 |
| chr4 | 150215252 | 150215274 | chr3 | 38378001 | 38378022 | chr10 | 112503420 | 112503446 |
| chr4 | 17531757 | 17531779 | chr4 | 69868253 | 69868274 | chr3 | 38757064 | 38757091 |
| chr5 | 14120019 | 14120040 | chr4 | 99790429 | 99790450 | chr4 | 175998006 | 175998032 |
| chr5 | 141309428 | 141309450 | chr5 | 101634255 | 101634276 | chr4 | 80646320 | 80646347 |
| chr5 | 16904562 | 16904584 | chr5 | 135085796 | 135085817 | chrX | 13780722 | 13780748 |
| chr5 | 28146632 | 28146653 | chr5 | 173714270 | 173714292 | chr1 | 105835449 | 105835475 |
| chr6 | 104556941 | 104556963 | chr6 | 149482923 | 149482944 | chr16 | 59275323 | 59275349 |
| chr6 | 10698822 | 10698843 | chr6 | 169223323 | 169223344 | chr3 | 38739609 | 38739636 |
| chr6 | 157813372 | 157813395 | chr6 | 73397278 | 73397299 | chr4 | 96421786 | 96421812 |
| chr6 | 30975969 | 30975991 | chr7 | 139412916 | 139412937 | chr7 | 14395432 | 14395458 |
| chr6 | 4531543 | 4531565 | chr7 | 5354372 | 5354393 | chr13 | 45392357 | 45392383 |
| chr6 | 5716528 | 5716550 | chr7 | 7420448 | 7420469 | chr15 | 80242156 | 80242184 |
| chr6 | 73055476 | 73055497 | chr8 | 10154295 | 10154317 | chr3 | 38739575 | 38739602 |

Results

Screening of SpCas9 and SaCas9 gRNAs Targeted to SCN9A and SCN10A in iPSCs

Following two rounds of gRNA screening in iPSCs and sequencing analysis, the mean average cutting efficiency was calculated based on four replicates for each sample looking at total percentage of insertions and deletions (indels) at the predicted cut site of each gRNA. For the purposes of knocking out the SCN9A and SCN10A genes, the average percentage of indels that would result in a frameshift mutation was also calculated. Guide RNAs were ranked on both mean total indel percentage and mean frameshift-causing indel percentage. Guide RNAs are listed in rank order based on mean frameshift-causing indel percentages and a scrambled non-targeting gRNA as well as untreated cells were included as negative controls (Tables 8-11).

TABLE 8
Mean total indel percentage and mean frameshift-causing indel
percentages generated by SpCas9 gRNAs targeting SCN9A in iPSCs

| | | | | | Mean | | Rank |
|---|---|---|---|---|---|---|---|
| | | | | | frameshift- | Rank | based on |
| | | | | Mean total | causing | based on | frameshift- |
| | Target | Target | | indel | indel | total | causing |
| gRNA name | gene | exon | Cas9 | percentage | percentage | indels | indels |
| Scn9a_Sp_Exon_11_T9 | SCN9A | 11 | SpCas9 | 81.86% | 76.55% | 3 | 1 |
| Scn9a_Sp_Exon_12_T25 | SCN9A | 12 | SpCas9 | 80.50% | 76.05% | 4 | 2 |
| Scn9a_Sp_Exon_11_T6 | SCN9A | 11 | SpCas9 | 78.51% | 74.56% | 5 | 3 |
| Scn9a_Sp_Exon_11_T11 | SCN9A | 11 | SpCas9 | 83.29% | 73.18% | 1 | 4 |
| Scn9a_Sp_Exon_12_T20 | SCN9A | 12 | SpCas9 | 81.94% | 73.08% | 2 | 5 |
| Scn9a_Sp_Exon_11_T14 | SCN9A | 11 | SpCas9 | 76.90% | 72.51% | 8 | 6 |
| Scn9a_Sp_Exon_11_T13 | SCN9A | 11 | SpCas9 | 78.01% | 70.14% | 7 | 7 |
| Scn9a_Sp_Exon_14_T1 | SCN9A | 14 | SpCas9 | 75.27% | 69.46% | 9 | 8 |
| Scn9a_Sp_Exon_7_T5 | SCN9A | 7 | SpCas9 | 74.45% | 69.19% | 11 | 9 |
| Scn9a_Sp_Exon_2_T10 | SCN9A | 2 | SpCas9 | 73.68% | 69.08% | 12 | 10 |
| Scn9a_Sp_Exon_12_T11 | SCN9A | 12 | SpCas9 | 71.34% | 69.01% | 16 | 11 |
| Scn9a_Sp_Exon_12_T17 | SCN9A | 12 | SpCas9 | 75.20% | 68.85% | 10 | 12 |
| Scn9a_Sp_Exon_12_T12 | SCN9A | 12 | SpCas9 | 70.60% | 67.13% | 17 | 13 |
| Scn9a_Sp_Exon_9_T10 | SCN9A | 9 | SpCas9 | 70.41% | 66.70% | 18 | 14 |
| Scn9a_Sp_Exon_11_T2 | SCN9A | 11 | SpCas9 | 72.32% | 64.90% | 13 | 15 |
| Scn9a_Sp_Exon_15_T1 | SCN9A | 15 | SpCas9 | 70.07% | 64.78% | 19 | 16 |
| Scn9a_Sp_Exon_9_T14 | SCN9A | 9 | SpCas9 | 66.06% | 60.75% | 27 | 17 |
| Scn9a_Sp_Exon 12_T28 | SCN9A | 12 | SpCas9 | 68.39% | 60.72% | 20 | 18 |
| Scn9a_Sp_Exon_12_T13 | SCN9A | 12 | SpCas9 | 64.19% | 60.35% | 31 | 19 |
| Scn9a_Sp_Exon_11_T5 | SCN9A | 11 | SpCas9 | 71.86% | 59.88% | 15 | 20 |
| Scn9a_Sp_Exon_12_T24 | SCN9A | 12 | SpCas9 | 68.22% | 58.77% | 22 | 21 |
| Scn9a_Sp_Exon_2_T2 | SCN9A | 2 | SpCas9 | 62.59% | 58.70% | 35 | 22 |
| Scn9a_Sp_Exon_12_T21 | SCN9A | 12 | SpCas9 | 78.19% | 58.69% | 6 | 23 |
| Scn9a_Sp_Exon 12_T27 | SCN9A | 12 | SpCas9 | 67.28% | 58.34% | 25 | 24 |
| Scn9a_Sp_Exon_9_T11 | SCN9A | 9 | SpCas9 | 63.22% | 57.35% | 34 | 25 |
| Scn9a_Sp_Exon_12_T1 | SCN9A | 12 | SpCas9 | 61.57% | 57.18% | 37 | 26 |
| Scn9a_Sp_Exon_12_T23 | SCN9A | 12 | SpCas9 | 64.98% | 56.37% | 29 | 27 |
| Scn9a_Sp_Exon_14_T3 | SCN9A | 14 | SpCas9 | 68.34% | 55.35% | 21 | 28 |
| Scn9a_Sp_Exon_10_T2 | SCN9A | 10 | SpCas9 | 64.20% | 54.01% | 30 | 29 |
| Scn9a_Sp_Exon_12_T4 | SCN9A | 12 | SpCas9 | 61.66% | 53.77% | 36 | 30 |
| Scn9a_Sp_Exon_12_T8 | SCN9A | 12 | SpCas9 | 59.99% | 53.24% | 41 | 31 |
| Scn9a_Sp_Exon_11_T10 | SCN9A | 11 | SpCas9 | 59.73% | 52.88% | 42 | 32 |
| Scn9a_Sp_Exon_2_T14 | SCN9A | 2 | SpCas9 | 63.45% | 52.60% | 32 | 33 |
| Scn9a_Sp_Exon_5_T3 | SCN9A | 5 | SpCas9 | 56.95% | 51.72% | 49 | 34 |
| Scn9a_Sp_Exon_12_T2 | SCN9A | 12 | SpCas9 | 57.05% | 51.39% | 48 | 35 |
| Scn9a_Sp_Exon_11_T15 | SCN9A | 11 | SpCas9 | 67.51% | 51.11% | 24 | 36 |
| Scn9a_Sp_Exon_11_T3 | SCN9A | 11 | SpCas9 | 54.46% | 50.05% | 54 | 37 |
| Scn9a_Sp_Exon_9_T6 | SCN9A | 9 | SpCas9 | 63.44% | 49.88% | 33 | 38 |
| Scn9a_Sp_Exon_9_T5 | SCN9A | 9 | SpCas9 | 53.89% | 49.71% | 56 | 39 |
| Scn9a_Sp_Exon_7_T2 | SCN9A | 7 | SpCas9 | 54.86% | 47.44% | 52 | 40 |
| Scn9a_Sp_Exon_2_T4 | SCN9A | 2 | SpCas9 | 57.70% | 46.83% | 47 | 41 |
| Scn9a_Sp_Exon_2_T1 | SCN9A | 2 | SpCas9 | 66.37% | 45.78% | 26 | 42 |
| Scn9a_Sp_Exon_9_T2 | SCN9A | 9 | SpCas9 | 58.26% | 45.73% | 46 | 43 |
| Scn9a_Sp_Exon_4_T5 | SCN9A | 4 | SpCas9 | 49.69% | 45.71% | 62 | 44 |
| Scn9a_Sp_Exon_7_T6 | SCN9A | 7 | SpCas9 | 60.22% | 45.52% | 40 | 45 |
| Scn9a_Sp_Exon_12_T10 | SCN9A | 12 | SpCas9 | 53.94% | 45.27% | 55 | 46 |
| Scn9a_Sp_Exon_4_T3 | SCN9A | 4 | SpCas9 | 50.92% | 45.04% | 58 | 47 |
| Scn9a_Sp_Exon_11_T12 | SCN9A | 11 | SpCas9 | 59.05% | 43.60% | 45 | 48 |
| Scn9a_Sp_Exon_9_T13 | SCN9A | 9 | SpCas9 | 60.77% | 43.49% | 39 | 49 |
| Scn9a_Sp_Exon_11_T7 | SCN9A | 11 | SpCas9 | 50.76% | 43.49% | 59 | 50 |
| Scn9a_Sp_Exon_15_T2 | SCN9A | 15 | SpCas9 | 59.54% | 43.34% | 44 | 51 |
| Scn9a_Sp_Exon_12_T16 | SCN9A | 12 | SpCas9 | 61.29% | 42.92% | 38 | 52 |
| Scn9a_Sp_Exon_12_T19 | SCN9A | 12 | SpCas9 | 59.59% | 42.45% | 43 | 53 |
| Scn9a_Sp_Exon_2_T15 | SCN9A | 2 | SpCas9 | 44.47% | 41.45% | 67 | 54 |
| Scn9a_Sp_Exon_14_T2 | SCN9A | 14 | SpCas9 | 46.19% | 40.97% | 64 | 55 |
| Scn9a_Sp_Exon_2_T13 | SCN9A | 2 | SpCas9 | 55.38% | 40.79% | 51 | 56 |
| Scn9a_Sp_Exon_9_T12 | SCN9A | 9 | SpCas9 | 44.16% | 39.70% | 68 | 57 |
| Scn9a_Sp_Exon_9_T4 | SCN9A | 9 | SpCas9 | 50.13% | 39.54% | 61 | 58 |
| Scn9a_Sp_Exon_2_T11 | SCN9A | 2 | SpCas9 | 53.70% | 39.17% | 57 | 59 |
| Scn9a_Sp_Exon_10_T5 | SCN9A | 10 | SpCas9 | 55.73% | 38.73% | 50 | 60 |
| Scn9a_Sp_Exon_5_T1 | SCN9A | 5 | SpCas9 | 66.04% | 37.17% | 28 | 61 |
| Scn9a_Sp_Exon 12_T7 | SCN9A | 12 | SpCas9 | 72.23% | 37.12% | 14 | 62 |
| Scn9a_Sp_Exon_7_T1 | SCN9A | 7 | SpCas9 | 50.50% | 37.01% | 60 | 63 |
| Scn9a_Sp_Exon_15_T3 | SCN9A | 15 | SpCas9 | 54.50% | 36.77% | 53 | 64 |
| Scn9a_Sp_Exon_12_T3 | SCN9A | 12 | SpCas9 | 42.40% | 35.16% | 71 | 65 |
| Scn9a_Sp_Exon_7_T3 | SCN9A | 7 | SpCas9 | 46.33% | 34.84% | 63 | 66 |
| Scn9a_Sp_Exon_9_T9 | SCN9A | 9 | SpCas9 | 38.80% | 34.59% | 77 | 67 |
| Scn9a_Sp_Exon_5_T2 | SCN9A | 5 | SpCas9 | 39.80% | 34.24% | 74 | 68 |
| Scn9a_Sp_Exon_2_T9 | SCN9A | 2 | SpCas9 | 45.47% | 33.41% | 65 | 69 |
| Scn9a_Sp_Exon 12_T9 | SCN9A | 12 | SpCas9 | 38.68% | 33.01% | 79 | 70 |
| Scn9a_Sp_Exon_12_T5 | SCN9A | 12 | SpCas9 | 33.67% | 31.96% | 82 | 71 |
| Scn9a_Sp_Exon_14_T5 | SCN9A | 14 | SpCas9 | 44.55% | 31.78% | 66 | 72 |
| Scn9a_Sp_Exon_2_T8 | SCN9A | 2 | SpCas9 | 38.76% | 31.73% | 78 | 73 |
| Scn9a_Sp_Exon_9_T15 | SCN9A | 9 | SpCas9 | 39.98% | 31.56% | 73 | 74 |
| Scn9a_Sp_Exon 12_T14 | SCN9A | 12 | SpCas9 | 33.90% | 31.22% | 81 | 75 |
| Scn9a_Sp_Exon_2_T12 | SCN9A | 2 | SpCas9 | 39.76% | 31.02% | 75 | 76 |
| Scn9a_Sp_Exon 12_T18 | SCN9A | 12 | SpCas9 | 68.21% | 30.76% | 23 | 77 |
| Scn9a_Sp_Exon 12_T15 | SCN9A | 12 | SpCas9 | 39.07% | 30.33% | 76 | 78 |
| Scn9a_Sp_Exon_12_T26 | SCN9A | 12 | SpCas9 | 43.78% | 30.13% | 70 | 79 |
| Scn9a_Sp_Exon_5_T4 | SCN9A | 5 | SpCas9 | 38.57% | 27.51% | 80 | 80 |
| Scn9a_Sp_Exon_12_T6 | SCN9A | 12 | SpCas9 | 31.83% | 26.92% | 84 | 81 |
| Scn9a_Sp_Exon_9_T1 | SCN9A | 9 | SpCas9 | 40.20% | 25.44% | 72 | 82 |
| Scn9a_Sp_Exon_2_T7 | SCN9A | 2 | SpCas9 | 33.06% | 25.35% | 83 | 83 |
| Scn9a_Sp_Exon_4_T7 | SCN9A | 4 | SpCas9 | 26.35% | 24.78% | 88 | 84 |
| Scn9a_Sp_Exon_4_T1 | SCN9A | 4 | SpCas9 | 28.34% | 23.77% | 86 | 85 |
| Scn9a_Sp_Exon_2_T3 | SCN9A | 2 | SpCas9 | 44.01% | 23.46% | 69 | 86 |
| Scn9a_Sp_Exon_12_T22 | SCN9A | 12 | SpCas9 | 24.59% | 22.80% | 92 | 87 |
| Scn9a_Sp_Exon_14_T4 | SCN9A | 14 | SpCas9 | 27.07% | 22.67% | 87 | 88 |
| Scn9a_Sp_Exon_4_T2 | SCN9A | 4 | SpCas9 | 25.94% | 22.12% | 89 | 89 |
| Scn9a_Sp_Exon_4_T4 | SCN9A | 4 | SpCas9 | 24.59% | 19.81% | 93 | 90 |
| Scn9a_Sp_Exon_2_T5 | SCN9A | 2 | SpCas9 | 21.60% | 19.28% | 95 | 91 |
| Scn9a_Sp_Exon_9_T3 | SCN9A | 9 | SpCas9 | 24.82% | 18.98% | 90 | 92 |
| Scn9a_Sp_Exon_9_T8 | SCN9A | 9 | SpCas9 | 31.39% | 18.79% | 85 | 93 |
| Scn9a_Sp_Exon_10_T4 | SCN9A | 10 | SpCas9 | 22.68% | 16.55% | 94 | 94 |
| Scn9a_Sp_Exon_9_T7 | SCN9A | 9 | SpCas9 | 24.72% | 15.94% | 91 | 95 |
| Scn9a_Sp_Exon_14_T6 | SCN9A | 14 | SpCas9 | 17.72% | 12.91% | 96 | 96 |
| Scn9a_Sp_Exon_4_T6 | SCN9A | 4 | SpCas9 | 9.53% | 7.42% | 97 | 97 |
| Scn9a_Sp_Exon_2_T6 | SCN9A | 2 | SpCas9 | 7.09% | 5.35% | 98 | 98 |
| Scn9a_Sp_Exon_10_T1 | SCN9A | 10 | SpCas9 | 1.29% | 1.22% | 99 | 99 |
| Scrambled control | N/A | N/A | SpCas9 | 0.27% | 0.23% | N/A | N/A |
| Untreated | N/A | N/A | N/A | 0.18% | 0.16% | N/A | N/A |

TABLE 9
Mean total indel percentage and mean frameshift-causing indel
percentages generated by SaCas9 gRNAs targeting SCN9A in iPSCs

| | | | | | Mean | | Rank |
|---|---|---|---|---|---|---|---|
| | | | | | frameshift- | Rank | based on |
| | | | | Mean total | causing | based on | frameshift- |
| | Target | Target | | indel | indel | total | causing |
| gRNA name | gene | exon | Cas9 | percentage | percentage | indels | indels |
| Scn9a_Sa_Exon_11_T3 | SCN9A | 11 | SaCas9 | 55.50% | 49.49% | 2 | 1 |
| Scn9a_Sa_Exon_12_T2 | SCN9A | 12 | SaCas9 | 61.96% | 49.44% | 1 | 2 |
| Scn9a_Sa_Exon_5_T6 | SCN9A | 5 | SaCas9 | 54.10% | 42.37% | 3 | 3 |
| Scn9a_Sa_Exon_9_T3 | SCN9A | 9 | SaCas9 | 48.41% | 34.60% | 4 | 4 |
| Scn9a_Sa_Exon_13_T3 | SCN9A | 13 | SaCas9 | 42.29% | 33.08% | 5 | 5 |
| Scn9a_Sa_Exon_12_T1 | SCN9A | 12 | SaCas9 | 33.66% | 28.42% | 7 | 6 |
| Scn9a_Sa_Exon_7_T1 | SCN9A | 7 | SaCas9 | 30.86% | 25.39% | 8 | 7 |
| Scn9a_Sa_Exon_9_T2 | SCN9A | 9 | SaCas9 | 27.99% | 24.43% | 11 | 8 |
| Scn9a_Sa_Exon_15_T3 | SCN9A | 15 | SaCas9 | 28.33% | 23.51% | 10 | 9 |
| Scn9a_Sa_Exon_9_T1 | SCN9A | 9 | SaCas9 | 41.10% | 21.52% | 6 | 10 |
| Scn9a_Sa_Exon_12_T3 | SCN9A | 12 | SaCas9 | 24.81% | 20.49% | 16 | 11 |
| Scn9a_Sa_Exon_7_T5 | SCN9A | 7 | SaCas9 | 25.26% | 20.32% | 15 | 12 |
| Scn9a_Sa_Exon_14_T1 | SCN9A | 14 | SaCas9 | 20.70% | 19.06% | 20 | 13 |
| Scn9a_Sa_Exon_2_T2 | SCN9A | 2 | SaCas9 | 23.85% | 18.85% | 18 | 14 |
| Scn9a_Sa_Exon_11_T1 | SCN9A | 11 | SaCas9 | 26.58% | 17.84% | 14 | 15 |
| Scn9a_Sa_Exon_8_T2 | SCN9A | 8 | SaCas9 | 27.04% | 16.09% | 13 | 16 |
| Scn9a_Sa_Exon_9_T4 | SCN9A | 9 | SaCas9 | 21.90% | 15.56% | 19 | 17 |
| Scn9a_Sa_Exon_15_T6 | SCN9A | 15 | SaCas9 | 19.09% | 15.54% | 22 | 18 |
| Scn9a_Sa_Exon_12_T4 | SCN9A | 12 | SaCas9 | 28.51% | 14.30% | 9 | 19 |
| Scn9a_Sa_Exon_5_T1 | SCN9A | 5 | SaCas9 | 17.45% | 14.16% | 23 | 20 |
| Scn9a_Sa_Exon_5_T3 | SCN9A | 5 | SaCas9 | 16.87% | 13.67% | 24 | 21 |
| Scn9a_Sa_Exon_11_T4 | SCN9A | 11 | SaCas9 | 14.96% | 12.34% | 26 | 22 |
| Scn9a_Sa_Exon_5_T2 | SCN9A | 5 | SaCas9 | 20.59% | 10.46% | 21 | 23 |
| Scn9a_Sa_Exon_7_T6 | SCN9A | 7 | SaCas9 | 12.55% | 9.60% | 27 | 24 |
| Scn9a_Sa_Exon_11_T6 | SCN9A | 11 | SaCas9 | 27.75% | 9.21% | 12 | 25 |
| Scn9a_Sa_Exon_12_T5 | SCN9A | 12 | SaCas9 | 16.55% | 8.77% | 25 | 26 |
| Scn9a_Sa_Exon_4_T4 | SCN9A | 4 | SaCas9 | 9.89% | 8.22% | 30 | 27 |
| Scn9a_Sa_Exon_11_T2 | SCN9A | 11 | SaCas9 | 24.15% | 6.75% | 17 | 28 |
| Scn9a_Sa_Exon_13_T1 | SCN9A | 13 | SaCas9 | 9.03% | 6.63% | 32 | 29 |
| Scn9a_Sa_Exon_10_T2 | SCN9A | 10 | SaCas9 | 12.12% | 6.59% | 29 | 30 |
| Scn9a_Sa_Exon_10_T1 | SCN9A | 10 | SaCas9 | 8.74% | 6.55% | 33 | 31 |
| Scn9a_Sa_Exon_2_T1 | SCN9A | 2 | SaCas9 | 9.69% | 6.54% | 31 | 32 |
| Scn9a_Sa_Exon_5_T4 | SCN9A | 5 | SaCas9 | 12.16% | 6.43% | 28 | 33 |
| Scn9a_Sa_Exon_4_T7 | SCN9A | 4 | SaCas9 | 7.54% | 6.35% | 36 | 34 |
| Scn9a_Sa_Exon_15_T5 | SCN9A | 15 | SaCas9 | 8.00% | 5.84% | 35 | 35 |
| Scn9a_Sa_Exon_5_T5 | SCN9A | 5 | SaCas9 | 7.19% | 5.07% | 37 | 36 |
| Scn9a_Sa_Exon_14_T2 | SCN9A | 14 | SaCas9 | 6.97% | 4.96% | 39 | 37 |
| Scn9a_Sa_Exon_6_T5 | SCN9A | 6 | SaCas9 | 5.78% | 4.90% | 42 | 38 |
| Scn9a_Sa_Exon_15_T7 | SCN9A | 15 | SaCas9 | 8.29% | 4.68% | 34 | 39 |
| Scn9a_Sa_Exon_13_T4 | SCN9A | 13 | SaCas9 | 5.71% | 4.33% | 44 | 40 |
| Scn9a_Sa_Exon_5_T7 | SCN9A | 5 | SaCas9 | 6.54% | 4.33% | 40 | 41 |
| Scn9a_Sa_Exon_14_T6 | SCN9A | 14 | SaCas9 | 5.88% | 4.17% | 41 | 42 |
| Scn9a_Sa_Exon_4_T6 | SCN9A | 4 | SaCas9 | 7.13% | 4.00% | 38 | 43 |
| Scn9a_Sa_Exon_13_T2 | SCN9A | 13 | SaCas9 | 4.50% | 3.54% | 46 | 44 |
| Scn9a_Sa_Exon_4_T5 | SCN9A | 4 | SaCas9 | 5.75% | 3.42% | 43 | 45 |
| Scn9a_Sa_Exon_10_T3 | SCN9A | 10 | SaCas9 | 4.96% | 3.34% | 45 | 46 |
| Scn9a_Sa_Exon_8_T1 | SCN9A | 8 | SaCas9 | 3.76% | 3.00% | 48 | 47 |
| Scn9a_Sa_Exon_7_T4 | SCN9A | 7 | SaCas9 | 4.43% | 2.72% | 47 | 48 |
| Scn9a_Sa_Exon_15_T4 | SCN9A | 15 | SaCas9 | 3.31% | 2.53% | 49 | 49 |
| Scn9a_Sa_Exon_11_T5 | SCN9A | 11 | SaCas9 | 1.53% | 1.28% | 51 | 50 |
| Scn9a_Sa_Exon_15_T1 | SCN9A | 15 | SaCas9 | 1.69% | 1.17% | 50 | 51 |
| Scn9a_Sa_Exon_3_T1 | SCN9A | 3 | SaCas9 | 1.40% | 1.05% | 52 | 52 |
| Scn9a_Sa_Exon_15_T2 | SCN9A | 15 | SaCas9 | 1.27% | 0.83% | 53 | 53 |
| Scn9a_Sa_Exon_2_T3 | SCN9A | 2 | SaCas9 | 1.18% | 0.79% | 54 | 54 |
| Scn9a_Sa_Exon_14_T7 | SCN9A | 14 | SaCas9 | 0.54% | 0.48% | 56 | 55 |
| Scn9a_Sa_Exon_4_T1 | SCN9A | 4 | SaCas9 | 0.40% | 0.40% | 57 | 56 |
| Scn9a_Sa_Exon_6_T2 | SCN9A | 6 | SaCas9 | 0.27% | 0.24% | 60 | 57 |
| Scn9a_Sa_Exon_7_T7 | SCN9A | 7 | SaCas9 | 0.26% | 0.22% | 61 | 58 |
| Scn9a_Sa_Exon_7_T2 | SCN9A | 7 | SaCas9 | 0.27% | 0.22% | 59 | 59 |
| Scn9a_Sa_Exon_7_T8 | SCN9A | 7 | SaCas9 | 0.22% | 0.21% | 62 | 60 |
| Scn9a_Sa_Exon_14_T4 | SCN9A | 14 | SaCas9 | 0.96% | 0.20% | 55 | 61 |
| Scn9a_Sa_Exon_14_T8 | SCN9A | 14 | SaCas9 | 0.31% | 0.18% | 58 | 62 |
| Scn9a_Sa_Exon_14_T10 | SCN9A | 14 | SaCas9 | 0.16% | 0.15% | 64 | 63 |
| Scn9a_Sa_Exon_14_T3 | SCN9A | 14 | SaCas9 | 0.17% | 0.14% | 63 | 64 |
| Scn9a_Sa_Exon_2_T4 | SCN9A | 2 | SaCas9 | 0.15% | 0.10% | 65 | 65 |
| Scn9a_Sa_Exon_8_T3 | SCN9A | 8 | SaCas9 | 0.09% | 0.09% | 66 | 66 |
| Scn9a_Sa_Exon_14_T5 | SCN9A | 14 | SaCas9 | 0.04% | 0.04% | 67 | 67 |
| Scn9a_Sa_Exon_14_T9 | SCN9A | 14 | SaCas9 | 0.02% | 0.02% | 68 | 68 |
| Scrambled control | N/A | N/A | SaCas9 | 0.27% | 0.23% | N/A | N/A |
| Untreated | N/A | N/A | N/A | 0.18% | 0.16% | N/A | N/A |

TABLE 10
Mean total indel percentage and mean frameshift-causing indel
percentages generated by SpCas9 gRNAs targeting SCN10A in iPSCs

| | | | | | Mean | | Rank |
|---|---|---|---|---|---|---|---|
| | | | | | frameshift- | Rank | based on |
| | | | | Mean total | causing | based on | frameshift- |
| | Target | Target | | indel | indel | total | causing |
| gRNA name | gene | exon | Cas9 | percentage | percentage | indels | indels |
| Scn10a_Sp_Exon_1_T15 | SCN10A | 1 | SpCas9 | 75.77% | 73.46% | 1 | 1 |
| Scn10a_Sp_Exon_9_T2 | SCN10A | 9 | SpCas9 | 70.79% | 67.64% | 5 | 2 |
| Scn10a_Sp_Exon_14_T3 | SCN10A | 14 | SpCas9 | 75.21% | 67.26% | 2 | 3 |
| Scn10a_Sp_Exon_10_T7 | SCN10A | 10 | SpCas9 | 69.16% | 66.79% | 7 | 4 |
| Scn10a_Sp_Exon_7_T6 | SCN10A | 7 | SpCas9 | 69.47% | 65.31% | 6 | 5 |
| Scn10a_Sp_Exon_1_T30 | SCN10A | 1 | SpCas9 | 68.89% | 63.21% | 8 | 6 |
| Scn10a_Sp_Exon_11_T10 | SCN10A | 11 | SpCas9 | 72.63% | 62.38% | 3 | 7 |
| Scn10a_Sp_Exon_1_T17 | SCN10A | 1 | SpCas9 | 64.42% | 61.62% | 18 | 8 |
| Scn10a_Sp_Exon_4_T2 | SCN10A | 4 | SpCas9 | 66.31% | 61.10% | 12 | 9 |
| Scn10a_Sp_Exon_10_T3 | SCN10A | 10 | SpCas9 | 66.83% | 60.57% | 9 | 10 |
| Scn10a_Sp_Exon_8_T3 | SCN10A | 8 | SpCas9 | 66.35% | 59.76% | 11 | 11 |
| Scn10a_Sp_Exon_1_T18 | SCN10A | 1 | SpCas9 | 65.19% | 59.51% | 15 | 12 |
| Scn10a_Sp_Exon_1_T16 | SCN10A | 1 | SpCas9 | 66.52% | 59.43% | 10 | 13 |
| Scn10a_Sp_Exon_11_T24 | SCN10A | 11 | SpCas9 | 62.43% | 58.84% | 24 | 14 |
| Scn10a_Sp_Exon_12_T3 | SCN10A | 12 | SpCas9 | 64.78% | 58.60% | 17 | 15 |
| Scn10a_Sp_Exon_8_T8 | SCN10A | 8 | SpCas9 | 65.43% | 58.01% | 14 | 16 |
| Scn10a_Sp_Exon_4_T4 | SCN10A | 4 | SpCas9 | 61.86% | 57.86% | 25 | 17 |
| Scn10a_Sp_Exon_1_T14 | SCN10A | 1 | SpCas9 | 63.17% | 57.59% | 22 | 18 |
| Scn10a_Sp_Exon_14_T1 | SCN10A | 14 | SpCas9 | 71.62% | 56.93% | 4 | 19 |
| Scn10a_Sp_Exon_13_T4 | SCN10A | 13 | SpCas9 | 61.76% | 55.63% | 26 | 20 |
| Scn10a_Sp_Exon_8_T10 | SCN10A | 8 | SpCas9 | 60.22% | 54.64% | 33 | 21 |
| Scn10a_Sp_Exon_7_T2 | SCN10A | 7 | SpCas9 | 60.19% | 54.34% | 34 | 22 |
| Scn10a_Sp_Exon_13_T5 | SCN10A | 13 | SpCas9 | 57.14% | 53.58% | 44 | 23 |
| Scn10a_Sp_Exon_10_T2 | SCN10A | 10 | SpCas9 | 58.03% | 53.29% | 38 | 24 |
| Scn10a_Sp_Exon_7_T7 | SCN10A | 7 | SpCas9 | 56.45% | 52.36% | 48 | 25 |
| Scn10a_Sp_Exon_13_T17 | SCN10A | 13 | SpCas9 | 56.62% | 51.99% | 47 | 26 |
| Scn10a_Sp_Exon_9_T14 | SCN10A | 9 | SpCas9 | 63.41% | 51.82% | 20 | 27 |
| Scn10a_Sp_Exon_1_T25 | SCN10A | 1 | SpCas9 | 60.51% | 51.36% | 32 | 28 |
| Scn10a_Sp_Exon_12_T6 | SCN10A | 12 | SpCas9 | 56.11% | 50.72% | 50 | 29 |
| Scn10a_Sp_Exon_12_T8 | SCN10A | 12 | SpCas9 | 57.60% | 50.69% | 41 | 30 |
| Scn10a_Sp_Exon_2_T3 | SCN10A | 2 | SpCas9 | 63.38% | 50.51% | 21 | 31 |
| Scn10a_Sp_Exon_13_T18 | SCN10A | 13 | SpCas9 | 57.69% | 49.95% | 40 | 32 |
| Scn10a_Sp_Exon_11_T14 | SCN10A | 11 | SpCas9 | 59.24% | 49.80% | 36 | 33 |
| Scn10a_Sp_Exon_11_T19 | SCN10A | 11 | SpCas9 | 53.70% | 49.80% | 67 | 34 |
| Scn10a_Sp_Exon_13_T9 | SCN10A | 13 | SpCas9 | 65.73% | 49.73% | 13 | 35 |
| Scn10a_Sp_Exon_8_T1 | SCN10A | 8 | SpCas9 | 56.68% | 49.55% | 46 | 36 |
| Scn10a_Sp_Exon_1_T10 | SCN10A | 1 | SpCas9 | 54.89% | 49.53% | 60 | 37 |
| Scn10a_Sp_Exon_2_T7 | SCN10A | 2 | SpCas9 | 60.11% | 49.29% | 35 | 38 |
| Scn10a_Sp_Exon_11_T25 | SCN10A | 11 | SpCas9 | 62.96% | 49.26% | 23 | 39 |
| Scn10a_Sp_Exon_1_T3 | SCN10A | 1 | SpCas9 | 55.92% | 48.35% | 51 | 40 |
| Scn10a_Sp_Exon_14_T2 | SCN10A | 14 | SpCas9 | 57.55% | 48.19% | 42 | 41 |
| Scn10a_Sp_Exon_10_T10 | SCN10A | 10 | SpCas9 | 52.18% | 48.08% | 77 | 42 |
| Scn10a_Sp_Exon_13_T13 | SCN10A | 13 | SpCas9 | 54.65% | 47.73% | 61 | 43 |
| Scn10a_Sp_Exon_11_T28 | SCN10A | 11 | SpCas9 | 60.69% | 47.45% | 31 | 44 |
| Scn10a_Sp_Exon_5_T4 | SCN10A | 5 | SpCas9 | 50.66% | 47.09% | 87 | 45 |
| Scn10a_Sp_Exon_9_T3 | SCN10A | 9 | SpCas9 | 51.41% | 46.55% | 81 | 46 |
| Scn10a_Sp_Exon_2_T5 | SCN10A | 2 | SpCas9 | 50.44% | 46.45% | 88 | 47 |
| Scn10a_Sp_Exon_5_T1 | SCN10A | 5 | SpCas9 | 52.12% | 46.30% | 78 | 48 |
| Scn10a_Sp_Exon_11_T16 | SCN10A | 11 | SpCas9 | 60.99% | 45.94% | 30 | 49 |
| Scn10a_Sp_Exon_11_T18 | SCN10A | 11 | SpCas9 | 61.41% | 45.75% | 28 | 50 |
| Scn10a_Sp_Exon_4_T1 | SCN10A | 4 | SpCas9 | 54.98% | 45.45% | 58 | 51 |
| Scn10a_Sp_Exon_12_T5 | SCN10A | 12 | SpCas9 | 61.45% | 45.41% | 27 | 52 |
| Scn10a_Sp_Exon_2_T8 | SCN10A | 2 | SpCas9 | 54.37% | 45.09% | 63 | 53 |
| Scn10a_Sp_Exon_11_T21 | SCN10A | 11 | SpCas9 | 55.63% | 44.13% | 54 | 54 |
| Scn10a_Sp_Exon_11_T17 | SCN10A | 11 | SpCas9 | 54.96% | 44.09% | 59 | 55 |
| Scn10a_Sp_Exon_11_T15 | SCN10A | 11 | SpCas9 | 48.65% | 44.08% | 95 | 56 |
| Scn10a_Sp_Exon_13_T19 | SCN10A | 13 | SpCas9 | 51.06% | 43.61% | 83 | 57 |
| Scn10a_Sp_Exon_6_T3 | SCN10A | 6 | SpCas9 | 54.41% | 43.58% | 62 | 58 |
| Scn10a_Sp_Exon_10_T1 | SCN10A | 10 | SpCas9 | 57.92% | 43.50% | 39 | 59 |
| Scn10a_Sp_Exon_11_T11 | SCN10A | 11 | SpCas9 | 55.69% | 43.45% | 53 | 60 |
| Scn10a_Sp_Exon_10_T6 | SCN10A | 10 | SpCas9 | 49.04% | 43.14% | 93 | 61 |
| Scn10a_Sp_Exon_12_T7 | SCN10A | 12 | SpCas9 | 52.96% | 43.13% | 71 | 62 |
| Scn10a_Sp_Exon_1_T5 | SCN10A | 1 | SpCas9 | 48.50% | 43.07% | 97 | 63 |
| Scn10a_Sp_Exon_13_T16 | SCN10A | 13 | SpCas9 | 50.90% | 42.78% | 86 | 64 |
| Scn10a_Sp_Exon_9_T13 | SCN10A | 9 | SpCas9 | 64.98% | 42.52% | 16 | 65 |
| Scn10a_Sp_Exon_6_T7 | SCN10A | 6 | SpCas9 | 58.51% | 42.38% | 37 | 66 |
| Scn10a_Sp_Exon_7_T4 | SCN10A | 7 | SpCas9 | 52.52% | 42.37% | 75 | 67 |
| Scn10a_Sp_Exon_9_T4 | SCN10A | 9 | SpCas9 | 53.88% | 42.24% | 66 | 68 |
| Scn10a_Sp_Exon_9_T9 | SCN10A | 9 | SpCas9 | 52.53% | 42.19% | 74 | 69 |
| Scn10a_Sp_Exon_10_T9 | SCN10A | 10 | SpCas9 | 47.84% | 42.05% | 100 | 70 |
| Scn10a_Sp_Exon_8_T2 | SCN10A | 8 | SpCas9 | 47.46% | 41.70% | 102 | 71 |
| Scn10a_Sp_Exon_1_T9 | SCN10A | 1 | SpCas9 | 50.32% | 41.44% | 90 | 72 |
| Scn10a_Sp_Exon_13_T15 | SCN10A | 13 | SpCas9 | 55.09% | 41.15% | 57 | 73 |
| Scn10a_Sp_Exon_1_T26 | SCN10A | 1 | SpCas9 | 48.17% | 40.83% | 99 | 74 |
| Scn10a_Sp_Exon_6_T8 | SCN10A | 6 | SpCas9 | 50.36% | 40.76% | 89 | 75 |
| Scn10a_Sp_Exon_1_T4 | SCN10A | 1 | SpCas9 | 51.44% | 40.43% | 80 | 76 |
| Scn10a_Sp_Exon_11_T22 | SCN10A | 11 | SpCas9 | 52.25% | 40.22% | 76 | 77 |
| Scn10a_Sp_Exon_1_T8 | SCN10A | 1 | SpCas9 | 50.94% | 40.09% | 85 | 78 |
| Scn10a_Sp_Exon_1_T28 | SCN10A | 1 | SpCas9 | 55.88% | 40.04% | 52 | 79 |
| Scn10a_Sp_Exon_1_T23 | SCN10A | 1 | SpCas9 | 48.87% | 39.94% | 94 | 80 |
| Scn10a_Sp_Exon_12_T14 | SCN10A | 12 | SpCas9 | 53.67% | 39.85% | 68 | 81 |
| Scn10a_Sp_Exon_10_T5 | SCN10A | 10 | SpCas9 | 45.47% | 39.07% | 104 | 82 |
| Scn10a_Sp_Exon_8_T9 | SCN10A | 8 | SpCas9 | 53.65% | 38.83% | 69 | 83 |
| Scn10a_Sp_Exon_10_T4 | SCN10A | 10 | SpCas9 | 53.90% | 38.71% | 65 | 84 |
| Scn10a_Sp_Exon_2_T6 | SCN10A | 2 | SpCas9 | 45.38% | 38.38% | 105 | 85 |
| Scn10a_Sp_Exon_8_T6 | SCN10A | 8 | SpCas9 | 57.30% | 38.26% | 43 | 86 |
| Scn10a_Sp_Exon_12_T9 | SCN10A | 12 | SpCas9 | 42.87% | 38.17% | 107 | 87 |
| Scn10a_Sp_Exon_2_T10 | SCN10A | 2 | SpCas9 | 47.78% | 37.61% | 101 | 88 |
| Scn10a_Sp_Exon_12_T1 | SCN10A | 12 | SpCas9 | 49.42% | 36.83% | 91 | 89 |
| Scn10a_Sp_Exon_14_T4 | SCN10A | 14 | SpCas9 | 61.35% | 35.92% | 29 | 90 |
| Scn10a_Sp_Exon_2_T9 | SCN10A | 2 | SpCas9 | 53.15% | 35.54% | 70 | 91 |
| Scn10a_Sp_Exon_1_T19 | SCN10A | 1 | SpCas9 | 48.57% | 35.36% | 96 | 92 |
| Scn10a_Sp_Exon_13_T19 | SCN10A | 11 | SpCas9 | 42.69% | 34.85% | 108 | 93 |
| Scn10a_Sp_Exon_11_T20 | SCN10A | 13 | SpCas9 | 39.77% | 34.85% | 120 | 94 |
| Scn10a_Sp_Exon_13_T10 | SCN10A | 13 | SpCas9 | 40.55% | 34.73% | 117 | 95 |
| Scn10a_Sp_Exon_12_T4 | SCN10A | 12 | SpCas9 | 42.32% | 34.32% | 111 | 96 |
| Scn10a_Sp_Exon_11_T29 | SCN10A | 11 | SpCas9 | 55.45% | 34.29% | 55 | 97 |
| Scn10a_Sp_Exon_1_T6 | SCN10A | 1 | SpCas9 | 41.41% | 33.80% | 114 | 98 |
| Scn10a_Sp_Exon_11_T5 | SCN10A | 11 | SpCas9 | 36.79% | 33.61% | 127 | 99 |
| Scn10a_Sp_Exon_8_T4 | SCN10A | 8 | SpCas9 | 48.33% | 33.60% | 98 | 100 |
| Scn10a_Sp_Exon_1_T20 | SCN10A | 1 | SpCas9 | 40.84% | 33.49% | 116 | 101 |
| Scn10a_Sp_Exon_11_T23 | SCN10A | 11 | SpCas9 | 52.78% | 33.30% | 73 | 102 |
| Scn10a_Sp_Exon_6_T4 | SCN10A | 6 | SpCas9 | 38.65% | 33.10% | 121 | 103 |
| Scn10a_Sp_Exon_1_T7 | SCN10A | 1 | SpCas9 | 44.90% | 32.82% | 106 | 104 |
| Scn10a_Sp_Exon_2_T2 | SCN10A | 2 | SpCas9 | 51.23% | 32.78% | 82 | 105 |
| Scn10a_Sp_Exon_1_T24 | SCN10A | 1 | SpCas9 | 42.10% | 32.67% | 112 | 106 |
| Scn10a_Sp_Exon_4_T5 | SCN10A | 4 | SpCas9 | 56.88% | 32.61% | 45 | 107 |
| Scn10a_Sp_Exon_9_T11 | SCN10A | 9 | SpCas9 | 52.09% | 32.18% | 79 | 108 |
| Scn10a_Sp_Exon_9_T1 | SCN10A | 9 | SpCas9 | 41.37% | 32.00% | 115 | 109 |
| Scn10a_Sp_Exon_11_T27 | SCN10A | 11 | SpCas9 | 55.20% | 31.85% | 56 | 110 |
| Scn10a_Sp_Exon_11_T1 | SCN10A | 11 | SpCas9 | 50.96% | 31.76% | 84 | 111 |
| Scn10a_Sp_Exon_11_T30 | SCN10A | 11 | SpCas9 | 34.52% | 31.72% | 133 | 112 |
| Scn10a_Sp_Exon_13_T1 | SCN10A | 13 | SpCas9 | 37.55% | 31.55% | 125 | 113 |
| Scn10a_Sp_Exon_9_T8 | SCN10A | 9 | SpCas9 | 52.96% | 31.47% | 72 | 114 |
| Scn10a_Sp_Exon_4_T3 | SCN10A | 4 | SpCas9 | 36.31% | 31.31% | 130 | 115 |
| Scn10a_Sp_Exon_1_T21 | SCN10A | 1 | SpCas9 | 42.58% | 31.22% | 109 | 116 |
| Scn10a_Sp_Exon_8_T5 | SCN10A | 8 | SpCas9 | 49.09% | 30.85% | 92 | 117 |
| Scn10a_Sp_Exon_13_T12 | SCN10A | 13 | SpCas9 | 42.38% | 30.40% | 110 | 118 |
| Scn10a_Sp_Exon_12_T13 | SCN10A | 12 | SpCas9 | 35.97% | 30.38% | 131 | 119 |
| Scn10a_Sp_Exon_6_T9 | SCN10A | 6 | SpCas9 | 34.43% | 30.28% | 134 | 120 |
| Scn10a_Sp_Exon_11_T4 | SCN10A | 11 | SpCas9 | 54.02% | 30.26% | 64 | 121 |
| Scn10a_Sp_Exon_11_T3 | SCN10A | 11 | SpCas9 | 38.32% | 30.19% | 123 | 122 |
| Scn10a_Sp_Exon_12_T10 | SCN10A | 12 | SpCas9 | 63.89% | 29.99% | 19 | 123 |
| Scn10a_Sp_Exon_7_T3 | SCN10A | 7 | SpCas9 | 38.44% | 29.43% | 122 | 124 |
| Scn10a_Sp_Exon_7_T5 | SCN10A | 7 | SpCas9 | 35.95% | 28.88% | 132 | 125 |
| Scn10a_Sp_Exon_8_T7 | SCN10A | 8 | SpCas9 | 40.06% | 28.67% | 118 | 126 |
| Scn10a_Sp_Exon_1_T11 | SCN10A | 1 | SpCas9 | 36.94% | 28.57% | 126 | 127 |
| Scn10a_Sp_Exon_11_T33 | SCN10A | 11 | SpCas9 | 36.65% | 27.54% | 129 | 128 |
| Scn10a_Sp_Exon_6_T10 | SCN10A | 6 | SpCas9 | 32.46% | 26.95% | 137 | 129 |
| Scn10a_Sp_Exon_11_T12 | SCN10A | 11 | SpCas9 | 32.19% | 26.77% | 138 | 130 |
| Scn10a_Sp_Exon_11_T7 | SCN10A | 11 | SpCas9 | 29.15% | 26.06% | 143 | 131 |
| Scn10a_Sp_Exon_1_T29 | SCN10A | 1 | SpCas9 | 46.08% | 25.89% | 103 | 132 |
| Scn10a_Sp_Exon_10_T13 | SCN10A | 10 | SpCas9 | 33.89% | 25.37% | 135 | 133 |
| Scn10a_Sp_Exon_13_T8 | SCN10A | 13 | SpCas9 | 37.57% | 25.01% | 124 | 134 |
| Scn10a_Sp_Exon_13_T11 | SCN10A | 13 | SpCas9 | 29.76% | 24.56% | 142 | 135 |
| Scn10a_Sp_Exon_5_T2 | SCN10A | 5 | SpCas9 | 31.74% | 24.55% | 139 | 136 |
| Scn10a_Sp_Exon_11_T13 | SCN10A | 11 | SpCas9 | 36.77% | 24.35% | 128 | 137 |
| Scn10a_Sp_Exon_5_T3 | SCN10A | 5 | SpCas9 | 30.72% | 23.38% | 140 | 138 |
| Scn10a_Sp_Exon_12_T12 | SCN10A | 12 | SpCas9 | 41.69% | 22.51% | 113 | 139 |
| Scn10a_Sp_Exon_9_T5 | SCN10A | 9 | SpCas9 | 56.19% | 22.49% | 49 | 140 |
| Scn10a_Sp_Exon_2_T1 | SCN10A | 2 | SpCas9 | 30.63% | 22.14% | 141 | 141 |
| Scn10a_Sp_Exon_11_T26 | SCN10A | 11 | SpCas9 | 28.87% | 22.10% | 144 | 142 |
| Scn10a_Sp_Exon_11_T8 | SCN10A | 11 | SpCas9 | 39.80% | 21.90% | 119 | 143 |
| Scn10a_Sp_Exon_11_T32 | SCN10A | 11 | SpCas9 | 26.86% | 20.23% | 148 | 144 |
| Scn10a_Sp_Exon_13_T14 | SCN10A | 13 | SpCas9 | 25.54% | 20.20% | 151 | 145 |
| Scn10a_Sp_Exon_1_T27 | SCN10A | 1 | SpCas9 | 23.64% | 20.09% | 155 | 146 |
| Scn10a_Sp_Exon_6_T2 | SCN10A | 6 | SpCas9 | 24.45% | 19.92% | 153 | 147 |
| Scn10a_Sp_Exon_13_T21 | SCN10A | 13 | SpCas9 | 26.89% | 19.88% | 147 | 148 |
| Scn10a_Sp_Exon_11_T2 | SCN10A | 11 | SpCas9 | 33.54% | 19.60% | 136 | 149 |
| Scn10a_Sp_Exon_11_T31 | SCN10A | 11 | SpCas9 | 22.87% | 18.35% | 156 | 150 |
| Scn10a_Sp_Exon_11_T20 | SCN10A | 11 | SpCas9 | 28.68% | 17.60% | 145 | 151 |
| Scn10a_Sp_Exon_1_T2 | SCN10A | 1 | SpCas9 | 21.35% | 17.50% | 158 | 152 |
| Scn10a_Sp_Exon_2_T4 | SCN10A | 2 | SpCas9 | 22.73% | 16.87% | 157 | 153 |
| Scn10a_Sp_Exon_14_T5 | SCN10A | 14 | SpCas9 | 26.01% | 16.71% | 150 | 154 |
| Scn10a_Sp_Exon_9_T10 | SCN10A | 9 | SpCas9 | 24.38% | 16.58% | 154 | 155 |
| Scn10a_Sp_Exon_11_T6 | SCN10A | 11 | SpCas9 | 17.51% | 13.60% | 160 | 156 |
| Scn10a_Sp_Exon_9_T6 | SCN10A | 9 | SpCas9 | 16.03% | 12.46% | 161 | 157 |
| Scn10a_Sp_Exon_1_T22 | SCN10A | 1 | SpCas9 | 25.18% | 12.18% | 152 | 158 |
| Scn10a_Sp_Exon_7_T1 | SCN10A | 7 | SpCas9 | 27.05% | 11.69% | 146 | 159 |
| Scn10a_Sp_Exon_12_T2 | SCN10A | 12 | SpCas9 | 26.39% | 11.56% | 149 | 160 |
| Scn10a_Sp_Exon_9_T12 | SCN10A | 9 | SpCas9 | 18.60% | 10.73% | 159 | 161 |
| Scn10a_Sp_Exon_13_T6 | SCN10A | 13 | SpCas9 | 9.77% | 7.31% | 163 | 162 |
| Scn10a_Sp_Exon_1_T12 | SCN10A | 1 | SpCas9 | 8.22% | 6.02% | 164 | 163 |
| Scn10a_Sp_Exon_9_T7 | SCN10A | 9 | SpCas9 | 12.54% | 5.44% | 162 | 164 |
| Scn10a_Sp_Exon_13_T22 | SCN10A | 13 | SpCas9 | 6.59% | 4.29% | 165 | 165 |
| Scn10a_Sp_Exon_11_T34 | SCN10A | 11 | SpCas9 | 3.63% | 2.42% | 166 | 166 |
| Scrambled control | N/A | N/A | SpCas9 | 0.27% | 0.23% | N/A | N/A |
| Untreated | N/A | N/A | N/A | 0.18% | 0.16% | N/A | N/A |

TABLE 11
Mean total indel percentage and mean frameshift-causing indel
percentages generated by SaCas9 gRNAs targeting SCN10A in iPSCs

| | | | | | Mean | | Rank |
|---|---|---|---|---|---|---|---|
| | | | | | frameshift- | Rank | based on |
| | | | | Mean total | causing | based on | frameshift- |
| | Target | Target | | indel | indel | total | causing |
| gRNA name | gene | exon | Cas9 | percentage | percentage | indels | indels |
| Scn10a_Sa_Exon_11_T1 | SCN10A | 11 | SaCas9 | 44.86% | 42.68% | 2 | 1 |
| Scn10a_Sa_Exon_1_T7 | SCN10A | 1 | SaCas9 | 42.84% | 40.36% | 4 | 2 |
| Scn10a_Sa_Exon_8_T5 | SCN10A | 8 | SaCas9 | 37.46% | 35.32% | 7 | 3 |
| Scn10a_Sa_Exon_12_T2 | SCN10A | 12 | SaCas9 | 43.31% | 32.36% | 3 | 4 |
| Scn10a_Sa_Exon_14_T1 | SCN10A | 14 | SaCas9 | 45.19% | 31.27% | 1 | 5 |
| Scn10a_Sa_Exon_14_T2 | SCN10A | 14 | SaCas9 | 39.18% | 27.17% | 5 | 6 |
| Scn10a_Sa_Exon_4_T5 | SCN10A | 4 | SaCas9 | 33.11% | 25.46% | 9 | 7 |
| Scn10a_Sa_Exon_1_T3 | SCN10A | 1 | SaCas9 | 28.02% | 24.67% | 13 | 8 |
| Scn10a_Sa_Exon_11_T2 | SCN10A | 11 | SaCas9 | 31.11% | 23.87% | 11 | 9 |
| Scn10a_Sa_Exon_11_T5 | SCN10A | 11 | SaCas9 | 29.04% | 23.03% | 12 | 10 |
| Scn10a_Sa_Exon_13_T9 | SCN10A | 13 | SaCas9 | 34.53% | 20.42% | 8 | 11 |
| Scn10a_Sa_Exon_3_T1 | SCN10A | 3 | SaCas9 | 27.38% | 19.18% | 14 | 12 |
| Scn10a_Sa_Exon_13_T2 | SCN10A | 13 | SaCas9 | 31.25% | 18.78% | 10 | 13 |
| Scn10a_Sa_Exon_10_T6 | SCN10A | 10 | SaCas9 | 20.74% | 17.76% | 20 | 14 |
| Scn10a_Sa_Exon_11_T10 | SCN10A | 11 | SaCas9 | 20.36% | 17.65% | 22 | 15 |
| Scn10a_Sa_Exon_2_T1 | SCN10A | 2 | SaCas9 | 19.59% | 17.00% | 27 | 16 |
| Scn10a_Sa_Exon_8_T1 | SCN10A | 8 | SaCas9 | 25.69% | 16.98% | 16 | 17 |
| Scn10a_Sa_Exon_4_T7 | SCN10A | 4 | SaCas9 | 20.06% | 16.79% | 24 | 18 |
| Scn10a_Sa_Exon_13_T4 | SCN10A | 13 | SaCas9 | 19.68% | 15.79% | 26 | 19 |
| Scn10a_Sa_Exon_11_T4 | SCN10A | 11 | SaCas9 | 20.35% | 15.76% | 23 | 20 |
| Scn10a_Sa_Exon_1_T6 | SCN10A | 1 | SaCas9 | 26.38% | 15.31% | 15 | 21 |
| Scn10a_Sa_Exon_7_T4 | SCN10A | 7 | SaCas9 | 22.50% | 15.28% | 17 | 22 |
| Scn10a_Sa_Exon_1_T8 | SCN10A | 1 | SaCas9 | 21.83% | 14.52% | 18 | 23 |
| Scn10a_Sa_Exon_11_T9 | SCN10A | 11 | SaCas9 | 38.57% | 13.91% | 6 | 24 |
| Scn10a_Sa_Exon_7_T3 | SCN10A | 7 | SaCas9 | 20.06% | 13.70% | 25 | 25 |
| Scn10a_Sa_Exon_11_T8 | SCN10A | 11 | SaCas9 | 21.79% | 13.40% | 19 | 26 |
| Scn10a_Sa_Exon_5_T2 | SCN10A | 5 | SaCas9 | 16.95% | 13.18% | 29 | 27 |
| Scn10a_Sa_Exon_1_T4 | SCN10A | 1 | SaCas9 | 18.11% | 13.04% | 28 | 28 |
| Scn10a_Sa_Exon_6_T6 | SCN10A | 6 | SaCas9 | 20.70% | 10.65% | 21 | 29 |
| Scn10a_Sa_Exon_1_T5 | SCN10A | 1 | SaCas9 | 14.58% | 10.63% | 32 | 30 |
| Scn10a_Sa_Exon_4_T2 | SCN10A | 4 | SaCas9 | 13.82% | 10.55% | 33 | 31 |
| Scn10a_Sa_Exon_10_T2 | SCN10A | 10 | SaCas9 | 15.04% | 9.94% | 31 | 32 |
| Scn10a_Sa_Exon_10_T7 | SCN10A | 10 | SaCas9 | 16.61% | 8.83% | 30 | 33 |
| Scn10a_Sa_Exon_13_T3 | SCN10A | 13 | SaCas9 | 12.36% | 8.21% | 35 | 34 |
| Scn10a_Sa_Exon_10_T5 | SCN10A | 10 | SaCas9 | 10.62% | 7.87% | 37 | 35 |
| Scn10a_Sa_Exon_10_T3 | SCN10A | 10 | SaCas9 | 13.45% | 7.63% | 34 | 36 |
| Scn10a_Sa_Exon_8_T4 | SCN10A | 8 | SaCas9 | 7.75% | 6.06% | 41 | 37 |
| Scn10a_Sa_Exon_11_T7 | SCN10A | 11 | SaCas9 | 6.42% | 5.61% | 46 | 38 |
| Scn10a_Sa_Exon_7_T2 | SCN10A | 7 | SaCas9 | 7.54% | 5.53% | 42 | 39 |
| Scn10a_Sa_Exon_13_T5 | SCN10A | 13 | SaCas9 | 5.84% | 5.42% | 48 | 40 |
| Scn10a_Sa_Exon_13_T8 | SCN10A | 13 | SaCas9 | 7.45% | 5.16% | 43 | 41 |
| Scn10a_Sa_Exon_3_T2 | SCN10A | 3 | SaCas9 | 6.18% | 5.14% | 47 | 42 |
| Scn10a_Sa_Exon_4_T4 | SCN10A | 4 | SaCas9 | 10.64% | 4.88% | 36 | 43 |
| Scn10a_Sa_Exon_6_T3 | SCN10A | 6 | SaCas9 | 8.43% | 4.83% | 39 | 44 |
| Scn10a_Sa_Exon_10_T8 | SCN10A | 10 | SaCas9 | 6.74% | 4.64% | 44 | 45 |
| Scn10a_Sa_Exon_1_T9 | SCN10A | 1 | SaCas9 | 5.42% | 4.53% | 49 | 46 |
| Scn10a_Sa_Exon_13_T1 | SCN10A | 13 | SaCas9 | 6.56% | 4.34% | 45 | 47 |
| Scn10a_Sa_Exon_6_T8 | SCN10A | 6 | SaCas9 | 8.25% | 3.89% | 40 | 48 |
| Scn10a_Sa_Exon_4_T3 | SCN10A | 4 | SaCas9 | 4.37% | 3.72% | 51 | 49 |
| Scn10a_Sa_Exon_5_T4 | SCN10A | 5 | SaCas9 | 5.02% | 3.69% | 50 | 50 |
| Scn10a_Sa_Exon_6_T5 | SCN10A | 6 | SaCas9 | 4.27% | 3.66% | 52 | 51 |
| Scn10a_Sa_Exon_13_T7 | SCN10A | 13 | SaCas9 | 9.36% | 2.84% | 38 | 52 |
| Scn10a_Sa_Exon_10_T4 | SCN10A | 10 | SaCas9 | 2.67% | 2.40% | 54 | 53 |
| Scn10a_Sa_Exon_5_T1 | SCN10A | 5 | SaCas9 | 2.76% | 2.36% | 53 | 54 |
| Scn10a_Sa_Exon_2_T2 | SCN10A | 2 | SaCas9 | 2.29% | 1.87% | 55 | 55 |
| Scn10a_Sa_Exon_14_T3 | SCN10A | 14 | SaCas9 | 2.16% | 1.62% | 57 | 56 |
| Scn10a_Sa_Exon_3_T3 | SCN10A | 3 | SaCas9 | 2.17% | 1.53% | 56 | 57 |
| Scn10a_Sa_Exon_12_T4 | SCN10A | 12 | SaCas9 | 2.07% | 1.52% | 59 | 58 |
| Scn10a_Sa_Exon_7_T5 | SCN10A | 7 | SaCas9 | 2.12% | 1.42% | 58 | 59 |
| Scn10a_Sa_Exon_6_T1 | SCN10A | 6 | SaCas9 | 1.69% | 1.21% | 61 | 60 |
| Scn10a_Sa_Exon_11_T3 | SCN10A | 11 | SaCas9 | 1.93% | 0.77% | 60 | 61 |
| Scn10a_Sa_Exon_6_T4 | SCN10A | 6 | SaCas9 | 0.73% | 0.53% | 64 | 62 |
| Scn10a_Sa_Exon_9_T1 | SCN10A | 9 | SaCas9 | 0.79% | 0.47% | 63 | 63 |
| Scn10a_Sa_Exon_10_T1 | SCN10A | 10 | SaCas9 | 0.46% | 0.45% | 65 | 64 |
| Scn10a_Sa_Exon_14_T4 | SCN10A | 14 | SaCas9 | 1.24% | 0.33% | 62 | 65 |
| Scn10a_Sa_Exon_9_T3 | SCN10A | 9 | SaCas9 | 0.28% | 0.20% | 67 | 66 |
| Scn10a_Sa_Exon_1_T1 | SCN10A | 1 | SaCas9 | 0.20% | 0.17% | 68 | 67 |
| Scn10a_Sa_Exon_13_T6 | SCN10A | 13 | SaCas9 | 0.35% | 0.17% | 66 | 68 |
| Scn10a_Sa_Exon_12_T1 | SCN10A | 12 | SaCas9 | 0.18% | 0.16% | 69 | 69 |
| Scn10a_Sa_Exon_8_T2 | SCN10A | 8 | SaCas9 | 0.11% | 0.10% | 70 | 70 |
| Scn10a_Sa_Exon_12_T5 | SCN10A | 12 | SaCas9 | 0.06% | 0.06% | 71 | 71 |
| Scn10a_Sa_Exon_8_T3 | SCN10A | 8 | SaCas9 | 0.03% | 0.03% | 72 | 72 |
| Scn10a_Sa_Exon_6_T7 | SCN10A | 6 | SaCas9 | 0.00% | 0.00% | 73 | 73 |
| Scrambled control | N/A | N/A | SaCas9 | 0.27% | 0.23% | N/A | N/A |
| Untreated | N/A | N/A | N/A | 0.18% | 0.14% | N/A | N/A |

Screening of Top Ranked gRNAs Targeted to SCN9A and SCN10A in iPSCs Stably Expressing SpCas9 or SaCas9, and in iPSC-Derived Sensory Neurons

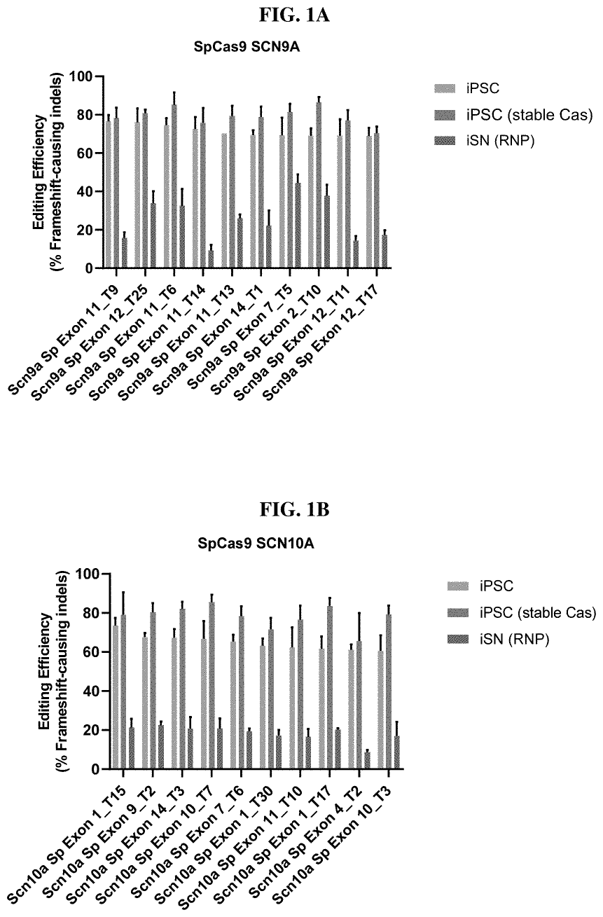

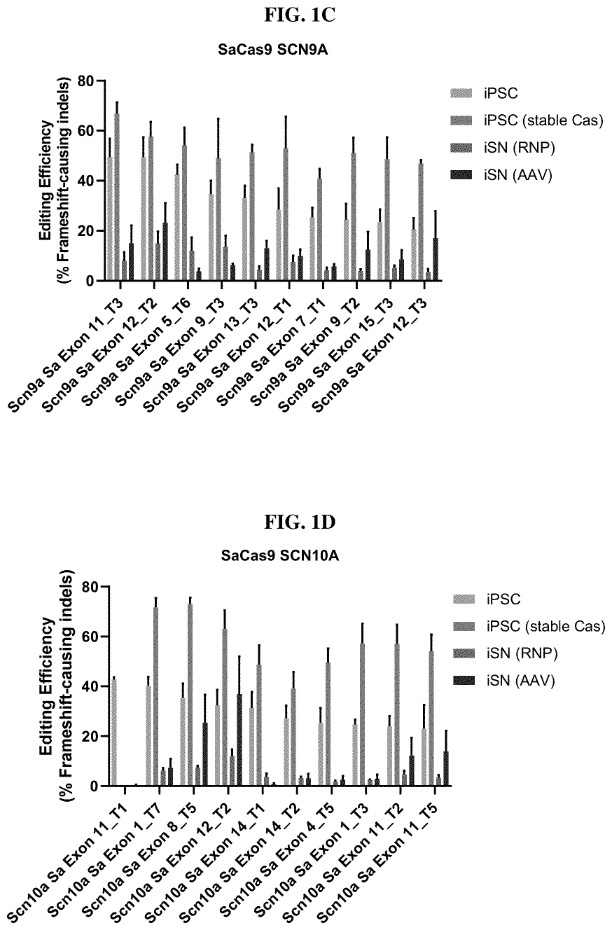

Based on on-target efficacy in initial gRNA screens in iPSCs, 40 guides were prioritized for further on-target editing studies in additional cell models such as iPSCs stably expressing Cas9 and iPSC-derived sensory neurons (iSNs) (FIGS. 1A-1D). Specifically, ten guides from each of four categories were chosen: 1) ten gRNAs for SpCas9 targeting SCN9A, 2) ten gRNAs for SpCas9 targeting SCN10a, 3) ten gRNAs for SaCas9 targeting SCN9A, and 4) ten gRNAs for SaCas9 targeting SCN10a (Tables 12 and 13).

These 40 prioritized gRNAs were screened in engineered iPSCs stably expressing either SpCas9 or SaCas9. Synthetic gRNAs were electroporated into the corresponding cell line. These 40 gRNAs were already screened for on-target editing efficiency in iSNs. In iSNs, RNP complexes were electroporated into the adherent neuronal cultures for all 40 gRNAs. In addition, the 20 SaCas9 gRNAs were also delivered to iSNs by all-in-one AAV vectors expressing SaCas9 and a gRNA. Genomic DNA was purified from treated cells for sequencing analysis as described in the methods.

In each model, two independent experiments were conducted. The mean average cutting efficiency was calculated based on four replicates for each sample looking at total percentage of insertions and deletions (indels) at the predicted cut site of each gRNA. For the purposes of knocking out the SCN9A and SCN10A genes, the average percentage of indels that would result in a frameshift mutation was also calculated. Guide RNAs were ranked on mean frameshift-causing indel percentage. A summary of the on-target editing efficiencies of these 40 prioritized gRNAs across different cell models can be found in FIGS. 1A-1D, as well as in Tables 12 and 13.

TABLE 12
Summary of on-target editing efficiency of prioritized SpCas9
gRNAs across different cells models

| | | | % Frameshift Indels | | | | | |
|---|---|---|---|---|---|---|---|---|
| | SEQ | | | stable | iSNs | | iPSC | |
| | ID | | | Cas9 | (RNP | iPSC | stable | iSN |
| gRNA Name | NO: | Sequence | iPSCs | iPSCs | electroporation) | rank | rank | rank |

SCN9A SpCas9

| | | | | | | | | |
|---|---|---|---|---|---|---|---|---|
| Scn9a Sp Exon 11_T9 | 1 | CAAuuuGGGuGGuACCuGAu | 76.55% | 78.18% | 15.83% | 1 | 7 | 8 |
| Scn9a Sp Exon 12_T25 | 2 | GCuuCGCCuuGCAGAAAACA | 76.05% | 80.74% | 33.90% | 2 | 4 | 3 |
| Scn9a Sp Exon 11_T6 | 3 | GCCuAuGCCCuuCGACACCA | 74.56% | 85.16% | 32.52% | 3 | 2 | 4 |
| Scn9a Sp Exon 11_T14 | 4 | AuAGGCGAGCACAuGAAAAG | 72.51% | 75.68% | 9.20% | 4 | 9 | 10 |
| Scn9a Sp Exon 11_T13 | 5 | CGGCuGAAuAuACAAGuAuu | 70.14% | 79.25% | 26.04% | 5 | 5 | 5 |
| Scn9a Sp Exon 14_T1 | 6 | GGAACACCACCCAAuGACuG | 69.46% | 78.77% | 22.14% | 6 | 6 | 6 |
| Scn9a Sp Exon 7_T5 | 7 | CAGGCCuGAAGACAAuuGuA | 69.19% | 81.38% | 44.42% | 7 | 3 | 1 |
| Scn9a Sp Exon 2_T10 | 8 | GGAAuGuCCCCAuAGAuGAA | 69.08% | 86.34% | 37.78% | 8 | 1 | 2 |
| Scn9a Sp Exon 12_T11 | 9 | CCACCAAuGCuGCCGGuGAA | 69.01% | 77.06% | 14.40% | 9 | 8 | 9 |
| Scn9a Sp Exon 12_T17 | 10 | CAGuCACCACuCAGCAuuCG | 68.85% | 70.36% | 17.25% | 10 | 10 | 7 |

SCN10A SpCas9

| | | | | | | | | |
|---|---|---|---|---|---|---|---|---|
| Scn10a Sp Exon 1_T15 | 21 | GCuCCCCGAuCAGuuCuGCu | 73.46% | 79.12% | 21.33% | 1 | 6 | 2 |
| Scn10a Sp Exon 9_T2 | 22 | uGuAGuCACCAuGGCGuAuG | 67.64% | 80.40% | 22.50% | 2 | 4 | 1 |
| Scn10a Sp Exon 14_T3 | 23 | GGAAGCuCCGCAGCACAGAC | 67.26% | 82.00% | 20.91% | 3 | 3 | 3 |
| Scn10a Sp Exon 10_T7 | 24 | uCCuuACAACCAGCGCAGGA | 66.79% | 85.44% | 20.77% | 4 | 1 | 4 |
| Scn10a Sp Exon 7_T6 | 25 | ACuuCuGACCCCuuACuGuG | 65.31% | 78.28% | 19.31% | 5 | 7 | 6 |
| Scn10a Sp Exon 1_T30 | 26 | GAGCuCCCAGCAGAACuGAu | 63.21% | 71.66% | 17.33% | 6 | 9 | 7 |
| Scn10a Sp Exon 11_T10 | 27 | CCGAGACAuCGACAGCuCCA | 62.38% | 76.53% | 16.60% | 7 | 8 | 9 |
| Scn10a Sp Exon 1_T17 | 28 | AuCCGuuCuACAGCACACAC | 61.62% | 83.60% | 20.25% | 8 | 2 | 5 |
| Scn10a Sp Exon 4_T2 | 29 | uCACGuACCuGAGAGAuCCu | 61.10% | 65.50% | 8.80% | 9 | 10 | 10 |
| Scn10a Sp Exon 10_T3 | 30 | CGCAGGuGCuAGCAGCACuA | 60.57% | 79.22% | 17.04% | 10 | 5 | 8 |

TABLE 13
Summary of on-target editing efficiency of prioritized SaCas9 gRNAs
across different cells models

| | | | % Frameshift Indels | | | | | | | |
|---|---|---|---|---|---|---|---|---|---|---|
| | | | | | iSNs | iSNs | | | | |
| | SEQ | | | stable | (RNP | (AAV | | iPSC | iSN | iSN |
| | ID | | | Cas9 | electro- | trans- | iPSC | stable | (RNP) | (AAV) |
| gRNA Name | NO: | Sequence | iPSCs | iPSCs | poration) | duction) | rank | rank | rank | rank |

SCN9A SaCas9

| | | | | | | | | | | |
|---|---|---|---|---|---|---|---|---|---|---|
| Scn9a Sa Exon 11_T3 | 11 | AAGCAGAAuuAuGGGCCuCuCA | 49.49% | 66.77% | 7.87% | 14.87% | 1 | 1 | 4 | 3 |
| Scn9a Sa Exon 12_T2 | 12 | GCCuuGCAGAAAACAAGGAGCC | 49.44% | 57.62% | 14.87% | 23.22% | 2 | 2 | 1 | 1 |
| Scn9a Sa Exon 5_T6 | 13 | ACGACAAAAuCCAGCCAGuuCC | 42.37% | 54.16% | 11.97% | 3.79% | 3 | 3 | 3 | 10 |
| Scn9a Sa Exon 9_T3 | 14 | CuGGGAAAACCuuuACCAACAG | 34.60% | 48.90% | 13.50% | 6.20% | 4 | 7 | 2 | 8 |
| Scn9a Sa Exon 13_T3 | 15 | uCCCAACCuCAGACAGAGAGCA | 33.08% | 51.37% | 4.33% | 13.04% | 5 | 5 | 7 | 4 |
| Scn9a Sa Exon 12_T1 | 16 | GAuGuuACuGCuGCGuCGCuCC | 28.42% | 52.96% | 7.57% | 9.96% | 6 | 4 | 5 | 6 |
| Scn9a Sa Exon 7_T1 | 17 | CAuGAuCCuGACuGuGuuCuGu | 25.39% | 40.83% | 4.22% | 5.70% | 7 | 10 | 8 | 9 |
| Scn9a Sa Exon 9_T2 | 18 | CuCGuGuGuAGuCAGuGuCCAG | 24.43% | 51.04% | 3.84% | 12.52% | 8 | 6 | 9 | 5 |
| Scn9a Sa Exon 15_T3 | 19 | AAACuGAuuGCCAuGGAuCCAu | 23.51% | 48.59% | 5.07% | 8.54% | 9 | 8 | 6 | 7 |
| Scn9a Sa Exon 12_T3 | 20 | AGAAAACAAGGAGCCACGAAuG | 20.49% | 46.80% | 3.51% | 17.10% | 10 | 9 | 10 | 2 |

SCN10A SaCas9

| | | | | | | | | | | |
|---|---|---|---|---|---|---|---|---|---|---|
| Scn10a Sa Exon 11_T1 | 31 | CCCuGGAGCuGuCGAuGuCuCG | 42.68% | 0.00% | 0.00% | 0.39% | 1 | 10 | 10 | 10 |
| Scn10a Sa Exon 1_T7 | 32 | uAGAuCCGuuCuACAGCACACA | 40.36% | 71.64% | 6.10% | 7.31% | 2 | 2 | 3 | 5 |
| Scn10a Sa Exon 8_T5 | 33 | AGuGAGAGGAAAGCCCAAGCAA | 35.32% | 73.07% | 7.37% | 25.44% | 3 | 1 | 2 | 2 |
| Scn10a Sa Exon 12_T2 | 34 | ACCuuuCCGGGCCCAAAGGGCA | 32.36% | 63.10% | 11.88% | 36.91% | 4 | 3 | 1 | 1 |
| Scn10a Sa Exon 14_T1 | 35 | CuuuGACuGCAuCAuCGuCACu | 31.27% | 48.68% | 3.67% | 0.72% | 5 | 8 | 5 | 9 |
| Scn10a Sa Exon 14_T2 | 36 | CACuuCuuCuGGAAAuAAuAGu | 27.17% | 39.07% | 3.16% | 3.03% | 6 | 9 | 7 | 6 |
| Scn10a Sa Exon 4_T5 | 37 | AuuuuAGCGuCAuuACCCuGGC | 25.46% | 49.54% | 1.86% | 2.52% | 7 | 7 | 9 | 8 |
| Scn10a Sa Exon 1_T3 | 38 | AACAACuuCCGuCGCuuuACuC | 24.67% | 57.17% | 2.36% | 2.87% | 8 | 4 | 8 | 7 |
| Scn10a Sa Exon 11_T2 | 39 | GCCGAGAuAuCuCACuCCCuGA | 23.87% | 56.97% | 4.61% | 12.23% | 9 | 5 | 4 | 4 |
| Scn10a Sa Exon 11_T5 | 40 | uGGuGuuCAuCuuCuCCAuGCC | 23.03% | 54.02% | 3.32% | 13.89% | 10 | 6 | 6 | 3 |

Off-Target Evaluation of SpCas9 and SaCas9 gRNAs Targeted to SCN9A and SCN10A in iPSCs

Based on on-target efficacy in initial gRNA screens in iPSCs, 40 guides were also prioritized for an off-target evaluation. Specifically, ten guides from each of four categories were chosen: 1) ten gRNAs for SpCas9 targeting SCN9A, 2) ten gRNAs for SpCas9 targeting SCN10a, 3) ten gRNAs for SaCas9 targeting SCN9A, and 4) ten gRNAs for SaCas9 targeting SCN10a.

Of the 40 gRNAs included in the study, 29 gRNAs were categorized as “Tier 1” (Table 14), where no off-target sites included in the study entered statistical testing; these 29 gRNAs included 4 gRNAs where no off-target sites were predicted under the sequence similarity criteria. Based on this study, these 29 gRNAs are considered to have no evidence of off-target editing. In addition, seven gRNAs were categorized as “Tier 2” (Table 15), where at least one off-target site associated with that gRNAs may have entered statistical testing, but was not found to be statistically significant. These off-target profile of these gRNAs are considered to be inconclusive from this study. In addition, four gRNAs were categorized as “Tier 3” (Table 16), where at least one off-target site was found to have statistically significant off-target editing. These gRNAs were strongly deprioritized, based on these off-target editing results. All combinations of target genes (SCN9A or SCN10A) and enzymes (SpCas9 or SaCas9) were found to have at least 5 Tier 1 guides.

TABLE 14
29 gRNAs categorized as Tier 1 with
no evidence of off-target editing

| | | | # sites |
|---|---|---|---|
| | | | with any |
| | SEQ | # tested | evidence of |
| gRNA Name | ID NO: | sites | editing |
| Scn9a Sp Exon 11_T9 | 1 | 44 | 0 |
| Scn9a Sp Exon 11_T6 | 3 | 42 | 0 |
| Scn9a Sp Exon 11_T14 | 4 | 83 | 0 |
| Scn9a Sp Exon 11_T13 | 5 | 40 | 0 |
| Scn9a Sp Exon 12_T11 | 9 | 42 | 0 |
| Scn10a Sp Exon 10_T3 | 30 | 42 | 0 |
| Scn10a Sp Exon 9_T2 | 22 | 54 | 0 |
| Scn10a Sp Exon 14_T3 | 23 | 104 | 0 |
| Scn10a Sp Exon 7_T6 | 25 | 117 | 0 |
| Scn9a Sa Exon 12_T3 | 20 | 1 | 0 |
| Scn9a Sa Exon 12_T1 | 16 | 1 | 0 |
| Scn9a Sa Exon 12_T2 | 12 | 2 | 0 |
| Scn9a Sa Exon 5_T6 | 13 | 1 | 0 |
| Scn9a Sa Exon 9_T3 | 14 | 1 | 0 |
| Scn9a Sa Exon 13_T3 | 15 | 2 | 0 |
| Scn9a Sa Exon 11_T3 | 11 | 2 | 0 |
| Scn9a Sa Exon 9_T2 | 18 | 1 | 0 |
| Scn9a Sa Exon 15_T3 | 19 | 2 | 0 |
| Scn10a Sa Exon 11_T5 | 40 | 2 | 0 |
| Scn10a Sa Exon 8_T5 | 33 | 4 | 0 |
| Scn10a Sa Exon 14_T1 | 35 | 2 | 0 |
| Scn10a Sa Exon 14_T2 | 36 | 4 | 0 |
| Scn10a Sa Exon 4_T5 | 37 | 1 | 0 |
| Scn10a Sa Exon 11_T2 | 39 | 1 | 0 |
| Scn10a Sp Exon 10_T7 | 24 | 68 | 1* |
| Scn10a Sa Exon 11_T1 | 31 | 0 | 0 |
| Scn10a Sa Exon 1_T7 | 32 | 0 | 0 |
| Scn10a Sa Exon 12_T2 | 34 | 0 | 0 |
| Scn10a Sa Exon 1_T3 | 38 | 0 | 0 |

*Scn10a Sp Exon 10_T7 had one site tested due to the 0.2% threshold requirement, but that site was ultimately excluded due to a germline mutation.

TABLE 15
Seven gRNAs categorized as Tier 2 with inconclusive off-target editing profiles

| | SEQ | | T-test | Chi-sq | Treated | Untreated | # | # |
|---|---|---|---|---|---|---|---|---|
| gRNA name | ID NO: | Site ID | pval | pval | (Indel %) | (Indel %) | mismatch | gaps |
| Scn10a Sp Exon | 27 | chr8: 3193432- | 0.0981 | 0 | 0.89% | 0.00% | 2 | 1 |
| 11_T10 | | 3193453 | | | | | | |
| Scn9a Sa Exon | 17 | chr2: 165162747- | 0.0715 | 0 | 0.67% | 0.00% | 1 | 1 |
| 7_T1 | | 165162773 | | | | | | |
| Scn9a Sp Exon | 7 | chr12: 51699567- | 0.2293 | 0 | 0.62% | 0.00% | 0 | 1 |
| 7_T5 | | 51699588 | | | | | | |
| Scn9a Sp Exon | 7 | chr17: 49809267- | 0.1707 | 0.0006 | 0.35% | 0.00% | 2 | 0 |
| 7_T5 | | 49809289 | | | | | | |
| Scn9a Sp Exon | 2 | chr5: 139982158- | 0.0769 | 0 | 0.53% | 0.00% | 3 | 0 |
| 12_T25 | | 139982180 | | | | | | |
| Scn9a Sp Exon | 6 | chr18: 34382228- | 0.0929 | 0.0140 | 0.17% | 0.00% | 3 | 0 |
| 14_T1 | | 34382250 | | | | | | |
| Scn9a Sp Exon | 10 | chr4: 41330183- | 0.0841 | 0.0114 | 0.16% | 0.00% | 2 | 0 |
| 12_T17 | | 41330205 | | | | | | |
| Scn10a Sp Exon | 29 | chr1: 191298075- | 0.1989 | 0.0107 | 0.15% | 0.00% | 3 | 0 |
| 4_T2 | | 191298097 | | | | | | |

TABLE 16
Four gRNAs categorized as Tier 3 with off-target editing confirmed (p < .05) at one or more sites

| | SEQ | | T-test | Chi-sq | Treated | Untreated | # | # |
|---|---|---|---|---|---|---|---|---|
| gRNA name | ID NO: | Site ID | pval | pval | (Indel %) | (Indel %) | mismatch | gaps |
| Scn9a Sp Exon | 8 | chr12: 51663013- | 0.020 | 3.52E−18 | 1.80% | 0.00% | 2 | 0 |
| 2_T10 | | 51663035 | | | | | | |
| Scn9a Sp Exon | 8 | chr4: 64749297- | 0.030 | 6.26E−116 | 8.50% | 0.00% | 3 | 0 |
| 2_T10 | | 64749319 | | | | | | |
| Scn10a Sp Exon | 21 | chr15: 31179489- | 0.050 | 1.74E−13 | 3.90% | 0.00% | 3 | 0 |
| 1_T15 | | 31179511 | | | | | | |
| Scn10a Sp Exon | 21 | chr16: 89566197- | 0.080 | 3.48E−07 | 0.70% | 0.00% | 2 | 1 |
| 1_T15 | | 89566218 | | | | | | |
| Scn10a Sp Exon | 26 | chr2: 180785404- | 0.010 | 9.20E−07 | 3.20% | 0.00% | 3 | 0 |
| 1_T30 | | 180785426 | | | | | | |
| Scn10a Sp Exon | 26 | chr5: 111925220- | 0.090 | 7.59E−09 | 0.60% | 0.00% | 2 | 1 |
| 1_T30 | | 111925241 | | | | | | |
| Scn10a Sp Exon | 28 | chr1: 17434745- | 0.000 | 7.48E−75 | 6.10% | 0.00% | 2 | 0 |
| 1_T17 | | 17434767 | | | | | | |
| Scn10a Sp Exon | 28 | chr9: 134428917- | 0.030 | 0.000920453 | 0.40% | 0.00% | 3 | 0 |
| 1_T17 | | 134428939 | | | | | | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A gene editing system for modifying a sodium voltage-gated channel alpha subunit 9 (SCN9A) gene, the gene editing system comprising:
(a) an RNA-guided DNA endonuclease or a first polynucleotide moiety, which comprises a first nucleotide sequence encoding the RNA-guided DNA endonuclease; and
(b) a second polynucleotide moiety, which comprises a second nucleotide sequence encoding a guide RNA (gRNA), wherein the gRNA comprises a spacer sequence having the nucleotide sequence of any one of SEQ ID NOs: 1-20.

2. The gene editing system of claim 1, wherein: (i) the RNA-guided DNA endonuclease of (a) is Staphylococcus pyogenes Cas9 (SpCas9); and (ii) the gRNA of (b) comprises a spacer sequence having the nucleotide sequence of any one of SEQ ID NOs: 1-10.

3. (canceled)

4. The gene editing system of claim 1, wherein: (i) the RNA-guided DNA endonuclease of (a) is Staphylococcus aureus Cas9 (SaCas9); and (ii) the gRNA of (b) comprises a spacer sequence having the nucleotide sequence of any one of SEQ ID NOs: 11-20.

5. (canceled)

6. The gene editing system of claim 1, wherein the gRNA of (b) further comprises a scaffold sequence, optionally wherein the scaffold sequence comprises the nucleotide sequence of SEQ ID NO: 41.

7. (canceled)

8. The gene editing system of claim 1, wherein the first nucleotide sequence encoding the RNA-guided endonuclease in (a) further comprises a nucleotide sequence encoding a nuclear localization signal (NLS), which is fused in-frame with the RNA-guided DNA endonuclease.

9. (canceled)

10. The gene editing system of claim 1, wherein the first polynucleotide moiety of (a) and the second polynucleotide moiety of (b) are of different polynucleotides, optionally wherein at least one of the different polynucleotides is a viral vector.

11.-12. (canceled)

13. The gene editing system of claim 1, wherein a single polynucleotide comprises the first polynucleotide moiety of (a) and the second polynucleotide moiety of (b), optionally wherein the single polynucleotide is a viral vector.

14.-15. (canceled)

16. A nucleic acid comprising the single polynucleotide of claim 13.

17. A viral particle or a set of viral particles, which collectively comprises the gene editing system of claim 1.

18. (canceled)

19. A method of editing a sodium voltage-gated channel alpha subunit 9 (SCN9A) gene, the method comprising contacting a cell with:
a gene editing system of claim 1.

20. The method of claim 19, wherein the contacting step is performed by administering the gene editing system to a subject in need thereof.

21.-26. (canceled)

27. The method of claim 19, further comprising administering the cell to a subject in need thereof, optionally wherein the cell is a stem cell.

28. (canceled)

29. A gene editing system for modifying a sodium voltage-gated channel alpha subunit 10 (SCNA10) gene, the gene editing system comprising:
(a) an RNA-guided DNA endonuclease or a first polynucleotide moiety, which comprises a first nucleotide sequence encoding the RNA-guided DNA endonuclease; and
(b) a second polynucleotide moiety, which comprises a second nucleotide sequence encoding a guide RNA (gRNA), wherein the gRNA comprises a spacer sequence having the nucleotide sequence of any one of SEQ ID NOs: 21-40.

30. The gene editing system of claim 29, wherein: (i) the RNA-guided DNA endonuclease of (a) is Staphylococcus pyogenes Cas9 (SpCas9); and (ii) the gRNA of (b) comprises a spacer sequence having the nucleotide sequence of any one of SEQ ID NOs: 21-30.

31. (canceled)

32. The gene editing system of claim 29, wherein: (i) the RNA-guided DNA endonuclease of (a) is Staphylococcus aureus Cas9 (SaCas9); and (iii) the gRNA of (b) comprises a spacer sequence having the nucleotide sequence of any one of SEQ ID NOs: 31-40.

33. The gene editing system of claim 29, wherein the gRNA of (b) further comprises a scaffold sequence, optionally wherein the scaffold sequence comprises the nucleotide sequence of SEQ ID NO: 41.

34. (canceled)

35. The gene editing system of claim 29, wherein the first nucleotide sequence encoding the RNA-guided endonuclease in (a) further comprises a nucleotide sequence encoding a nuclear localization signal (NLS), which is fused in-frame with the RNA-guided DNA endonuclease.

36. (canceled)

37. The gene editing system of claim 29, wherein the first polynucleotide moiety of (a) and the second polynucleotide moiety of (b) are of different polynucleotides, optionally wherein at least one of the different polynucleotides is a viral vector.

38.-39. (canceled)

40. The gene editing system of claim 29, wherein a single polynucleotide comprises the first polynucleotide moiety of (a) and the second polynucleotide moiety of (b), optionally wherein the single polynucleotide is a viral vector.

41.-42. (canceled)

43. A nucleic acid comprising the single polynucleotide of claim 40.

44. A viral particle or a set of viral particles, which collectively comprises the gene editing system of claim 29.

45. (canceled)

46. A method of editing a sodium voltage-gated channel alpha subunit 10 (SCNA10) gene, the method comprising contacting a cell with:
a gene editing system of claim 29.

47. The method of claim 46, wherein the contacting step is performed by administering the gene editing system to a subject in need thereof.

48.-53. (canceled)

54. The method of claim 46, further comprising administering the cell to a subject in need thereof, optionally wherein the cell is a stem cell.

55. (canceled)

56. A method of treating a subject having pain, the method comprising administering to the subject
the gene-editing system of claim 1.

57. (canceled)

58. A method of treating a subject having pain, the method comprising administering to the subject the gene-editing system of claim 29.


SEQUENCE LISTING (499 sequences)

SEQ ID NO: 1 - RNA, 20 nt, Artificial Sequence
  Synthetic Polynucleotide
caauuugggugguaccugau

SEQ ID NO: 2 - RNA, 20 nt, Artificial Sequence
  Synthetic Polynucleotide
gcuucgccuugcagaaaaca

SEQ ID NO: 3 - RNA, 20 nt, Artificial Sequence
  Synthetic Polynucleotide
gccuaugcccuucgacacca

SEQ ID NO: 4 - RNA, 20 nt, Artificial Sequence
  Synthetic Polynucleotide
auaggcgagcacaugaaaag

SEQ ID NO: 5 - RNA, 20 nt, Artificial Sequence
  Synthetic Polynucleotide
cggcugaauauacaaguauu

SEQ ID NO: 6 - RNA, 20 nt, Artificial Sequence
  Synthetic Polynucleotide
ggaacaccacccaaugacug

SEQ ID NO: 7 - RNA, 20 nt, Artificial Sequence
  Synthetic Polynucleotide
caggccugaagacaauugua

SEQ ID NO: 8 - RNA, 20 nt, Artificial Sequence
  Synthetic Polynucleotide
ggaauguccccauagaugaa

SEQ ID NO: 9 - RNA, 20 nt, Artificial Sequence
  Synthetic Polynucleotide
ccaccaaugcugccggugaa

SEQ ID NO: 10 - RNA, 20 nt, Artificial Sequence
  Synthetic Polynucleotide
cagucaccacucagcauucg

SEQ ID NO: 11 - RNA, 22 nt, Artificial Sequence
  Synthetic Polynucleotide
aagcagaauuaugggccucuca

SEQ ID NO: 12 - RNA, 22 nt, Artificial Sequence
  Synthetic Polynucleotide
gccuugcagaaaacaaggagcc

SEQ ID NO: 13 - RNA, 22 nt, Artificial Sequence
  Synthetic Polynucleotide
acgacaaaauccagccaguucc

SEQ ID NO: 14 - RNA, 22 nt, Artificial Sequence
  Synthetic Polynucleotide
cugggaaaaccuuuaccaacag

SEQ ID NO: 15 - RNA, 22 nt, Artificial Sequence
  Synthetic Polynucleotide
ucccaaccucagacagagagca

SEQ ID NO: 16 - RNA, 22 nt, Artificial Sequence
  Synthetic Polynucleotide
gauguuacugcugcgucgcucc

SEQ ID NO: 17 - RNA, 22 nt, Artificial Sequence
  Synthetic Polynucleotide
caugauccugacuguguucugu

SEQ ID NO: 18 - RNA, 22 nt, Artificial Sequence
  Synthetic Polynucleotide
cucguguguagucaguguccag

SEQ ID NO: 19 - RNA, 22 nt, Artificial Sequence
  Synthetic Polynucleotide
aaacugauugccauggauccau

SEQ ID NO: 20 - RNA, 22 nt, Artificial Sequence
  Synthetic Polynucleotide
agaaaacaaggagccacgaaug

SEQ ID NO: 21 - RNA, 20 nt, Artificial Sequence
  Synthetic Polynucleotide
gcuccccgaucaguucugcu

SEQ ID NO: 22 - RNA, 20 nt, Artificial Sequence
  Synthetic Polynucleotide
uguagucaccauggcguaug

SEQ ID NO: 23 - RNA, 20 nt, Artificial Sequence
  Synthetic Polynucleotide
ggaagcuccgcagcacagac

SEQ ID NO: 24 - RNA, 20 nt, Artificial Sequence
  Synthetic Polynucleotide
uccuuacaaccagcgcagga

SEQ ID NO: 25 - RNA, 20 nt, Artificial Sequence
  Synthetic Polynucleotide
acuucugaccccuuacugug

SEQ ID NO: 26 - RNA, 20 nt, Artificial Sequence
  Synthetic Polynucleotide
gagcucccagcagaacugau

SEQ ID NO: 27 - RNA, 20 nt, Artificial Sequence
  Synthetic Polynucleotide
ccgagacaucgacagcucca

SEQ ID NO: 28 - RNA, 20 nt, Artificial Sequence
  Synthetic Polynucleotide
auccguucuacagcacacac

SEQ ID NO: 29 - RNA, 20 nt, Artificial Sequence
  Synthetic Polynucleotide
ucacguaccugagagauccu

SEQ ID NO: 30 - RNA, 20 nt, Artificial Sequence
  Synthetic Polynucleotide
cgcaggugcuagcagcacua

SEQ ID NO: 31 - RNA, 22 nt, Artificial Sequence
  Synthetic Polynucleotide
cccuggagcugucgaugucucg

SEQ ID NO: 32 - RNA, 22 nt, Artificial Sequence
  Synthetic Polynucleotide
uagauccguucuacagcacaca

SEQ ID NO: 33 - RNA, 22 nt, Artificial Sequence
  Synthetic Polynucleotide
agugagaggaaagcccaagcaa

SEQ ID NO: 34 - RNA, 22 nt, Artificial Sequence
  Synthetic Polynucleotide
accuuuccgggcccaaagggca

SEQ ID NO: 35 - RNA, 22 nt, Artificial Sequence
  Synthetic Polynucleotide
cuuugacugcaucaucgucacu

SEQ ID NO: 36 - RNA, 22 nt, Artificial Sequence
  Synthetic Polynucleotide
cacuucuucuggaaauaauagu

SEQ ID NO: 37 - RNA, 22 nt, Artificial Sequence
  Synthetic Polynucleotide
auuuuagcgucauuacccuggc

SEQ ID NO: 38 - RNA, 22 nt, Artificial Sequence
  Synthetic Polynucleotide
aacaacuuccgucgcuuuacuc

SEQ ID NO: 39 - RNA, 22 nt, Artificial Sequence
  Synthetic Polynucleotide
gccgagauaucucacucccuga

SEQ ID NO: 40 - RNA, 22 nt, Artificial Sequence
  Synthetic Polynucleotide
ugguguucaucuucuccaugcc

SEQ ID NO: 41 - RNA, 80 nt, Artificial Sequence
  Synthetic Polynucleotide
guuuuaguacucuggaaacagaaucuacuaaaacaaggcaaaaugccguguuuaucucgucaacuuguuggcgagauuuu

SEQ ID NO: 42 - DNA, 86 bp, Artificial Sequence
  Synthetic Polynucleotide
gtttaagagctatgctggaaacagcatagcaagtttaaataaggctagtccgttatcaacttgaaaaagtggcaccgagtcggtgc

SEQ ID NO: 43 - RNA, 20 nt, Artificial Sequence
  Synthetic Polynucleotide
aucuauggggacauuccucc

SEQ ID NO: 44 - RNA, 20 nt, Artificial Sequence
  Synthetic Polynucleotide
cagcaaugcguuguucaaug

SEQ ID NO: 45 - RNA, 20 nt, Artificial Sequence
  Synthetic Polynucleotide
aguagggguccaaguccucc

SEQ ID NO: 46 - RNA, 20 nt, Artificial Sequence
  Synthetic Polynucleotide
uggggacauuccucccggca

SEQ ID NO: 47 - RNA, 20 nt, Artificial Sequence
  Synthetic Polynucleotide
guagggguccaaguccucca

SEQ ID NO: 48 - RNA, 20 nt, Artificial Sequence
  Synthetic Polynucleotide
uagggguccaaguccuccag

SEQ ID NO: 49 - RNA, 20 nt, Artificial Sequence
  Synthetic Polynucleotide
auggcaauguugccuccccc

SEQ ID NO: 50 - RNA, 20 nt, Artificial Sequence
  Synthetic Polynucleotide
ggcucugacaccaugccggg

SEQ ID NO: 51 - RNA, 20 nt, Artificial Sequence
  Synthetic Polynucleotide
aacagcugcccuucaucuau

SEQ ID NO: 52 - RNA, 20 nt, Artificial Sequence
  Synthetic Polynucleotide
cggcauggugucagagcccc

SEQ ID NO: 53 - RNA, 20 nt, Artificial Sequence
  Synthetic Polynucleotide
agcaaugcguuguucaauga

SEQ ID NO: 54 - RNA, 20 nt, Artificial Sequence
  Synthetic Polynucleotide
aggaauguccccauagauga

SEQ ID NO: 55 - RNA, 20 nt, Artificial Sequence
  Synthetic Polynucleotide
aaacagcugcccuucaucua

SEQ ID NO: 56 - RNA, 20 nt, Artificial Sequence
  Synthetic Polynucleotide
ccccuacuaugcagacaaaa

SEQ ID NO: 57 - RNA, 20 nt, Artificial Sequence
  Synthetic Polynucleotide
ccaugaauaacccaccggac

SEQ ID NO: 58 - RNA, 20 nt, Artificial Sequence
  Synthetic Polynucleotide
cauuuuugguccaguccggu

SEQ ID NO: 59 - RNA, 20 nt, Artificial Sequence
  Synthetic Polynucleotide
ccaguccgguggguuauuca

SEQ ID NO: 60 - RNA, 20 nt, Artificial Sequence
  Synthetic Polynucleotide
acauuuuugguccaguccgg

SEQ ID NO: 61 - RNA, 20 nt, Artificial Sequence
  Synthetic Polynucleotide
ucgacauuuuugguccaguc

SEQ ID NO: 62 - RNA, 20 nt, Artificial Sequence
  Synthetic Polynucleotide
acccacuuacucgacauuuu

SEQ ID NO: 63 - RNA, 20 nt, Artificial Sequence
  Synthetic Polynucleotide
uaugaccaugaauaacccac

SEQ ID NO: 64 - RNA, 20 nt, Artificial Sequence
  Synthetic Polynucleotide
uucuucgugacccguggaac

SEQ ID NO: 65 - RNA, 20 nt, Artificial Sequence
  Synthetic Polynucleotide
ucgugacccguggaacuggc

SEQ ID NO: 66 - RNA, 20 nt, Artificial Sequence
  Synthetic Polynucleotide
ucacuuuucuucgugacccg

SEQ ID NO: 67 - RNA, 20 nt, Artificial Sequence
  Synthetic Polynucleotide
guguuuagguacacuuuuac

SEQ ID NO: 68 - RNA, 20 nt, Artificial Sequence
  Synthetic Polynucleotide
aagccccuacaauugucuuc

SEQ ID NO: 69 - RNA, 20 nt, Artificial Sequence
  Synthetic Polynucleotide
cugaguguguuugcacuaau

SEQ ID NO: 70 - RNA, 20 nt, Artificial Sequence
  Synthetic Polynucleotide
auuggacuacagcuguucau

SEQ ID NO: 71 - RNA, 20 nt, Artificial Sequence
  Synthetic Polynucleotide
aggccugaagacaauuguag

SEQ ID NO: 72 - RNA, 20 nt, Artificial Sequence
  Synthetic Polynucleotide
aagcucguguagccauaauc

SEQ ID NO: 73 - RNA, 20 nt, Artificial Sequence
  Synthetic Polynucleotide
agcucguguagccauaauca

SEQ ID NO: 74 - RNA, 20 nt, Artificial Sequence
  Synthetic Polynucleotide
ggcuaaugacccaagauuac

SEQ ID NO: 75 - RNA, 20 nt, Artificial Sequence
  Synthetic Polynucleotide
uucacacagguguaccccuc

SEQ ID NO: 76 - RNA, 20 nt, Artificial Sequence
  Synthetic Polynucleotide
cguguguagucaguguccag

SEQ ID NO: 77 - RNA, 20 nt, Artificial Sequence
  Synthetic Polynucleotide
gcuaaugacccaagauuacu

SEQ ID NO: 78 - RNA, 20 nt, Artificial Sequence
  Synthetic Polynucleotide
cgagcuuugacacuuucagc

SEQ ID NO: 79 - RNA, 20 nt, Artificial Sequence
  Synthetic Polynucleotide
ugguacucaccuguugguaa

SEQ ID NO: 80 - RNA, 20 nt, Artificial Sequence
  Synthetic Polynucleotide
ggguacaccugugugaaaau

SEQ ID NO: 81 - RNA, 20 nt, Artificial Sequence
  Synthetic Polynucleotide
cugggaaaaccuuuaccaac

SEQ ID NO: 82 - RNA, 20 nt, Artificial Sequence
  Synthetic Polynucleotide
uugggucauuagccuaaaca

SEQ ID NO: 83 - RNA, 20 nt, Artificial Sequence
  Synthetic Polynucleotide
guguguagucaguguccaga

SEQ ID NO: 84 - RNA, 20 nt, Artificial Sequence
  Synthetic Polynucleotide
gggccuucuuagccuuguuu

SEQ ID NO: 85 - RNA, 20 nt, Artificial Sequence
  Synthetic Polynucleotide
gagcuuugacacuuucagcu

SEQ ID NO: 86 - RNA, 20 nt, Artificial Sequence
  Synthetic Polynucleotide
auuggcagaaacccugauua

SEQ ID NO: 87 - RNA, 20 nt, Artificial Sequence
  Synthetic Polynucleotide
cccuagacgcugcgugcugc

SEQ ID NO: 88 - RNA, 20 nt, Artificial Sequence
  Synthetic Polynucleotide
ccagcagcacgcagcgucua

SEQ ID NO: 89 - RNA, 20 nt, Artificial Sequence
  Synthetic Polynucleotide
gccagcagcacgcagcgucu

SEQ ID NO: 90 - RNA, 20 nt, Artificial Sequence
  Synthetic Polynucleotide
cuuugucguagugauuuucc

SEQ ID NO: 91 - RNA, 20 nt, Artificial Sequence
  Synthetic Polynucleotide
gagguugucuacccccaauc

SEQ ID NO: 92 - RNA, 20 nt, Artificial Sequence
  Synthetic Polynucleotide
uaugcccuucgacaccaagg

SEQ ID NO: 93 - RNA, 20 nt, Artificial Sequence
  Synthetic Polynucleotide
accuuggugucgaagggcau

SEQ ID NO: 94 - RNA, 20 nt, Artificial Sequence
  Synthetic Polynucleotide
aguuuccaccuuggugucga

SEQ ID NO: 95 - RNA, 20 nt, Artificial Sequence
  Synthetic Polynucleotide
guuuccaccuuggugucgaa

SEQ ID NO: 96 - RNA, 20 nt, Artificial Sequence
  Synthetic Polynucleotide
ccgcugccgcugcaauugcc

SEQ ID NO: 97 - RNA, 20 nt, Artificial Sequence
  Synthetic Polynucleotide
gcccaaccaggcaauugcag

SEQ ID NO: 98 - RNA, 20 nt, Artificial Sequence
  Synthetic Polynucleotide
aauuugggugguaccugauu

SEQ ID NO: 99 - RNA, 20 nt, Artificial Sequence
  Synthetic Polynucleotide
cguucaccggcagcauuggu

SEQ ID NO: 100 - RNA, 20 nt, Artificial Sequence
  Synthetic Polynucleotide
ccguucaccggcagcauugg

SEQ ID NO: 101 - RNA, 20 nt, Artificial Sequence
  Synthetic Polynucleotide
uguuacugcugcgucgcucc

SEQ ID NO: 102 - RNA, 20 nt, Artificial Sequence
  Synthetic Polynucleotide
guuacugcugcgucgcuccu

SEQ ID NO: 103 - RNA, 20 nt, Artificial Sequence
  Synthetic Polynucleotide
gggcugagcguccaucaacc

SEQ ID NO: 104 - RNA, 20 nt, Artificial Sequence
  Synthetic Polynucleotide
caccaaugcugccggugaac

SEQ ID NO: 105 - RNA, 20 nt, Artificial Sequence
  Synthetic Polynucleotide
ggcugagcguccaucaacca

SEQ ID NO: 106 - RNA, 20 nt, Artificial Sequence
  Synthetic Polynucleotide
uuacugcugcgucgcuccug

SEQ ID NO: 107 - RNA, 20 nt, Artificial Sequence
  Synthetic Polynucleotide
cugcaacgguguggucuccc

SEQ ID NO: 108 - RNA, 20 nt, Artificial Sequence
  Synthetic Polynucleotide
cagcauugguggggaccuac

SEQ ID NO: 109 - RNA, 20 nt, Artificial Sequence
  Synthetic Polynucleotide
gcgucgcuccuggggucugu

SEQ ID NO: 110 - RNA, 20 nt, Artificial Sequence
  Synthetic Polynucleotide
ugcuguggacugcaacggug

SEQ ID NO: 111 - RNA, 20 nt, Artificial Sequence
  Synthetic Polynucleotide
cgucgcuccuggggucugug

SEQ ID NO: 112 - RNA, 20 nt, Artificial Sequence
  Synthetic Polynucleotide
ugcgucgcuccuggggucug

SEQ ID NO: 113 - RNA, 20 nt, Artificial Sequence
  Synthetic Polynucleotide
gguguggucucccugguuga

SEQ ID NO: 114 - RNA, 20 nt, Artificial Sequence
  Synthetic Polynucleotide
guaacaucagccaagccagu

SEQ ID NO: 115 - RNA, 20 nt, Artificial Sequence
  Synthetic Polynucleotide
cugugcauuuucccguucac

SEQ ID NO: 116 - RNA, 20 nt, Artificial Sequence
  Synthetic Polynucleotide
guuugugccccacagacccc

SEQ ID NO: 117 - RNA, 20 nt, Artificial Sequence
  Synthetic Polynucleotide
gcaaaucuguaccaccaagg

SEQ ID NO: 118 - RNA, 20 nt, Artificial Sequence
  Synthetic Polynucleotide
ugugcaaaucuguaccacca

SEQ ID NO: 119 - RNA, 20 nt, Artificial Sequence
  Synthetic Polynucleotide
ccaagguggacauuuuuguc

SEQ ID NO: 120 - RNA, 20 nt, Artificial Sequence
  Synthetic Polynucleotide
ugucuggacucuucaaguuc

SEQ ID NO: 121 - RNA, 20 nt, Artificial Sequence
  Synthetic Polynucleotide
ucuggaauugcucuccauau

SEQ ID NO: 122 - RNA, 20 nt, Artificial Sequence
  Synthetic Polynucleotide
aguucugcgaucauucagac

SEQ ID NO: 123 - RNA, 20 nt, Artificial Sequence
  Synthetic Polynucleotide
ggagcucuuucuagcagaug

SEQ ID NO: 124 - RNA, 20 nt, Artificial Sequence
  Synthetic Polynucleotide
ccuacuuggaaauacucaua

SEQ ID NO: 125 - RNA, 22 nt, Artificial Sequence
  Synthetic Polynucleotide
ggggcucugacaccaugccggg

SEQ ID NO: 126 - RNA, 22 nt, Artificial Sequence
  Synthetic Polynucleotide
ccccuacuaugcagacaaaaag

SEQ ID NO: 127 - RNA, 22 nt, Artificial Sequence
  Synthetic Polynucleotide
cucaccuuuuugucugcauagu

SEQ ID NO: 128 - RNA, 22 nt, Artificial Sequence
  Synthetic Polynucleotide
aucaucaucuuucuuuucuucu

SEQ ID NO: 129 - RNA, 22 nt, Artificial Sequence
  Synthetic Polynucleotide
uuucuccuuucaguccucuaag

SEQ ID NO: 130 - RNA, 22 nt, Artificial Sequence
  Synthetic Polynucleotide
ucgacauuuuugguccaguccg

SEQ ID NO: 131 - RNA, 22 nt, Artificial Sequence
  Synthetic Polynucleotide
ccaccggacuggaccaaaaaug

SEQ ID NO: 132 - RNA, 22 nt, Artificial Sequence
  Synthetic Polynucleotide
gacaaacugcauauuuaugacc

SEQ ID NO: 133 - RNA, 22 nt, Artificial Sequence
  Synthetic Polynucleotide
aauagugcacaugaugagcaug

SEQ ID NO: 134 - RNA, 22 nt, Artificial Sequence
  Synthetic Polynucleotide
uggucauaaauaugcaguuugu

SEQ ID NO: 135 - RNA, 22 nt, Artificial Sequence
  Synthetic Polynucleotide
cuccuacacagaagccucuugc

SEQ ID NO: 136 - RNA, 22 nt, Artificial Sequence
  Synthetic Polynucleotide
ucuucgugacccguggaacugg

SEQ ID NO: 137 - RNA, 22 nt, Artificial Sequence
  Synthetic Polynucleotide
guuccacgggucacgaagaaaa

SEQ ID NO: 138 - RNA, 22 nt, Artificial Sequence
  Synthetic Polynucleotide
cuugcaagaggcuucuguguag

SEQ ID NO: 139 - RNA, 22 nt, Artificial Sequence
  Synthetic Polynucleotide
uuguguuuagguacacuuuuac

SEQ ID NO: 140 - RNA, 22 nt, Artificial Sequence
  Synthetic Polynucleotide
acuuuuacuggaauauauacuu

SEQ ID NO: 141 - RNA, 22 nt, Artificial Sequence
  Synthetic Polynucleotide
uacaaauucuguuaaauaccug

SEQ ID NO: 142 - RNA, 22 nt, Artificial Sequence
  Synthetic Polynucleotide
auuuaauucuacagguauuuaa

SEQ ID NO: 143 - RNA, 22 nt, Artificial Sequence
  Synthetic Polynucleotide
uucuaaagucuucuucacucuc

SEQ ID NO: 144 - RNA, 22 nt, Artificial Sequence
  Synthetic Polynucleotide
gagugaagaagacuuuagaagu

SEQ ID NO: 145 - RNA, 22 nt, Artificial Sequence
  Synthetic Polynucleotide
agaaagcauaaugaauacccua

SEQ ID NO: 146 - RNA, 22 nt, Artificial Sequence
  Synthetic Polynucleotide
acacacucagacagaacacagu

SEQ ID NO: 147 - RNA, 22 nt, Artificial Sequence
  Synthetic Polynucleotide
uaaugaaacauuagaaagcaua

SEQ ID NO: 148 - RNA, 22 nt, Artificial Sequence
  Synthetic Polynucleotide
ucuaauguuucauuauuuucaa

SEQ ID NO: 149 - RNA, 22 nt, Artificial Sequence
  Synthetic Polynucleotide
gaaaccacaaaggagagcaucu

SEQ ID NO: 150 - RNA, 22 nt, Artificial Sequence
  Synthetic Polynucleotide
cuuugugguuucagcacagauu

SEQ ID NO: 151 - RNA, 22 nt, Artificial Sequence
  Synthetic Polynucleotide
acagaauauuuuuauuacuugg

SEQ ID NO: 152 - RNA, 22 nt, Artificial Sequence
  Synthetic Polynucleotide
ucaaagcucguguagccauaau

SEQ ID NO: 153 - RNA, 22 nt, Artificial Sequence
  Synthetic Polynucleotide
gguaaagguuuucccaguaauc

SEQ ID NO: 154 - RNA, 22 nt, Artificial Sequence
  Synthetic Polynucleotide
aacauugaagaagcuaaacaga

SEQ ID NO: 155 - RNA, 22 nt, Artificial Sequence
  Synthetic Polynucleotide
cauaugccauggcaaccacagc

SEQ ID NO: 156 - RNA, 22 nt, Artificial Sequence
  Synthetic Polynucleotide
gaagaagcuaaacagaaagaau

SEQ ID NO: 157 - RNA, 22 nt, Artificial Sequence
  Synthetic Polynucleotide
gcaauuugggugguaccugauu

SEQ ID NO: 158 - RNA, 22 nt, Artificial Sequence
  Synthetic Polynucleotide
caggcaauugcagcggcagcgg

SEQ ID NO: 159 - RNA, 22 nt, Artificial Sequence
  Synthetic Polynucleotide
uuuagcacuuuuagagcucagu

SEQ ID NO: 160 - RNA, 22 nt, Artificial Sequence
  Synthetic Polynucleotide
gaugcugagaaauugucgaaau

SEQ ID NO: 161 - RNA, 22 nt, Artificial Sequence
  Synthetic Polynucleotide
aauauacaaguauuaggagaag

SEQ ID NO: 162 - RNA, 22 nt, Artificial Sequence
  Synthetic Polynucleotide
ggaagagauauaggaucugaga

SEQ ID NO: 163 - RNA, 22 nt, Artificial Sequence
  Synthetic Polynucleotide
uucaaaggcagaggaagagaua

SEQ ID NO: 164 - RNA, 22 nt, Artificial Sequence
  Synthetic Polynucleotide
cuaucuccuuucagaggauaug

SEQ ID NO: 165 - RNA, 22 nt, Artificial Sequence
  Synthetic Polynucleotide
cucauugcucucugucugaggu

SEQ ID NO: 166 - RNA, 22 nt, Artificial Sequence
  Synthetic Polynucleotide
uuguaguuccuaucuccuuuca

SEQ ID NO: 167 - RNA, 22 nt, Artificial Sequence
  Synthetic Polynucleotide
auggaacaccacccaaugacug

SEQ ID NO: 168 - RNA, 22 nt, Artificial Sequence
  Synthetic Polynucleotide
auauggagagcaauuccagauc

SEQ ID NO: 169 - RNA, 22 nt, Artificial Sequence
  Synthetic Polynucleotide
ugaucuggaauugcucuccaua

SEQ ID NO: 170 - RNA, 22 nt, Artificial Sequence
  Synthetic Polynucleotide
augguaauugcaagaucuacaa

SEQ ID NO: 171 - RNA, 22 nt, Artificial Sequence
  Synthetic Polynucleotide
ugcuuuuuucucccagaacuug

SEQ ID NO: 172 - RNA, 22 nt, Artificial Sequence
  Synthetic Polynucleotide
auuuccuauagcaaguacauuu

SEQ ID NO: 173 - RNA, 22 nt, Artificial Sequence
  Synthetic Polynucleotide
acauuuuugaauuccucaguca

SEQ ID NO: 174 - RNA, 22 nt, Artificial Sequence
  Synthetic Polynucleotide
auuugcacacaaauucuugauc

SEQ ID NO: 175 - RNA, 22 nt, Artificial Sequence
  Synthetic Polynucleotide
aaaguguaucuauuuuauugua

SEQ ID NO: 176 - RNA, 22 nt, Artificial Sequence
  Synthetic Polynucleotide
uacaauaaaauagauacacuuu

SEQ ID NO: 177 - RNA, 22 nt, Artificial Sequence
  Synthetic Polynucleotide
aaugguauuaaaacugauugcc

SEQ ID NO: 178 - RNA, 22 nt, Artificial Sequence
  Synthetic Polynucleotide
auaugaguauuuccaaguaggc

SEQ ID NO: 179 - RNA, 22 nt, Artificial Sequence
  Synthetic Polynucleotide
accuuaguuuauguuuaccagu

SEQ ID NO: 180 - RNA, 22 nt, Artificial Sequence
  Synthetic Polynucleotide
cagccuacuuggaaauacucau

SEQ ID NO: 181 - RNA, 22 nt, Artificial Sequence
  Synthetic Polynucleotide
gagcucuuucuagcagaugugg

SEQ ID NO: 182 - RNA, 22 nt, Artificial Sequence
  Synthetic Polynucleotide
uuuuucucacuuaggucuuuac

SEQ ID NO: 183 - RNA, 20 nt, Artificial Sequence
  Synthetic Polynucleotide
gacggaaguuguuaguuucg

SEQ ID NO: 184 - RNA, 20 nt, Artificial Sequence
  Synthetic Polynucleotide
caacuuccgucgcuuuacuc

SEQ ID NO: 185 - RNA, 20 nt, Artificial Sequence
  Synthetic Polynucleotide
ucgcuuuacuccggagucac

SEQ ID NO: 186 - RNA, 20 nt, Artificial Sequence
  Synthetic Polynucleotide
gaacggaucuagauccucca

SEQ ID NO: 187 - RNA, 20 nt, Artificial Sequence
  Synthetic Polynucleotide
acggaaguuguuaguuucga

SEQ ID NO: 188 - RNA, 20 nt, Artificial Sequence
  Synthetic Polynucleotide
aacggaucuagauccuccag

SEQ ID NO: 189 - RNA, 20 nt, Artificial Sequence
  Synthetic Polynucleotide
agaacggaucuagauccucc

SEQ ID NO: 190 - RNA, 20 nt, Artificial Sequence
  Synthetic Polynucleotide
gugacuccggaguaaagcga

SEQ ID NO: 191 - RNA, 20 nt, Artificial Sequence
  Synthetic Polynucleotide
uuaguuucgagggauccaau

SEQ ID NO: 192 - RNA, 20 nt, Artificial Sequence
  Synthetic Polynucleotide
guuaguuucgagggauccaa

SEQ ID NO: 193 - RNA, 20 nt, Artificial Sequence
  Synthetic Polynucleotide
ggcuccccgaucaguucugc

SEQ ID NO: 194 - RNA, 20 nt, Artificial Sequence
  Synthetic Polynucleotide
uaguuucgagggauccaaug

SEQ ID NO: 195 - RNA, 20 nt, Artificial Sequence
  Synthetic Polynucleotide
gcucccagcagaacugaucg

SEQ ID NO: 196 - RNA, 20 nt, Artificial Sequence
  Synthetic Polynucleotide
acccggugugugcuguagaa

SEQ ID NO: 197 - RNA, 20 nt, Artificial Sequence
  Synthetic Polynucleotide
agaacugaucggggagcccc

SEQ ID NO: 198 - RNA, 20 nt, Artificial Sequence
  Synthetic Polynucleotide
uccguucuacagcacacacc

SEQ ID NO: 199 - RNA, 20 nt, Artificial Sequence
  Synthetic Polynucleotide
cuuuacuccggagucacugg

SEQ ID NO: 200 - RNA, 20 nt, Artificial Sequence
  Synthetic Polynucleotide
caccauagaacuugggcagc

SEQ ID NO: 201 - RNA, 20 nt, Artificial Sequence
  Synthetic Polynucleotide
ugggagcucaccauagaacu

SEQ ID NO: 202 - RNA, 20 nt, Artificial Sequence
  Synthetic Polynucleotide
acugaucggggagccccugg

SEQ ID NO: 203 - RNA, 20 nt, Artificial Sequence
  Synthetic Polynucleotide
aaccagcugcccaaguucua

SEQ ID NO: 204 - RNA, 20 nt, Artificial Sequence
  Synthetic Polynucleotide
gaacuugggcagcugguugc

SEQ ID NO: 205 - RNA, 20 nt, Artificial Sequence
  Synthetic Polynucleotide
gaagcaaauugcugccaagc

SEQ ID NO: 206 - RNA, 20 nt, Artificial Sequence
  Synthetic Polynucleotide
ucuaucuccaccagugacuc

SEQ ID NO: 207 - RNA, 20 nt, Artificial Sequence
  Synthetic Polynucleotide
ggggccgaggcuucucuucu

SEQ ID NO: 208 - RNA, 20 nt, Artificial Sequence
  Synthetic Polynucleotide
gggcccgaguggcacuaaac

SEQ ID NO: 209 - RNA, 20 nt, Artificial Sequence
  Synthetic Polynucleotide
uucccgguuuagugccacuc

SEQ ID NO: 210 - RNA, 20 nt, Artificial Sequence
  Synthetic Polynucleotide
ggcccgaguggcacuaaacc

SEQ ID NO: 211 - RNA, 20 nt, Artificial Sequence
  Synthetic Polynucleotide
uuucccgguuuagugccacu

SEQ ID NO: 212 - RNA, 20 nt, Artificial Sequence
  Synthetic Polynucleotide
uuagugccacucgggcccug

SEQ ID NO: 213 - RNA, 20 nt, Artificial Sequence
  Synthetic Polynucleotide
ugauggccguucuucugauc

SEQ ID NO: 214 - RNA, 20 nt, Artificial Sequence
  Synthetic Polynucleotide
gaauagccacagggcccgag

SEQ ID NO: 215 - RNA, 20 nt, Artificial Sequence
  Synthetic Polynucleotide
guucuucugaucagguugaa

SEQ ID NO: 216 - RNA, 20 nt, Artificial Sequence
  Synthetic Polynucleotide
aguggcacuaaaccgggaaa

SEQ ID NO: 217 - RNA, 20 nt, Artificial Sequence
  Synthetic Polynucleotide
acaaagggaggaccauuucc

SEQ ID NO: 218 - RNA, 20 nt, Artificial Sequence
  Synthetic Polynucleotide
ggauuuuagcgucauuaccc

SEQ ID NO: 219 - RNA, 20 nt, Artificial Sequence
  Synthetic Polynucleotide
cauagagauauacucacgcc

SEQ ID NO: 220 - RNA, 20 nt, Artificial Sequence
  Synthetic Polynucleotide
gccaguuccaaggaucucuc

SEQ ID NO: 221 - RNA, 20 nt, Artificial Sequence
  Synthetic Polynucleotide
accugagagauccuuggaac

SEQ ID NO: 222 - RNA, 20 nt, Artificial Sequence
  Synthetic Polynucleotide
gcacagcaauagaucuccgu

SEQ ID NO: 223 - RNA, 20 nt, Artificial Sequence
  Synthetic Polynucleotide
uaagaacucugaauguccgc

SEQ ID NO: 224 - RNA, 20 nt, Artificial Sequence
  Synthetic Polynucleotide
auagaucuccgugggaucuc

SEQ ID NO: 225 - RNA, 20 nt, Artificial Sequence
  Synthetic Polynucleotide
ggcacagcaauagaucuccg

SEQ ID NO: 226 - RNA, 20 nt, Artificial Sequence
  Synthetic Polynucleotide
gggcccccacaaugaccuuc

SEQ ID NO: 227 - RNA, 20 nt, Artificial Sequence
  Synthetic Polynucleotide
cgcaggccugaaggucauug

SEQ ID NO: 228 - RNA, 20 nt, Artificial Sequence
  Synthetic Polynucleotide
guggggcugcaacucuucaa

SEQ ID NO: 229 - RNA, 20 nt, Artificial Sequence
  Synthetic Polynucleotide
gcaggccugaaggucauugu

SEQ ID NO: 230 - RNA, 20 nt, Artificial Sequence
  Synthetic Polynucleotide
gguggggcugcaacucuuca

SEQ ID NO: 231 - RNA, 20 nt, Artificial Sequence
  Synthetic Polynucleotide
ucaucuucccgcaggccuga

SEQ ID NO: 232 - RNA, 20 nt, Artificial Sequence
  Synthetic Polynucleotide
gaagaguugcagccccacca

SEQ ID NO: 233 - RNA, 20 nt, Artificial Sequence
  Synthetic Polynucleotide
gaccccuuacuguguggcaa

SEQ ID NO: 234 - RNA, 20 nt, Artificial Sequence
  Synthetic Polynucleotide
agauccauugccacacagua

SEQ ID NO: 235 - RNA, 20 nt, Artificial Sequence
  Synthetic Polynucleotide
uguggcaauggaucugacuc

SEQ ID NO: 236 - RNA, 20 nt, Artificial Sequence
  Synthetic Polynucleotide
guggcaauggaucugacuca

SEQ ID NO: 237 - RNA, 20 nt, Artificial Sequence
  Synthetic Polynucleotide
gauccauugccacacaguaa

SEQ ID NO: 238 - RNA, 20 nt, Artificial Sequence
  Synthetic Polynucleotide
auccauugccacacaguaag

SEQ ID NO: 239 - RNA, 20 nt, Artificial Sequence
  Synthetic Polynucleotide
acuguuccgccucaugacac

SEQ ID NO: 240 - RNA, 20 nt, Artificial Sequence
  Synthetic Polynucleotide
cugggaacgccucuaccagc

SEQ ID NO: 241 - RNA, 20 nt, Artificial Sequence
  Synthetic Polynucleotide
ccggguugucagaaguuuua

SEQ ID NO: 242 - RNA, 20 nt, Artificial Sequence
  Synthetic Polynucleotide
gccucaugacacaggauucc

SEQ ID NO: 243 - RNA, 20 nt, Artificial Sequence
  Synthetic Polynucleotide
agcucaguaccugcugguag

SEQ ID NO: 244 - RNA, 20 nt, Artificial Sequence
  Synthetic Polynucleotide
uuaaggcagauauaaccauc

SEQ ID NO: 245 - RNA, 20 nt, Artificial Sequence
  Synthetic Polynucleotide
aagcugguguaguuaaaauc

SEQ ID NO: 246 - RNA, 20 nt, Artificial Sequence
  Synthetic Polynucleotide
ucaugaggcggaacagugag

SEQ ID NO: 247 - RNA, 20 nt, Artificial Sequence
  Synthetic Polynucleotide
ccuuaaaacuucugacaacc

SEQ ID NO: 248 - RNA, 20 nt, Artificial Sequence
  Synthetic Polynucleotide
gaaugcagcucaguaccugc

SEQ ID NO: 249 - RNA, 20 nt, Artificial Sequence
  Synthetic Polynucleotide
ggcccucgagaugcuccgga

SEQ ID NO: 250 - RNA, 20 nt, Artificial Sequence
  Synthetic Polynucleotide
guucugcuccucauacgcca

SEQ ID NO: 251 - RNA, 20 nt, Artificial Sequence
  Synthetic Polynucleotide
cuccuuccggagcaucucga

SEQ ID NO: 252 - RNA, 20 nt, Artificial Sequence
  Synthetic Polynucleotide
cuaccuggucaacuugaucu

SEQ ID NO: 253 - RNA, 20 nt, Artificial Sequence
  Synthetic Polynucleotide
gcuccuuccggagcaucucg

SEQ ID NO: 254 - RNA, 20 nt, Artificial Sequence
  Synthetic Polynucleotide
cgagaugcuccggaaggagc

SEQ ID NO: 255 - RNA, 20 nt, Artificial Sequence
  Synthetic Polynucleotide
aggaggcccucgagaugcuc

SEQ ID NO: 256 - RNA, 20 nt, Artificial Sequence
  Synthetic Polynucleotide
uuugugcucguaaucuuccu

SEQ ID NO: 257 - RNA, 20 nt, Artificial Sequence
  Synthetic Polynucleotide
ggagcaucucgagggccucc

SEQ ID NO: 258 - RNA, 20 nt, Artificial Sequence
  Synthetic Polynucleotide
ggcguaugaggagcagaacc

SEQ ID NO: 259 - RNA, 20 nt, Artificial Sequence
  Synthetic Polynucleotide
uuuugugcucguaaucuucc

SEQ ID NO: 260 - RNA, 20 nt, Artificial Sequence
  Synthetic Polynucleotide
ugaccagguagaaagauccc

SEQ ID NO: 261 - RNA, 20 nt, Artificial Sequence
  Synthetic Polynucleotide
gaucuuggcuguagucacca

SEQ ID NO: 262 - RNA, 20 nt, Artificial Sequence
  Synthetic Polynucleotide
accauccugcgcugguugua

SEQ ID NO: 263 - RNA, 20 nt, Artificial Sequence
  Synthetic Polynucleotide
cugauccuuacaaccagcgc

SEQ ID NO: 264 - RNA, 20 nt, Artificial Sequence
  Synthetic Polynucleotide
ucgcaggugcuagcagcacu

SEQ ID NO: 265 - RNA, 20 nt, Artificial Sequence
  Synthetic Polynucleotide
gguuaaaggugauccauugu

SEQ ID NO: 266 - RNA, 20 nt, Artificial Sequence
  Synthetic Polynucleotide
agccucuuaccauccugcgc

SEQ ID NO: 267 - RNA, 20 nt, Artificial Sequence
  Synthetic Polynucleotide
agguuaaaggugauccauug

SEQ ID NO: 268 - RNA, 20 nt, Artificial Sequence
  Synthetic Polynucleotide
ugguuguaaggaucagagcg

SEQ ID NO: 269 - RNA, 20 nt, Artificial Sequence
  Synthetic Polynucleotide
guggagcccucugacacucu

SEQ ID NO: 270 - RNA, 20 nt, Artificial Sequence
  Synthetic Polynucleotide
gcucacuagugggcggcggu

SEQ ID NO: 271 - RNA, 20 nt, Artificial Sequence
  Synthetic Polynucleotide
ggaaaacgccgggcuaguca

SEQ ID NO: 272 - RNA, 20 nt, Artificial Sequence
  Synthetic Polynucleotide
acuagcccggcguuuuccag

SEQ ID NO: 273 - RNA, 20 nt, Artificial Sequence
  Synthetic Polynucleotide
accgagacaucgacagcucc

SEQ ID NO: 274 - RNA, 20 nt, Artificial Sequence
  Synthetic Polynucleotide
cccggcguuuuccagaggcg

SEQ ID NO: 275 - RNA, 20 nt, Artificial Sequence
  Synthetic Polynucleotide
gagagccccgauggcuuucg

SEQ ID NO: 276 - RNA, 20 nt, Artificial Sequence
  Synthetic Polynucleotide
cgauggcuuucguggucucc

SEQ ID NO: 277 - RNA, 20 nt, Artificial Sequence
  Synthetic Polynucleotide
ccucgccucuggaaaacgcc

SEQ ID NO: 278 - RNA, 20 nt, Artificial Sequence
  Synthetic Polynucleotide
cgagacaucgacagcuccag

SEQ ID NO: 279 - RNA, 20 nt, Artificial Sequence
  Synthetic Polynucleotide
gccucgccucuggaaaacgc

SEQ ID NO: 280 - RNA, 20 nt, Artificial Sequence
  Synthetic Polynucleotide
gggagugagauaucucggcc

SEQ ID NO: 281 - RNA, 20 nt, Artificial Sequence
  Synthetic Polynucleotide
ggagaccacgaaagccaucg

SEQ ID NO: 282 - RNA, 20 nt, Artificial Sequence
  Synthetic Polynucleotide
cgagauaucucacucccuga

SEQ ID NO: 283 - RNA, 20 nt, Artificial Sequence
  Synthetic Polynucleotide
cccacuagugagcuugcccc

SEQ ID NO: 284 - RNA, 20 nt, Artificial Sequence
  Synthetic Polynucleotide
ccaggggcaagcucacuagu

SEQ ID NO: 285 - RNA, 20 nt, Artificial Sequence
  Synthetic Polynucleotide
gagugagauaucucggccag

SEQ ID NO: 286 - RNA, 20 nt, Artificial Sequence
  Synthetic Polynucleotide
ccgagauaucucacucccug

SEQ ID NO: 287 - RNA, 20 nt, Artificial Sequence
  Synthetic Polynucleotide
cucggccaggggaccggaaa

SEQ ID NO: 288 - RNA, 20 nt, Artificial Sequence
  Synthetic Polynucleotide
agauaucucggccaggggac

SEQ ID NO: 289 - RNA, 20 nt, Artificial Sequence
  Synthetic Polynucleotide
guguuccauuuccggucccc

SEQ ID NO: 290 - RNA, 20 nt, Artificial Sequence
  Synthetic Polynucleotide
uccaggggcaagcucacuag

SEQ ID NO: 291 - RNA, 20 nt, Artificial Sequence
  Synthetic Polynucleotide
ggggcaagcucacuaguggg

SEQ ID NO: 292 - RNA, 20 nt, Artificial Sequence
  Synthetic Polynucleotide
ggagugagauaucucggcca

SEQ ID NO: 293 - RNA, 20 nt, Artificial Sequence
  Synthetic Polynucleotide
uggagaccacgaaagccauc

SEQ ID NO: 294 - RNA, 20 nt, Artificial Sequence
  Synthetic Polynucleotide
ggcgaggccuagaaaagacu

SEQ ID NO: 295 - RNA, 20 nt, Artificial Sequence
  Synthetic Polynucleotide
cccuggagcugucgaugucu

SEQ ID NO: 296 - RNA, 20 nt, Artificial Sequence
  Synthetic Polynucleotide
cuggagaccacgaaagccau

SEQ ID NO: 297 - RNA, 20 nt, Artificial Sequence
  Synthetic Polynucleotide
ucuuuucuaggccucgccuc

SEQ ID NO: 298 - RNA, 20 nt, Artificial Sequence
  Synthetic Polynucleotide
aggcgaggccuagaaaagac

SEQ ID NO: 299 - RNA, 20 nt, Artificial Sequence
  Synthetic Polynucleotide
ccucagggagugagauaucu

SEQ ID NO: 300 - RNA, 20 nt, Artificial Sequence
  Synthetic Polynucleotide
guugaggaagagggcuucua

SEQ ID NO: 301 - RNA, 20 nt, Artificial Sequence
  Synthetic Polynucleotide
uucuagggagggggccuugc

SEQ ID NO: 302 - RNA, 20 nt, Artificial Sequence
  Synthetic Polynucleotide
ucucaacaggcauucgaugc

SEQ ID NO: 303 - RNA, 20 nt, Artificial Sequence
  Synthetic Polynucleotide
augaaccuuuccgggcccaa

SEQ ID NO: 304 - RNA, 20 nt, Artificial Sequence
  Synthetic Polynucleotide
gcacuuacccucaaggacgg

SEQ ID NO: 305 - RNA, 20 nt, Artificial Sequence
  Synthetic Polynucleotide
uaucauaaccuccguccuug

SEQ ID NO: 306 - RNA, 20 nt, Artificial Sequence
  Synthetic Polynucleotide
ugaaccuuuccgggcccaaa

SEQ ID NO: 307 - RNA, 20 nt, Artificial Sequence
  Synthetic Polynucleotide
aucauaaccuccguccuuga

SEQ ID NO: 308 - RNA, 20 nt, Artificial Sequence
  Synthetic Polynucleotide
auugcccuuugggcccggaa

SEQ ID NO: 309 - RNA, 20 nt, Artificial Sequence
  Synthetic Polynucleotide
gugagcagcacuuacccuca

SEQ ID NO: 310 - RNA, 20 nt, Artificial Sequence
  Synthetic Polynucleotide
gcagcacuuacccucaagga

SEQ ID NO: 311 - RNA, 20 nt, Artificial Sequence
  Synthetic Polynucleotide
cacucauugcccuuugggcc

SEQ ID NO: 312 - RNA, 20 nt, Artificial Sequence
  Synthetic Polynucleotide
gacaacacucauugcccuuu

SEQ ID NO: 313 - RNA, 20 nt, Artificial Sequence
  Synthetic Polynucleotide
auacuuagaugaaccuuucc

SEQ ID NO: 314 - RNA, 20 nt, Artificial Sequence
  Synthetic Polynucleotide
ugacaacacucauugcccuu

SEQ ID NO: 315 - RNA, 20 nt, Artificial Sequence
  Synthetic Polynucleotide
cacucacgauguugccuauc

SEQ ID NO: 316 - RNA, 20 nt, Artificial Sequence
  Synthetic Polynucleotide
uucgaagccaugcuccagau

SEQ ID NO: 317 - RNA, 20 nt, Artificial Sequence
  Synthetic Polynucleotide
caccaucaccuugugcaucg

SEQ ID NO: 318 - RNA, 20 nt, Artificial Sequence
  Synthetic Polynucleotide
acacuauaaugcagaacucg

SEQ ID NO: 319 - RNA, 20 nt, Artificial Sequence
  Synthetic Polynucleotide
caccacgaugcacaagguga

SEQ ID NO: 320 - RNA, 20 nt, Artificial Sequence
  Synthetic Polynucleotide
cguggugaacaccaucuuca

SEQ ID NO: 321 - RNA, 20 nt, Artificial Sequence
  Synthetic Polynucleotide
ggagcauggcuucgaaggua

SEQ ID NO: 322 - RNA, 20 nt, Artificial Sequence
  Synthetic Polynucleotide
uaucuggagcauggcuucga

SEQ ID NO: 323 - RNA, 20 nt, Artificial Sequence
  Synthetic Polynucleotide
uggagcauggcuucgaaggu

SEQ ID NO: 324 - RNA, 20 nt, Artificial Sequence
  Synthetic Polynucleotide
cgaagguagggcucaugcca

SEQ ID NO: 325 - RNA, 20 nt, Artificial Sequence
  Synthetic Polynucleotide
gguguucaccacgaugcaca

SEQ ID NO: 326 - RNA, 20 nt, Artificial Sequence
  Synthetic Polynucleotide
acaagcuggucaagcagggu

SEQ ID NO: 327 - RNA, 20 nt, Artificial Sequence
  Synthetic Polynucleotide
gauguugccuaucuggagca

SEQ ID NO: 328 - RNA, 20 nt, Artificial Sequence
  Synthetic Polynucleotide
uucauggccauggagcacca

SEQ ID NO: 329 - RNA, 20 nt, Artificial Sequence
  Synthetic Polynucleotide
ucuugagcuucacccacaug

SEQ ID NO: 330 - RNA, 20 nt, Artificial Sequence
  Synthetic Polynucleotide
cugggauugcugccccaugu

SEQ ID NO: 331 - RNA, 20 nt, Artificial Sequence
  Synthetic Polynucleotide
ugucuugagcuucacccaca

SEQ ID NO: 332 - RNA, 20 nt, Artificial Sequence
  Synthetic Polynucleotide
gugauggugagcucugcaaa

SEQ ID NO: 333 - RNA, 20 nt, Artificial Sequence
  Synthetic Polynucleotide
ggugauggugagcucugcaa

SEQ ID NO: 334 - RNA, 20 nt, Artificial Sequence
  Synthetic Polynucleotide
ugugcugcggagcuuccgcu

SEQ ID NO: 335 - RNA, 20 nt, Artificial Sequence
  Synthetic Polynucleotide
gaaauaauaguaugggucga

SEQ ID NO: 336 - RNA, 20 nt, Artificial Sequence
  Synthetic Polynucleotide
acugugagucugcuagagcu

SEQ ID NO: 337 - RNA, 20 nt, Artificial Sequence
  Synthetic Polynucleotide
cacugugagucugcuagagc

SEQ ID NO: 338 - RNA, 22 nt, Artificial Sequence
  Synthetic Polynucleotide
gcgacggaaguuguuaguuucg

SEQ ID NO: 339 - RNA, 22 nt, Artificial Sequence
  Synthetic Polynucleotide
guuaguuucgagggauccaaug

SEQ ID NO: 340 - RNA, 22 nt, Artificial Sequence
  Synthetic Polynucleotide
cuuacccggugugugcuguaga

SEQ ID NO: 341 - RNA, 22 nt, Artificial Sequence
  Synthetic Polynucleotide
agaacugaucggggagccccug

SEQ ID NO: 342 - RNA, 22 nt, Artificial Sequence
  Synthetic Polynucleotide
ucucuaucuccaccagugacuc

SEQ ID NO: 343 - RNA, 22 nt, Artificial Sequence
  Synthetic Polynucleotide
aaugagaagauggaauucccca

SEQ ID NO: 344 - RNA, 22 nt, Artificial Sequence
  Synthetic Polynucleotide
aaggacugaauagccacagggc

SEQ ID NO: 345 - RNA, 22 nt, Artificial Sequence
  Synthetic Polynucleotide
ucuucugaucagguugaaagga

SEQ ID NO: 346 - RNA, 22 nt, Artificial Sequence
  Synthetic Polynucleotide
cugaccuuccagagaaaauuga

SEQ ID NO: 347 - RNA, 22 nt, Artificial Sequence
  Synthetic Polynucleotide
caauuuucucuggaaggucagu

SEQ ID NO: 348 - RNA, 22 nt, Artificial Sequence
  Synthetic Polynucleotide
cgaacugaccuuccagagaaaa

SEQ ID NO: 349 - RNA, 22 nt, Artificial Sequence
  Synthetic Polynucleotide
cuggcaagaggauuuugucuaa

SEQ ID NO: 350 - RNA, 22 nt, Artificial Sequence
  Synthetic Polynucleotide
acgcuaaaauccagccaguucc

SEQ ID NO: 351 - RNA, 22 nt, Artificial Sequence
  Synthetic Polynucleotide
ccugagagauccuuggaacugg

SEQ ID NO: 352 - RNA, 22 nt, Artificial Sequence
  Synthetic Polynucleotide
gccuugauaaagauacuggcaa

SEQ ID NO: 353 - RNA, 22 nt, Artificial Sequence
  Synthetic Polynucleotide
uuggcacagcaauagaucuccg

SEQ ID NO: 354 - RNA, 22 nt, Artificial Sequence
  Synthetic Polynucleotide
ggaucucaggccugcggacauu

SEQ ID NO: 355 - RNA, 22 nt, Artificial Sequence
  Synthetic Polynucleotide
uguuuuuaaugcucuaagaacu

SEQ ID NO: 356 - RNA, 22 nt, Artificial Sequence
  Synthetic Polynucleotide
cuccacucacguuuucugugag

SEQ ID NO: 357 - RNA, 22 nt, Artificial Sequence
  Synthetic Polynucleotide
aaacacuuaggcagaagauggu

SEQ ID NO: 358 - RNA, 22 nt, Artificial Sequence
  Synthetic Polynucleotide
caucagccaguuucuucacuga

SEQ ID NO: 359 - RNA, 22 nt, Artificial Sequence
  Synthetic Polynucleotide
aacuacucaucucacagaaaac

SEQ ID NO: 360 - RNA, 22 nt, Artificial Sequence
  Synthetic Polynucleotide
gucacaucagccaguuucuuca

SEQ ID NO: 361 - RNA, 22 nt, Artificial Sequence
  Synthetic Polynucleotide
caaccucaaaaauaaauguguc

SEQ ID NO: 362 - RNA, 22 nt, Artificial Sequence
  Synthetic Polynucleotide
uuuauuuuugagguugcccuug

SEQ ID NO: 363 - RNA, 22 nt, Artificial Sequence
  Synthetic Polynucleotide
ucagauccauugccacacagua

SEQ ID NO: 364 - RNA, 22 nt, Artificial Sequence
  Synthetic Polynucleotide
cuguguggcaauggaucugacu

SEQ ID NO: 365 - RNA, 22 nt, Artificial Sequence
  Synthetic Polynucleotide
guggcaauggaucugacucagg

SEQ ID NO: 366 - RNA, 22 nt, Artificial Sequence
  Synthetic Polynucleotide
ucugaccccuuacuguguggca

SEQ ID NO: 367 - RNA, 22 nt, Artificial Sequence
  Synthetic Polynucleotide
accugcugguagaggcguuccc

SEQ ID NO: 368 - RNA, 22 nt, Artificial Sequence
  Synthetic Polynucleotide
cucacuguuccgccucaugaca

SEQ ID NO: 369 - RNA, 22 nt, Artificial Sequence
  Synthetic Polynucleotide
cugccuuaaaacuucugacaac

SEQ ID NO: 370 - RNA, 22 nt, Artificial Sequence
  Synthetic Polynucleotide
ucaaagcugguguaguuaaaau

SEQ ID NO: 371 - RNA, 22 nt, Artificial Sequence
  Synthetic Polynucleotide
uuuuuugugcucguaaucuucc

SEQ ID NO: 372 - RNA, 22 nt, Artificial Sequence
  Synthetic Polynucleotide
uauagauuuucccagaaguccu

SEQ ID NO: 373 - RNA, 22 nt, Artificial Sequence
  Synthetic Polynucleotide
gcucugauccuuacaaccagcg

SEQ ID NO: 374 - RNA, 22 nt, Artificial Sequence
  Synthetic Polynucleotide
cuucgcaggugcuagcagcacu

SEQ ID NO: 375 - RNA, 22 nt, Artificial Sequence
  Synthetic Polynucleotide
cuuaccauccugcgcugguugu

SEQ ID NO: 376 - RNA, 22 nt, Artificial Sequence
  Synthetic Polynucleotide
gcgcugguuguaaggaucagag

SEQ ID NO: 377 - RNA, 22 nt, Artificial Sequence
  Synthetic Polynucleotide
acaaccucucuccacucccaca

SEQ ID NO: 378 - RNA, 22 nt, Artificial Sequence
  Synthetic Polynucleotide
gagguuaaaggugauccauugu

SEQ ID NO: 379 - RNA, 22 nt, Artificial Sequence
  Synthetic Polynucleotide
agagaaggcauagaauaaagcc

SEQ ID NO: 380 - RNA, 22 nt, Artificial Sequence
  Synthetic Polynucleotide
aaaaugccagugagagaaggca

SEQ ID NO: 381 - RNA, 22 nt, Artificial Sequence
  Synthetic Polynucleotide
aagccaucggggcucucugcug

SEQ ID NO: 382 - RNA, 22 nt, Artificial Sequence
  Synthetic Polynucleotide
uccaucaucugugacucccuca

SEQ ID NO: 383 - RNA, 22 nt, Artificial Sequence
  Synthetic Polynucleotide
ucggggcucucugcugcugggu

SEQ ID NO: 384 - RNA, 22 nt, Artificial Sequence
  Synthetic Polynucleotide
uccaugccuggagucaggguug

SEQ ID NO: 385 - RNA, 22 nt, Artificial Sequence
  Synthetic Polynucleotide
ucccugagggagucacagauga

SEQ ID NO: 386 - RNA, 22 nt, Artificial Sequence
  Synthetic Polynucleotide
ucaucuucuccaugccuggagu

SEQ ID NO: 387 - RNA, 22 nt, Artificial Sequence
  Synthetic Polynucleotide
caguaucauaaccuccguccuu

SEQ ID NO: 388 - RNA, 22 nt, Artificial Sequence
  Synthetic Polynucleotide
gaaagucuucuuuuguccugca

SEQ ID NO: 389 - RNA, 22 nt, Artificial Sequence
  Synthetic Polynucleotide
caaaagaagacuuucuugucag

SEQ ID NO: 390 - RNA, 22 nt, Artificial Sequence
  Synthetic Polynucleotide
ccacacuauaaugcagaacucg

SEQ ID NO: 391 - RNA, 22 nt, Artificial Sequence
  Synthetic Polynucleotide
caugcuccagauaggcaacauc

SEQ ID NO: 392 - RNA, 22 nt, Artificial Sequence
  Synthetic Polynucleotide
agggauccgucacaagcccaaa

SEQ ID NO: 393 - RNA, 22 nt, Artificial Sequence
  Synthetic Polynucleotide
ugaucugggauugcugccccau

SEQ ID NO: 394 - RNA, 22 nt, Artificial Sequence
  Synthetic Polynucleotide
cuugucucagaaguaucugauc

SEQ ID NO: 395 - RNA, 22 nt, Artificial Sequence
  Synthetic Polynucleotide
gacaauucucuuugggcuugug

SEQ ID NO: 396 - RNA, 22 nt, Artificial Sequence
  Synthetic Polynucleotide
cuucugagacaagcuggucaag

SEQ ID NO: 397 - RNA, 22 nt, Artificial Sequence
  Synthetic Polynucleotide
aaggugauggugagcucugcaa

SEQ ID NO: 398 - RNA, 22 nt, Artificial Sequence
  Synthetic Polynucleotide
ugggcacuucuguucagacucc

SEQ ID NO: 399 - RNA, 22 nt, Artificial Sequence
  Synthetic Polynucleotide
guaaaaaauaugguaaagaccu

SEQ ID NO: 400 - RNA, 22 nt, Artificial Sequence
  Synthetic Polynucleotide
auacuauuauuuccagaagaag

SEQ ID NO: 401 - DNA, 57 bp, Artificial Sequence
  Synthetic Polynucleotide
tcgtcggcagcgtcagatgtgtataagagacagcatccaggcctcttatgtgaggag

SEQ ID NO: 402 - DNA, 57 bp, Artificial Sequence
  Synthetic Polynucleotide
gtctcgtgggctcggagatgtgtataagagacagatagatgaagggcagctgtttgc

SEQ ID NO: 403 - DNA, 55 bp, Artificial Sequence
  Synthetic Polynucleotide
tcgtcggcagcgtcagatgtgtataagagacagtgaacaacgcattgctgaaaga

SEQ ID NO: 404 - DNA, 58 bp, Artificial Sequence
  Synthetic Polynucleotide
gtctcgtgggctcggagatgtgtataagagacagttttatacagaaggaagccaacag

SEQ ID NO: 405 - DNA, 57 bp, Artificial Sequence
  Synthetic Polynucleotide
tcgtcggcagcgtcagatgtgtataagagacagaactgctgatattgatgtgaaaaa

SEQ ID NO: 406 - DNA, 61 bp, Artificial Sequence
  Synthetic Polynucleotide
gtctcgtgggctcggagatgtgtataagagacagaaaatagcaaaaattacaccataaagt

SEQ ID NO: 407 - DNA, 60 bp, Artificial Sequence
  Synthetic Polynucleotide
tcgtcggcagcgtcagatgtgtataagagacagtcctcaaatatttcaaattcccactgt

SEQ ID NO: 408 - DNA, 59 bp, Artificial Sequence
  Synthetic Polynucleotide
gtctcgtgggctcggagatgtgtataagagacagtccccatcttcataaatgcagtaac

SEQ ID NO: 409 - DNA, 60 bp, Artificial Sequence
  Synthetic Polynucleotide
tcgtcggcagcgtcagatgtgtataagagacagaaagatttacatggtggttgtattctt

SEQ ID NO: 410 - DNA, 56 bp, Artificial Sequence
  Synthetic Polynucleotide
gtctcgtgggctcggagatgtgtataagagacagctgtgctgcctgagattttcat

SEQ ID NO: 411 - DNA, 56 bp, Artificial Sequence
  Synthetic Polynucleotide
tcgtcggcagcgtcagatgtgtataagagacagagccccaaacgtagaaaatacct

SEQ ID NO: 412 - DNA, 59 bp, Artificial Sequence
  Synthetic Polynucleotide
gtctcgtgggctcggagatgtgtataagagacagctcccaaatagttggagttatgagt

SEQ ID NO: 413 - DNA, 57 bp, Artificial Sequence
  Synthetic Polynucleotide
tcgtcggcagcgtcagatgtgtataagagacaggtttcgattcagaggctttatgtc

SEQ ID NO: 414 - DNA, 61 bp, Artificial Sequence
  Synthetic Polynucleotide
gtctcgtgggctcggagatgtgtataagagacagctctctagggtattcattatgctttct

SEQ ID NO: 415 - DNA, 56 bp, Artificial Sequence
  Synthetic Polynucleotide
tcgtcggcagcgtcagatgtgtataagagacaggaagctttctgatgtcatgatcc

SEQ ID NO: 416 - DNA, 61 bp, Artificial Sequence
  Synthetic Polynucleotide
gtctcgtgggctcggagatgtgtataagagacaggcaacatttcattattaaaagagagca

SEQ ID NO: 417 - DNA, 54 bp, Artificial Sequence
  Synthetic Polynucleotide
tcgtcggcagcgtcagatgtgtataagagacagggaccaggcctgaatttgtag

SEQ ID NO: 418 - DNA, 57 bp, Artificial Sequence
  Synthetic Polynucleotide
gtctcgtgggctcggagatgtgtataagagacagtttgcaaactgactgaacattct

SEQ ID NO: 419 - DNA, 54 bp, Artificial Sequence
  Synthetic Polynucleotide
tcgtcggcagcgtcagatgtgtataagagacaggtcctgaagacactctcacct

SEQ ID NO: 420 - DNA, 56 bp, Artificial Sequence
  Synthetic Polynucleotide
gtctcgtgggctcggagatgtgtataagagacagcaggctcttaacatacaccagg

SEQ ID NO: 421 - DNA, 58 bp, Artificial Sequence
  Synthetic Polynucleotide
tcgtcggcagcgtcagatgtgtataagagacagtgctcatgcctgtcaaattgaaata

SEQ ID NO: 422 - DNA, 63 bp, Artificial Sequence
  Synthetic Polynucleotide
gtctcgtgggctcggagatgtgtataagagacagacatctgttgaaattctaattctttctgt

SEQ ID NO: 423 - DNA, 52 bp, Artificial Sequence
  Synthetic Polynucleotide
tcgtcggcagcgtcagatgtgtataagagacagctagacgctgcgtgctgct

SEQ ID NO: 424 - DNA, 56 bp, Artificial Sequence
  Synthetic Polynucleotide
gtctcgtgggctcggagatgtgtataagagacagaaggccaagcatataccgcaga

SEQ ID NO: 425 - DNA, 55 bp, Artificial Sequence
  Synthetic Polynucleotide
tcgtcggcagcgtcagatgtgtataagagacagatgtcctgtcctagggtttcct

SEQ ID NO: 426 - DNA, 56 bp, Artificial Sequence
  Synthetic Polynucleotide
gtctcgtgggctcggagatgtgtataagagacagtcagcatctcccttttcctctc

SEQ ID NO: 427 - DNA, 55 bp, Artificial Sequence
  Synthetic Polynucleotide
tcgtcggcagcgtcagatgtgtataagagacagggcagcggctgaatatacaagt

SEQ ID NO: 428 - DNA, 53 bp, Artificial Sequence
  Synthetic Polynucleotide
gtctcgtgggctcggagatgtgtataagagacagctcgcctatgcccttcgac

SEQ ID NO: 429 - DNA, 57 bp, Artificial Sequence
  Synthetic Polynucleotide
tcgtcggcagcgtcagatgtgtataagagacagagaatcaaaagaagctctccagtg

SEQ ID NO: 430 - DNA, 55 bp, Artificial Sequence
  Synthetic Polynucleotide
gtctcgtgggctcggagatgtgtataagagacagtcactcactatcctctcccga

SEQ ID NO: 431 - DNA, 58 bp, Artificial Sequence
  Synthetic Polynucleotide
tcgtcggcagcgtcagatgtgtataagagacagagtgtacttctatcagtaggtgctt

SEQ ID NO: 432 - DNA, 54 bp, Artificial Sequence
  Synthetic Polynucleotide
gtctcgtgggctcggagatgtgtataagagacaggacctactggcttggctgat

SEQ ID NO: 433 - DNA, 59 bp, Artificial Sequence
  Synthetic Polynucleotide
tcgtcggcagcgtcagatgtgtataagagacagaagcagcagaacaagtctttttagtt

SEQ ID NO: 434 - DNA, 53 bp, Artificial Sequence
  Synthetic Polynucleotide
gtctcgtgggctcggagatgtgtataagagacagaccagggagaccacaccgt

SEQ ID NO: 435 - DNA, 57 bp, Artificial Sequence
  Synthetic Polynucleotide
tcgtcggcagcgtcagatgtgtataagagacagacagcatttttggagacaatgaga

SEQ ID NO: 436 - DNA, 57 bp, Artificial Sequence
  Synthetic Polynucleotide
gtctcgtgggctcggagatgtgtataagagacagatgcctgagctatgtaaaacgtc

SEQ ID NO: 437 - DNA, 54 bp, Artificial Sequence
  Synthetic Polynucleotide
tcgtcggcagcgtcagatgtgtataagagacagcccagcaatctaggctctact

SEQ ID NO: 438 - DNA, 59 bp, Artificial Sequence
  Synthetic Polynucleotide
gtctcgtgggctcggagatgtgtataagagacagccttccacagtgtttgttaatatgc

SEQ ID NO: 439 - DNA, 57 bp, Artificial Sequence
  Synthetic Polynucleotide
tcgtcggcagcgtcagatgtgtataagagacagtcaaatacacaagaaaaggcgttg

SEQ ID NO: 440 - DNA, 58 bp, Artificial Sequence
  Synthetic Polynucleotide
gtctcgtgggctcggagatgtgtataagagacagacaattccatcagtatccattggt

SEQ ID NO: 441 - DNA, 58 bp, Artificial Sequence
  Synthetic Polynucleotide
tcgtcggcagcgtcagatgtgtataagagacagggttaggagtgaaacagacaaatgg

SEQ ID NO: 442 - DNA, 61 bp, Artificial Sequence
  Synthetic Polynucleotide
gtctcgtgggctcggagatgtgtataagagacagtggtgttccatagccataaataatgtg

SEQ ID NO: 443 - DNA, 57 bp, Artificial Sequence
  Synthetic Polynucleotide
tcgtcggcagcgtcagatgtgtataagagacagtcttgatctggaattgctctccat

SEQ ID NO: 444 - DNA, 62 bp, Artificial Sequence
  Synthetic Polynucleotide
gtctcgtgggctcggagatgtgtataagagacagacaatgatgacaactaaaaagagaaact

SEQ ID NO: 445 - DNA, 59 bp, Artificial Sequence
  Synthetic Polynucleotide
tcgtcggcagcgtcagatgtgtataagagacagatcattgtgttgatttcctgttttct

SEQ ID NO: 446 - DNA, 56 bp, Artificial Sequence
  Synthetic Polynucleotide
gtctcgtgggctcggagatgtgtataagagacagccagtctgaatgatcgcagaac

SEQ ID NO: 447 - DNA, 59 bp, Artificial Sequence
  Synthetic Polynucleotide
tcgtcggcagcgtcagatgtgtataagagacagtgcagctgaaatggtattaaaactga

SEQ ID NO: 448 - DNA, 58 bp, Artificial Sequence
  Synthetic Polynucleotide
gtctcgtgggctcggagatgtgtataagagacagtgcaaaaccaaagaaatacccctt

SEQ ID NO: 449 - DNA, 53 bp, Artificial Sequence
  Synthetic Polynucleotide
tcgtcggcagcgtcagatgtgtataagagacaggctgtcacctctctgtggtt

SEQ ID NO: 450 - DNA, 54 bp, Artificial Sequence
  Synthetic Polynucleotide
gtctcgtgggctcggagatgtgtataagagacagtagaacttgggcagctggtt

SEQ ID NO: 451 - DNA, 53 bp, Artificial Sequence
  Synthetic Polynucleotide
tcgtcggcagcgtcagatgtgtataagagacagccaagcagggaacaaagaaa

SEQ ID NO: 452 - DNA, 54 bp, Artificial Sequence
  Synthetic Polynucleotide
gtctcgtgggctcggagatgtgtataagagacagccgagacttcctctccaaga

SEQ ID NO: 453 - DNA, 56 bp, Artificial Sequence
  Synthetic Polynucleotide
tcgtcggcagcgtcagatgtgtataagagacaggcctgagataatgcctctcatgt

SEQ ID NO: 454 - DNA, 54 bp, Artificial Sequence
  Synthetic Polynucleotide
gtctcgtgggctcggagatgtgtataagagacagactggacacagtaggcaagg

SEQ ID NO: 455 - DNA, 56 bp, Artificial Sequence
  Synthetic Polynucleotide
tcgtcggcagcgtcagatgtgtataagagacagtgtaatcattcagcatcaaggtg

SEQ ID NO: 456 - DNA, 56 bp, Artificial Sequence
  Synthetic Polynucleotide
gtctcgtgggctcggagatgtgtataagagacagacaaagactgccaagtgaagga

SEQ ID NO: 457 - DNA, 53 bp, Artificial Sequence
  Synthetic Polynucleotide
tcgtcggcagcgtcagatgtgtataagagacagcatccctccctcctggaaga

SEQ ID NO: 458 - DNA, 55 bp, Artificial Sequence
  Synthetic Polynucleotide
gtctcgtgggctcggagatgtgtataagagacagcccctctcctatcacacatgc

SEQ ID NO: 459 - DNA, 53 bp, Artificial Sequence
  Synthetic Polynucleotide
tcgtcggcagcgtcagatgtgtataagagacagaggctaatgataccccaggt

SEQ ID NO: 460 - DNA, 54 bp, Artificial Sequence
  Synthetic Polynucleotide
gtctcgtgggctcggagatgtgtataagagacagagtctttgccctggaacctt

SEQ ID NO: 461 - DNA, 53 bp, Artificial Sequence
  Synthetic Polynucleotide
tcgtcggcagcgtcagatgtgtataagagacaggtgtgctccgtgtgtgctac

SEQ ID NO: 462 - DNA, 55 bp, Artificial Sequence
  Synthetic Polynucleotide
gtctcgtgggctcggagatgtgtataagagacagaaccagaccttggtccctatg

SEQ ID NO: 463 - DNA, 53 bp, Artificial Sequence
  Synthetic Polynucleotide
tcgtcggcagcgtcagatgtgtataagagacagcttcaagggcaacctcaaaa

SEQ ID NO: 464 - DNA, 54 bp, Artificial Sequence
  Synthetic Polynucleotide
gtctcgtgggctcggagatgtgtataagagacagatttccttgcaagagggatg

SEQ ID NO: 465 - DNA, 53 bp, Artificial Sequence
  Synthetic Polynucleotide
tcgtcggcagcgtcagatgtgtataagagacagattgcattcaccacacaagg

SEQ ID NO: 466 - DNA, 54 bp, Artificial Sequence
  Synthetic Polynucleotide
gtctcgtgggctcggagatgtgtataagagacagatgccaaggacaagatggag

SEQ ID NO: 467 - DNA, 53 bp, Artificial Sequence
  Synthetic Polynucleotide
tcgtcggcagcgtcagatgtgtataagagacagtgggctaccttgtctgcaat

SEQ ID NO: 468 - DNA, 53 bp, Artificial Sequence
  Synthetic Polynucleotide
gtctcgtgggctcggagatgtgtataagagacagagcctccaaccaagtctgc

SEQ ID NO: 469 - DNA, 54 bp, Artificial Sequence
  Synthetic Polynucleotide
tcgtcggcagcgtcagatgtgtataagagacagcctggaggaggctgacttaaa

SEQ ID NO: 470 - DNA, 54 bp, Artificial Sequence
  Synthetic Polynucleotide
gtctcgtgggctcggagatgtgtataagagacagagggcctcctggaacttctt

SEQ ID NO: 471 - DNA, 53 bp, Artificial Sequence
  Synthetic Polynucleotide
tcgtcggcagcgtcagatgtgtataagagacagtgcagaccctgaggacttct

SEQ ID NO: 472 - DNA, 54 bp, Artificial Sequence
  Synthetic Polynucleotide
gtctcgtgggctcggagatgtgtataagagacagtggacagtctgcaaccttct

SEQ ID NO: 473 - DNA, 57 bp, Artificial Sequence
  Synthetic Polynucleotide
tcgtcggcagcgtcagatgtgtataagagacagtcatgctaagtccaagcaaatact

SEQ ID NO: 474 - DNA, 54 bp, Artificial Sequence
  Synthetic Polynucleotide
gtctcgtgggctcggagatgtgtataagagacaggcagctgcaatggtgggtaa

SEQ ID NO: 475 - DNA, 54 bp, Artificial Sequence
  Synthetic Polynucleotide
tcgtcggcagcgtcagatgtgtataagagacagttccagccttcttgctccttt

SEQ ID NO: 476 - DNA, 54 bp, Artificial Sequence
  Synthetic Polynucleotide
gtctcgtgggctcggagatgtgtataagagacagagtcagggttgctgggttga

SEQ ID NO: 477 - DNA, 53 bp, Artificial Sequence
  Synthetic Polynucleotide
tcgtcggcagcgtcagatgtgtataagagacagccctgagggagtcacagatg

SEQ ID NO: 478 - DNA, 54 bp, Artificial Sequence
  Synthetic Polynucleotide
gtctcgtgggctcggagatgtgtataagagacaggctcaaggcttctaggtgga

SEQ ID NO: 479 - DNA, 53 bp, Artificial Sequence
  Synthetic Polynucleotide
tcgtcggcagcgtcagatgtgtataagagacagtttgccatgaagatgtcagg

SEQ ID NO: 480 - DNA, 54 bp, Artificial Sequence
  Synthetic Polynucleotide
gtctcgtgggctcggagatgtgtataagagacagtgctcacatgggaattcatc

SEQ ID NO: 481 - DNA, 53 bp, Artificial Sequence
  Synthetic Polynucleotide
tcgtcggcagcgtcagatgtgtataagagacagagaggatgaccgcagaattg

SEQ ID NO: 482 - DNA, 54 bp, Artificial Sequence
  Synthetic Polynucleotide
gtctcgtgggctcggagatgtgtataagagacagatgcacaaggtgatggtgag

SEQ ID NO: 483 - DNA, 55 bp, Artificial Sequence
  Synthetic Polynucleotide
tcgtcggcagcgtcagatgtgtataagagacagcttgaccagcttgtctcagaag

SEQ ID NO: 484 - DNA, 52 bp, Artificial Sequence
  Synthetic Polynucleotide
gtctcgtgggctcggagatgtgtataagagacagactgcaccctgccatcat

SEQ ID NO: 485 - DNA, 52 bp, Artificial Sequence
  Synthetic Polynucleotide
tcgtcggcagcgtcagatgtgtataagagacagaccccacagatcccactgt

SEQ ID NO: 486 - DNA, 55 bp, Artificial Sequence
  Synthetic Polynucleotide
gtctcgtgggctcggagatgtgtataagagacagcacagacaggcttcccttctt

SEQ ID NO: 487 - DNA, 55 bp, Artificial Sequence
  Synthetic Polynucleotide
tcgtcggcagcgtcagatgtgtataagagacagtgctgaaatggtcttcaaaatc

SEQ ID NO: 488 - DNA, 54 bp, Artificial Sequence
  Synthetic Polynucleotide
gtctcgtgggctcggagatgtgtataagagacagtgaatctgggtgggagtttc

SEQ ID NO: 489 - DNA, 23 bp, Artificial Sequence
  Synthetic Polynucleotide
  misc_feature (1)..(21): n is a, c, g, or t
  misc_feature (22)..(22): r is a or g
nnnnnnnnnnnnnnnnnnnnnrg

SEQ ID NO: 490 - DNA, 28 bp, Artificial Sequence
  Synthetic Polynucleotide
  misc_feature (1)..(24): n is a, c, g, or t
  misc_feature (26)..(27): r is a or g
nnnnnnnnnnnnnnnnnnnnnnnngrrt

SEQ ID NO: 491 - RNA, 20 nt, Artificial Sequence
  Synthetic Polynucleotide
uucccguucaccggcagcau

SEQ ID NO: 492 - RNA, 20 nt, Artificial Sequence
  Synthetic Polynucleotide
guucaccggcagcauuggug

SEQ ID NO: 493 - RNA, 20 nt, Artificial Sequence
  Synthetic Polynucleotide
uugguggggaccuacuggcu

SEQ ID NO: 494 - RNA, 20 nt, Artificial Sequence
  Synthetic Polynucleotide
uagguccccaccaaugcugc

SEQ ID NO: 495 - RNA, 20 nt, Artificial Sequence
  Synthetic Polynucleotide
gcuguccauuggggagcaug

SEQ ID NO: 496 - RNA, 20 nt, Artificial Sequence
  Synthetic Polynucleotide
ucuggcagaagcuguccauu

SEQ ID NO: 497 - RNA, 20 nt, Artificial Sequence
  Synthetic Polynucleotide
gcaagcucacuagugggcgg

SEQ ID NO: 498 - DNA, 20 bp, Artificial Sequence
  Synthetic Polynucleotide
agagcaacagtgctgtggcc

SEQ ID NO: 499 - RNA, 20 nt, Artificial Sequence
  Synthetic Polynucleotide
agagcaacagugcuguggcc